(19) United States
(12) Patent Application Publication
(10) Pub. No.: US 2024/0391900 A1
(43) Pub. Date: Nov. 28, 2024

(54) RIP1 KINASE INHIBITOR AND USE THEREOF

(71) Applicant: SIRONAX (BEIJING) CO., LTD., Beijing (CN)

(72) Inventors: Yaning Su, Beijing (CN); Hanying Ruan, Beijing (CN); Zhiyuan Zhang, Beijing (CN); Yanping Xu, Beijing (CN); Yimin Jianng, Beijing (CN)

(21) Appl. No.: 18/692,053

(22) PCT Filed: Sep. 14, 2022
(86) PCT No.: PCT/CN2022/118631

§ 371 Date: Mar. 14, 2024

(30) Foreign Application Priority Data
Sep. 16, 2021 (WO) PCT/CN2021/118770

(51) Int. Cl. C07D 401/14; A61K 31/416; A61K 31/4178; A61K 31/437; A61K 31/4439; A61K 31/444; A61K 31/497; A61K 31/501; A61K 31/5025; A61K 31/506; C07D 401/04; C07D 401/10; C07D 403/08; C07D 403/10; C07D 417/14; C07D 471/04; C07D 487/04; C07D 491/048

(52) U.S. Cl. CPC C07D 401/14; A61K 31/416; A61K 31/4178; A61K 31/437; A61K 31/4439; A61K 31/444; A61K 31/497; A61K 31/501; A61K 31/5025; A61K 31/506; C07D 401/04; C07D 401/10; C07D 403/08; C07D 403/10; C07D 417/14; C07D 471/04; C07D 487/04; C07D 491/048

(57) ABSTRACT

Provided are novel compounds of general formula (I) that inhibit RIP1 kinase or a pharmaceutically acceptable salt thereof, a composition comprising such compounds, and use of such compounds in inhibiting programmed necrosis, inhibiting RIP1 kinase, or treating or preventing a disease that is at least partially mediated by RIP1 kinase.

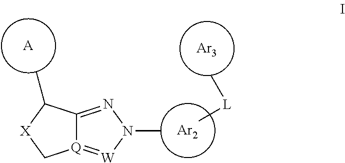

### TECHNICAL FIELD

The present disclosure relates to a novel compound that inhibits RIP1 kinase or a pharmaceutically acceptable salt thereof, a composition comprising such a compound, and use of such a compound in inhibiting programmed necrosis, inhibiting RIP1 kinase, or treating or preventing a disease that is at least partially mediated by RIP1 kinase.

### BACKGROUND

Necrosis has long been considered a passive and uncontrollable process. However, as people continue to study the death mechanism, in some cases, there is a regulated and orderly death mode with necrosis-like morphological changes. In 2005, Degterev et al. discovered a small molecule substance, Necrostatin-1 (Nec-1), which can specifically inhibit the cell death triggered by the death receptor signaling and induced independently on an aspartate-specific cysteine protease (caspase). This cell death mode that can be specifically inhibited by Nec-1 is named programmed necrosis, also known as necroptosis (Nat Chem Biol 2005; 15 1:112-119). Programmed necrosis is a new modulated cell death mode with necrotizing morphological characteristics. Programmed necrosis plays a key role in embryonic development and homeostasis in the adult organism, as well as in various pathological forms of cell death of multiple diseases such as ischemic brain injury, neurodegenerative diseases, and viral infections (Am. J. Pathol. 2020. 190, 2, 272-285).

The receptor-interacting protein 1 (RIP1) family is a class of serine/threonine protein kinases with a relatively conserved kinase domain but distinct non-kinase regions. The RIP family includes 7 members, namely RIP1-RIP7, of which RIP1 is the most reported and most widely studied member. RIP1 contains a C-terminal death domain, an N-terminal serine/threonine kinase domain, and an intermediate domain that mediates the activation of nuclear factor κB (NF-κB). NF-κB activation induced by tumor necrosis factor α (TNF-α) plays a central role in the immune system and inflammatory response. The kinase activity of RIP1 is critically involved in mediating the programmed necrosis of cells, a caspase-independent programmed cell death pathway.

RIP1 is a multifunctional signal transducer involved in mediating nuclear factor κB (NF-κB) activation, apoptosis and necrosis, and is located at a key position in the programmed necrosis pathway. As a regulatory molecule upstream of the signaling pathway, abnormal activation of RIP1 can cause a series of responses, so it has become the “central controller” for determining the cell fate in the death receptor signaling pathway. After TNF-α stimulates tumor necrosis factor α receptor 1 (TNF-α receptor 1, TNFR1), the trimerization of TNFR1 is triggered, conformational changes occur, which in turn cause the intracellular domain of TNFR1 to recruit a variety of proteins to form Complex I, this molecular complex including TNFR-receptor-associated death domain (TRADD), RIP1, cellular inhibitor of apoptosis proteins 1 (cIAPI), cIAP2, TNFR-associated factor 2 (TRAF2) and TRAF5. In Complex I, RIP1 is rapidly modified by the ubiquitination in multiple forms, thereby activating the NF-κB pathway, in which the ubiquitinated RIP1 functions as an essential regulatory protein of NF-κB. Without survival signal transduction, the membrane-associated Complex I transforms to the cytoplasmic Complex IIa, which contains Fas-associated death domain protein (FADD), RIP1, and caspase-8, thereby activating the apoptosis pathway; if the apoptosis is blocked by a caspase inhibitor, the complex IIb including RIP1, RIP3 and human mixed lineage kinase domain-like protein (MLKL) will be formed in the cells, and the death signal was transmitted to downstream, allowing the programmed necrosis to eventually occur. Among others, the kinase activity of RIP1 is necessary for the formation of the Complex IIb. Programmed necrosis of cells releases contents to the surroundings. As damage-associated molecular patterns (DAMPs), these contents can stimulate inflammatory responses in surrounding cells and activate the collective immune responses.

RIP1 kinase is a potential target in the programmed necrosis pathway, and inhibiting its kinase activity can inhibit the progression of the disease. Therefore, RIP1 kinase is recognized as a potential therapeutic target for diseases relating to programmed necrosis of cells. Necrostatin-1 (Nec-1), the first small molecule inhibitor of RIP1 kinase activity, can prevent necroptosis (Nat Chem Biol 2005; 15 1:112-119). Preclinical studies have found that Nec-1 and its analogs show therapeutic effects in a variety of neurodegenerative diseases, inflammation, cancer and other diseases. For example, Nec-1 and its analogs have a relieving effect on Alzheimer's disease (AD), amyotrophic lateral sclerosis (ALS), multiple sclerosis (MS), Parkinson's disease (PD), and the like; can alleviate ischemic brain injury, ischemic myocardial injury, retinal ischemia/reperfusion injury, glaucoma, renal ischemia-reperfusion injury; and have protective effects against psoriasis, retinitis pigmentosa, inflammatory bowel disease, autoimmune diseases, caerulin-induced acute pancreatitis and sepsis or systemic inflammatory response syndrome (SIRS). Therefore, development on drugs targeting RIP1 has become a hot topic in current new drug research.

With the in-depth research on programmed necrosis and RIP1, RIP1 inhibitors have received great attention from medicinal chemistry researchers, and many RIP1 inhibitors have been reported successively. Berger et al. of GlaxoSmithKline (GSK) obtained the small molecule inhibitor GSK′ 963 through high-throughput screening, which can effectively block programmed necrosis of mouse L929 cells and human U937 cells, with IC50 values of 1 and 4 nmol-IL respectively. In the TNF-induced shock model, GSK′963 can effectively inhibit hypothermia and avoid the effects of hypothermia on mice. For rodents, in spite of higher activity and selectivity, exposure to these inhibitors is extremely low from oral administration. GSK2982772, which was later developed by GSK, has excellent activity data and pharmacokinetic properties and has completed Phase II clinical trials. Its indications are ulcerative colitis, rheumatoid arthritis and plaque psoriasis. However, GSK2982772 has the drawback of low brain tissue distribution. So far, multiple new RIP1 kinase inhibitors have been reported successively (J. Med. Chem. 2020, 63, 4, 1490-1510). For example, biopharmaceutical companies such as Genentech, Denali and Rigel have successively discovered multiple RIP1 inhibitors such as DNL-747, DNL-758 and R552. Although the research on RIP1 kinase inhibitors has made important progress, from the experimental data published so far, the inventors believe that there are still many problems to be solved in this field, such as poor selectivity, poor pharmacokinetic properties, unstable metabolism, lower oral bioavailability, few RIP kinase inhibitors with high selectivity and activity, and some compounds failing to penetrate the blood-brain barrier and enter the central nervous system. These shortcomings limit its further research and clinical application.

An effective small molecule RIP1 kinase activity inhibitor should be able to block RIP1-dependent programmed necrosis of cells, thereby allowing to provide therapeutic effects for diseases or events related to necrosis or inflammatory response. Therefore, the development of small molecule RIP1 kinase inhibitors with clinical application value that are highly specific, highly active, and have blood-brain penetration is currently a hot and difficult point in the treatment of diseases relating to programmed necrosis of cells.

### SUMMARY OF THE INVENTION

### Brief Description

The present disclosure provides structurally novel small molecule RIP1 kinase inhibitors (hereinafter referred to as compounds of the present disclosure).

Accordingly, in a first aspect of the present disclosure there is provided a compound of general formula (I) or a pharmaceutically acceptable salt, hydrate, solvate and stereoisomer thereof:

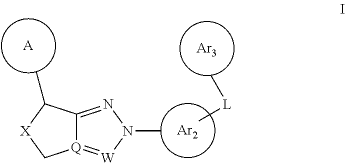

wherein,

Ring A is C₆-C₁₂aryl; C₃-C₈cycloalkyl; or 5 to 12 membered heteroaryl containing 1 or 2 heteroatoms independently selected from N, O and S, wherein said aryl, cycloalkyl or heteroaryl is substituted with 0, 1, 2 or 3 substituents independently selected from halogen, C₁-C₆alkyl, haloC₁-C₆alkyl, C₂-C₆alkenyl, C₂-C₆alkynyl, C₁-C₆alkoxy, haloC₁-C₆alkoxy, cyano, hydroxy, nitro, —NR¹R², —C(═O)R¹, —C(═O)OR¹, —OC(═O)R¹, —C(═O)NR¹R², —SR¹, —S(═O)R¹, —S(═O)₂R¹ and —S(═O)₂NR¹R²;

Ring Ar₂ is C₆-C₁₂arylene; C₃-C₈cycloalkylene; 3 to 12 membered heterocyclylene containing 1 or 2 heteroatoms independently selected from N, O and S; or 5 to 12 membered heteroarylene containing 1 or 2 heteroatoms independently selected from N, O and S, wherein said arylene, cycloalkylene, heterocyclylene or heteroarylene is substituted with 0, 1, 2 or 3 substituents independently selected from halogen, C₁-C₆alkyl, haloC₁-C₆alkyl, C₂-C₆alkenyl, C₂-C₆alkynyl, C₁-C₆alkoxy, haloC₁-C₆alkoxy, cyano, hydroxy, nitro, —NR¹R², —C(═O)R¹, —C(═O)OR¹, —OC(═O)R¹, —C(═O)NR¹R², —SR¹, —S(═O)R¹, —S(═O)₂R¹ and —S(═O)₂NR¹R², and wherein said heterocyclylene is optionally further substituted with one ═O group;

Ring Ar₃ is C₆-C₁₂aryl; 3 to 12 membered heterocyclyl containing 1 or 2 heteroatoms independently selected from N, O and S; or 5 to 12 membered heteroaryl containing 1 or 2 heteroatoms independently selected from N, O and S, wherein said aryl, heterocyclyl or heteroaryl is substituted with 0, 1, 2 or 3 substituents independently selected from halogen, C₁-C₆alkyl, haloC₁-C₆alkyl, C₂-C₆alkenyl, C₂-C₆alkynyl, C₁-C₆alkoxy, haloC₁-C₆alkoxy, cyano, hydroxy, nitro, —NR¹R², —C(═O)R¹, —C(═O)OR¹, —OC(═O)R¹, —C(═O)NR¹R², —SR¹, —S(═O)R¹, —S(═O)₂R¹ and —S(═O)₂NR¹R², and wherein said heterocyclyl is optionally further substituted with one ═O group;

L is selected from C₁-C₆alkylene, C₂-C₆alkenylene or C₂-C₆alkynylene;

X is CR¹R², O or S;

Q is C or N;

W is CR³, —C(═O)— or N;

the dashed line between Q and W represents a single bond or a double bond, provided that when Q is C, the dashed line between Q and W represents a double bond and at the same time W is CR³ or N, and when Q is N, the dashed line between Q and W represents a single bond and at the same time W is —C(═O)—;

each of R¹ and R² is independently H or C₁-C₆alkyl; and

R³ is H, halogen, C₁-C₆alkyl, haloC₁-C₆alkyl, C₁-C₆alkoxy, haloC₁-C₆alkoxy, cyano, hydroxy, nitro or —NR¹R².

In a second aspect of the present disclosure there is provided a pharmaceutical composition, comprising a compound of general formula (I), or a pharmaceutically acceptable salt, hydrate, solvate and stereoisomer thereof and a pharmaceutically acceptable excipient. In some embodiments according to this aspect, the pharmaceutical composition contains two or more pharmaceutically acceptable excipients. In some embodiments according to this aspect, the pharmaceutical composition is in the form of a pharmaceutically acceptable dosage form.

In a third aspect of the present disclosure there is provided a compound of general formula (I), or a pharmaceutically acceptable salt, hydrate, solvate and stereoisomer thereof, or a pharmaceutical composition comprising a compound of general formula (I), or a pharmaceutically acceptable salt, hydrate, solvate and stereoisomer thereof and a pharmaceutically acceptable excipient, for use in treating or preventing a disease.

In a fourth aspect of the present disclosure there is provided a compound of general formula (I), or a pharmaceutically acceptable salt, hydrate, solvate and stereoisomer thereof, or a pharmaceutical composition comprising a compound of general formula (I), or a pharmaceutically acceptable salt, hydrate, solvate and stereoisomer thereof and a pharmaceutically acceptable excipient, for use in inhibiting programmed necrosis, inhibiting RIP1 kinase, or treating or preventing a disease that is at least partially mediated by RIP1 kinase.

In a fifth aspect of the present disclosure there is provided use of a compound of general formula (I), or a pharmaceutically acceptable salt, hydrate, solvate and stereoisomer thereof, or a pharmaceutical composition comprising a compound of general formula (I), or a pharmaceutically acceptable salt, hydrate, solvate and stereoisomer thereof and a pharmaceutically acceptable excipient for inhibiting programmed necrosis, inhibiting RIP1 kinase, or treating or preventing a disease that is at least partially mediated by RIP1 kinase.

In a sixth aspect of the present disclosure there is provided use of a compound of general formula (I), or a pharmaceutically acceptable salt, hydrate, solvate and stereoisomer thereof, or a pharmaceutical composition comprising a compound of general formula (I), or a pharmaceutically acceptable salt, hydrate, solvate and stereoisomer thereof and a pharmaceutically acceptable excipient in manufacture of a medicament for inhibiting programmed necrosis, inhibiting RIP1 kinase, or treating or preventing a disease that is at least partially mediated by RIP1 kinase.

In a seventh aspect of the present disclosure there is provided a method for inhibiting programmed necrosis, inhibiting RIP1 kinase, or treating or preventing a disease that is at least partially mediated by RIP1 kinase, which method comprises administering to a subject in need thereof an effective amount of a compound of general formula (I), or a pharmaceutically acceptable salt, hydrate, solvate and stereoisomer thereof, or a pharmaceutical composition comprising a compound of general formula (I), or a pharmaceutically acceptable salt, hydrate, solvate and stereoisomer thereof and a pharmaceutically acceptable excipient.

### Definition

It is to be understood that unless otherwise expressly defined herein, terms used herein shall be given their generally accepted meanings known in the relevant art. It is further to be understood that the terminology used herein is intended to describe particular embodiments only and is not intended to be limiting.

Unless stated otherwise, the singular forms “a”, “an” and “the” as used herein include the plural forms.

Unless stated otherwise, the term “comprises” or “consisting essentially of” includes the case of “consisting of”.

The prefix “Cₓ-C_{y}” to a group term refers to the range of carbon atoms contained in the group (the endpoints of the range and each integer contained in the range and any subranges formed by these integers are intended to be included within the scope of this disclosure). For example, “C₁-C₆ alkyl” means an alkyl group containing 1 to 6 carbon atoms.

The term “compounds of the present disclosure” as used herein refers to any one or more compounds falling within the scope of compound of general formula (I) or a pharmaceutically acceptable salt, hydrate, solvate and stereoisomer thereof.

The term “alkyl” as used herein refers to a straight or branched, saturated monovalent hydrocarbon group having a specified number of carbon atoms. An alkyl group typically contains 1-12 carbon atoms (“C₁-C₁₂alkyl”), for example 1-8 carbon atoms (“C₁-C₈alkyl”), preferably 1-6 carbon atoms (“C₁-C₆alkyl”), more preferably 1-5 carbon atoms (“C₁-C₅alkyl”), 1-4 carbon atoms (“C₁-C₄alkyl”) or 1-2 carbon atoms (“C₁-C₂alkyl”). Examples of an alkyl group include methyl, ethyl, n-propyl, iso-propyl, n-butyl, sec-butyl, iso-butyl, tert-butyl, n-pentyl, iso-pentyl, neo-pentyl, n-hexyl, n-heptyl, n-octyl and the like. Preferred C₁-C₆alkyl groups include methyl, ethyl, n-propyl, iso-propyl, n-butyl, sec-butyl, iso-butyl, tert-butyl, n-pentyl, iso-pentyl, neo-pentyl, and n-hexyl. Preferred C₁-C₄alkyl groups include methyl, ethyl, n-propyl, iso-propyl, n-butyl, sec-butyl, iso-butyl, and tert-butyl.

The term “alkylene” as used herein refers to a straight or branched, saturated divalent hydrocarbon group having a specified number of carbon atoms. An alkylene group typically contains 1-12 carbon atoms (“C₁-C₁₂alkylene”), for example 1-8 carbon atoms (“C₁-C₈alkylene”), preferably 1-6 carbon atoms (“C₁-C₆alkylene”), more preferably 1-5 carbon atoms (“C₁-C₅alkylene”), 1-4 carbon atoms (“C₁-C₄alkylene”) or 1-2 carbon atoms (“C₁-C₂alkylene”). Examples of an alkylene group include methylene, ethylene, n-propylene, iso-propylene, n-butylene, sec-butylene, iso-butylene, tert-butylene, n-pentylene, iso-pentylene, neo-pentylene, n-hexylene, n-heptylene, n-octylene and the like. Preferred C₁-C₆alkylene groups include methylene, ethylene, n-propylene, iso-propylene, n-butylene, sec-butylene, iso-butylene, tert-butylene, n-pentylene, iso-pentylene, neo-pentylene and n-hexylene. Preferred C₁-C₄alkylene groups include methylene, ethylene, n-propylene, iso-propylene, n-butylene, sec-butylene, iso-butylene, and tert-butylene.

The term “alkenyl” as used herein refers to a straight or branched unsaturated monovalent hydrocarbon group having a specified number of carbon atoms and containing one or more double bonds. An alkenyl group typically contains 2-12 carbon atoms (“C₂-C₁₂alkenyl”), for example 2-8 carbon atoms (“C₂-C₈alkenyl”), preferably 2-6 carbon atoms (“C₂-C₆alkenyl”), more preferably 2-5 carbon atoms (“C₂-C₅alkenyl”), 2-4 carbon atoms (“C₂-C₄alkenyl”) or 2 carbon atoms (“ethenyl”). Preferred C₂-C₆alkenyl groups include ethenyl, 1-propenyl, 2-propenyl, 1-butenyl, 2-butenyl, 1,3-butadien-1-yl, 1-penten-3-yl, 2-penten-1-yl, 3-penten-1-yl, 3-penten-2-yl, 1,3-pentadien-1-yl, 1,4-pentadien-3-yl, 1-hexen-3-yl, and 1,4-hexadien-1-yl.

The term “alkenylene” as used herein refers to a straight or branched unsaturated bivalent hydrocarbon group having a specified number of carbon atoms and containing one or more double bonds. An alkenylene group typically contains 2-12 carbon atoms (“C₂-C₁₂alkenylene”), for example 2-8 carbon atoms (“C₂-C₈alkenylene”), preferably 2-6 carbon atoms (“C₂-C₆alkenylene”), more preferably 2-5 carbon atoms (“C₂-C₅alkenylene”), 2-4 carbon atoms (“C₂-C₄alkenylene”) or 2 carbon atoms (“ethenylene”).

The term “alkynyl” as used herein refers to a straight or branched unsaturated monovalent hydrocarbon group having a specified number of carbon atoms and containing one or more triple bonds. An alkynyl group typically contains 2-12 carbon atoms (“C₂-C₁₂alkynyl”), for example 2-8 carbon atoms (“C₂-C₈alkynyl”), preferably 2-6 carbon atoms (“C₂-C₆alkynyl”), more preferably 2-5 carbon atoms (“C₂-C₅alkynyl”), 2-4 carbon atoms (“C₂-C₄alkynyl”) or 2 carbon atoms (“ethynyl”). Preferred C₂-C₆alkynyl groups include ethynyl, propynyl, butynyl, pentynyl, and hexynyl.

The term “alkynylene” as used herein refers to a straight or branched, unsaturated bivalent hydrocarbon group having a specified number of carbon atoms and containing one or more triple bonds. An alkynylene group typically contains 2-12 carbon atoms (“C₂-C₁₂alkynylene”), for example 2-8 carbon atoms (“C₂-C₈alkynylene”), preferably 2-6 carbon atoms (“C₂-C₆alkynylene”), more preferably 2-5 carbon atoms (“C₂-C₅alkynylene”), 2-4 carbon atoms (“C₂-C₄alkynylene”) or 2 carbon atoms (“ethynylene”).

The term “alkoxy” as used herein refers to an alkyl attached to the parent molecule through an oxygen atom (i.e., “—O-alkyl”), where “alkyl” is as defined above. An alkoxy group typically contains 1-8 carbon atoms (“C₁-C₈ alkoxy”), preferably 1-6 carbon atoms (“C₁-C₆ alkoxy”), more preferably 1-4 carbon atoms (“C₁-C₄ alkoxy”). For example, C₁-C₄ alkoxy groups include methoxy, ethoxy, n-propoxy, isopropoxy, n-butoxy, tert-butoxy, and the like.

The term “halogen” as used herein refers to fluoro, chloro, bromo, iodo, and the like, preferably fluoro, chloro.

The term “halogenated/halo” as used herein means that one or more hydrogen atoms in a substituent are substituted by one or more, identical or different halogen atoms. “Halogen” is as defined above. For example, “haloC₁-C₆alkyl” refers to a “C₁-C₆alkyl” in which one or more hydrogen atoms are substituted by one or more identical or different halogen atoms, where “C₁-C₆alkyl” is as defined above. Again for example, “haloC₁-C₆ alkoxy” refers to a “C₁-C₆ alkoxy” in which one or more hydrogen atoms are substituted by one or more identical or different halogen atoms, where “C₁-C₆ alkoxy” is as defined above.

The term “cyano” as used herein refers to the —CN group.

The term “hydroxy” as used herein refers to the —OH group.

The term “nitro” as used herein refers to the —NO₂ group.

The term “aryl” as used herein refers to a monovalent hydrocarbon group derived from a monocyclic or fused bicyclic or polycyclic ring system (in which at least one ring contains a fully conjugated π-electron system) with known aromatic characteristics. Fused aryl groups may include an aryl ring fused to a saturated or partially unsaturated carbocyclic or heterocyclic ring or to another aryl or heteroaryl ring, provided that the point of attachment to the parent molecule on this fused ring system is an atom of the aromatic radical of the ring system. Typically, an aryl group contains 6-12 carbon atoms (“C₆-C₁₂aryl”). Examples of an aryl group include, but are not limited to, phenyl, naphthyl, anthracenyl, phenanthrenyl, indanyl, indenyl and tetrahydronaphthyl.

As used herein, the term “arylene” refers to a divalent hydrocarbon group derived from a monocyclic or fused bicyclic or polycyclic ring system (in which at least one ring contains a fully conjugated π-electron system) with known aromatic characteristics. Typically, arylene contains 6-12 carbon atoms (“C₆-C₁₂ arylene”). Examples of an arylene group include, but are not limited to, phenylene, naphthylene, anthracenylene, phenanthrenylene, indanylene, indenylene and tetrahydronaphthylene.

The term “cycloalkyl” as used herein refers to a monovalent hydrocarbon group derived from a non-aromatic saturated carbocyclic ring system having a specified number of carbon atoms, which may monocyclic, spiro, bridged or fused bicyclic or polycyclic ring systems attached to the parent molecule through a carbon atom of the cycloalkyl ring. Typically, the cycloalkyl of the present disclosure contains 3-8 carbon atoms (“C₃-C₈ cycloalkyl”), preferably 3-7 carbon atoms (“C₃-C₇ cycloalkyl”) or 3-6 carbon atoms (“C₃-C₆ cycloalkyl”). Representative examples of a cycloalkyl group include cyclopropyl, cyclobutyl, cyclopentyl, cyclohexyl, cycloheptyl, cyclooctyl, and the like.

The term “cycloalkylene” as used herein refers to a divalent hydrocarbon group derived from a non-aromatic saturated carbocyclic ring system having a specified number of carbon atoms, which may be monocyclic, spiro, bridged or fused bicyclic or polycyclic ring systems attached to the parent molecule through carbon atom(s) of the cycloalkyl ring. Typically, the cycloalkylene of the present disclosure contains 3-8 carbon atoms (“C₃-C₈ cycloalkylene”), preferably 3-7 carbon atoms (“C₃-C₇ cycloalkylene”) or 3-6 carbon atoms (“C₃-C₆ cycloalkylene”). Representative examples of a cycloalkylene group include cyclopropylene, cyclobutylene, cyclopentylene, cyclohexyleneene, cycloheptylene, cyclooctylene, and the like.

The term “heteroatom” as used herein refers to N, O or S atom.

The term “heterocyclyl” as used herein refers to a monovalent group derived from a non-aromatic ring structure containing a specified number of carbon atoms and further including at least one heteroatom, preferably one to four heteroatoms, more preferably one or two heteroatoms as ring member, wherein the heteroatom refers to N, O or S atom and the S atom is optionally substituted with one or two oxo groups (i.e., S(O)_{q}, where q is 0, 1 or 2). Such heterocyclyl group may be partially unsaturated. Heterocyclyl groups include spiroc, bridged or fused rings formed with one or more other heterocyclic or carbocyclic rings, wherein such spiro, bridged or fused rings may themselves be saturated, partially unsaturated or aromatic, provided that the point of attachment to the parent molecule is an atom of the heterocyclic group of the ring system. Generally, a “heterocyclyl” group contains 3-12 ring atoms (i.e., 3-12 membered heterocyclyl), preferably 4-7 ring atoms (i.e., 4-7 membered heterocyclyl), and most preferably 5 or 6 ring atoms (i.e., 5 or 6 membered heterocyclyl), wherein the ring atoms include carbon and non-carbon heteroatoms. In certain embodiments, representative examples of a heterocyclyl group include azetidinyl, oxetanyl, thietanyl, pyrrolidinyl, imidazolidinyl, pyrazolidinyl, oxazolidinyl, thiazolidinyl, oxazolinyl, isoxazolinyl, pyrrolinyl, imidazolinyl, pyrazolinyl, thiazolinyl, piperidinyl, dihydropyridinyl, dihydropyrimidinyl, piperazinyl, dioxanyl, oxathianyl, azepanyl, diazepanyl, oxetanyl, tetrahydrofuranyl, tetrahydropyranyl, tetrahydrothiophenyl, tetrahydrothiopyranyl, morpholinyl, thiomorpholinyl, and the like.

The term “heterocyclylene” as used herein refers to a divalent group derived from a non-aromatic ring structure containing a specified number of carbon atoms and further including at least one heteroatom, preferably one to four heteroatoms, more preferably one or two heteroatoms as ring member, wherein the heteroatom refers to N, O or S atom and the S atom is optionally substituted with one or two oxo groups (i.e., S(O)_{q}, where q is 0, 1 or 2). Generally, a “heterocyclylene” group contains 3-12 ring atoms (i.e., 3-12 membered heterocyclylene), preferably 4-7 ring atoms (i.e., 4-7 membered heterocyclylene), and most preferably 5 or 6 ring atoms (i.e., 5 or 6 membered heterocyclylene), wherein the ring atoms include carbon and non-carbon heteroatoms. In certain embodiments, representative examples of a heterocyclylene group include azetidinylene, oxetanylene, thietanylene, pyrrolidinylene, imidazolidinylene, pyrazolidinylene, oxazolidinylene, thiazolidinylene, oxazolinylene, isoxazolinylene, pyrrolinylene, imidazolinylene, pyrazolinylene, thiazolinylene, piperidinylene, dihydropyridinylene, dihydropyrimidinylene, piperazinylene, dioxanylene, oxathianylene, azepanylene, diazepanylene, oxetanylene, tetrahydrofuranylene, tetrahydropyranylene, tetrahydrothiophenylene, tetrahydrothiopyranylene, morpholinylene, thiomorpholinylene, and the like.

The term “heteroaryl” as used herein refers to a monovalent group derived from an aromatic ring structure containing a specified number of carbon atoms and further including at least one heteroatom, preferably one to four heteroatoms, more preferably one or two heteroatoms as ring member, wherein the heteroatom refers to N, O or S atom. Generally, a heteroaryl group contains 5-12 ring atoms (“5-12membered heteroaryl”), preferably 5-10 ring atoms (“5-10membered heteroaryl”), more preferably 5 or 6 ring atoms (“5 or 6 membered heteroaryl”), wherein the ring atoms include carbon and non-carbon heteroatoms. A heteroaryl is attached to the parent molecule through a ring atom of the heteroaromatic ring, thus maintaining its aromaticity. A heteroaryl may also be fused to another aryl or heteroaryl ring, or to a saturated or partially unsaturated carbocyclic or heterocyclic ring, provided that the point of attachment to the basic molecule on this fused ring system is an atom of the heteroaromatic group of the ring system. In certain embodiments, representative examples of a heteroaryl group include furanyl, imidazolyl, isoxazolyl, thiazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiophenyl, triazolyl, triazinyl, benzimidazolyl, benzofuranyl, benzothiophenyl, benzoxadiazolyl, benzothiadiazolyl, benzothiazolyl, imidazopyridinyl, imidazopyrimidinyl, imidazopyridazinyl, cinnolinyl, furopyridinyl, indazolyl, indolyl, isoindolyl, isoquinolinyl, naphthyridinyl, purinyl, quinolinyl, thienopyridinyl, and the like.

The term “heteroarylene” as used herein refers to a divalent group derived from an aromatic ring structure containing a specified number of carbon atoms and further including at least one heteroatom, preferably one to four heteroatoms, more preferably one or two heteroatoms as ring member, wherein the heteroatom refers to N, O or S atom. Generally, a heteroarylene group contains 5-12 ring atoms (“5-12 membered heteroarylene”), preferably 5-10 ring atoms (“5-10 membered heteroarylene”), more preferably 5 or 6 ring atoms (“5 or 6 membered heteroarylene”), wherein the ring atoms include carbon and non-carbon heteroatoms. In certain embodiments, representative examples of a heteroarylene group include furanylene, imidazolylene, isoxazolylene, thiazolylene, isothiazolylene, oxadiazolylene, oxazolylene, pyridinylene, pyridazinylene, pyrimidinylene, pyrazinylene, pyrazolylene, pyrrolylene, tetrazolylene, thiadiazolylene, thiophenylene, triazolylene, triazinylene, benzimidazolylene, benzofuranylene, benzothiophenylene, benzoxadiazolylene, benzothiadiazolylene, benzothiazolylene, imidazopyridinylene, imidazopyrimidinylene, imidazopyridazinylene, cinnolinylene, furopyridinylene, indazolylene, indolylene, isoindolylene, isoquinolinylene, naphthyridinylene, purinylene, quinolinylene, thienopyridinylene, and the like.

The term “optionally” as used herein means that the situation described immediately following the term may or may not occur. For example, “X is optionally substituted with a substituent R” means “X is substituted with the substituent R or is not substituted with the substituent R”.

The term “pharmaceutically acceptable” as used herein refers to those compounds or pharmaceutical compositions, which within the scope of sound medical judgment are suitable for use in contact with tissue of a subject such as a human or other mammal without causing undue toxicity, irritation, allergic reactions or other problems, and simultaneously are commensurate with a reasonable benefit/risk ratio.

The term “pharmaceutically acceptable salt” as used herein refers to an acid or base addition salt formed from a compound of the present disclosure with a pharmaceutically acceptable acid or base, which retains the biological effectiveness and nature of the parent compound, i.e., the compound of the present disclosure. Such pharmaceutically acceptable salts include a salt formed from a compound of the present disclosure with an acid such as hydrochloric acid, hydrobromic acid, hydroiodic acid, phosphoric acid, phosphorous acid, nitric acid, sulfuric acid, sulfurous acid, formic acid, acetic acid, propionic acid, acrylic acid, caproic acid, caprylic acid, capric acid, methanesulfonic acid, ethanesulfonic acid, benzoic acid, benzenesulfonic acid, toluenesulfonic acid, citric acid, tartaric acid, maleic acid, and the like. Such pharmaceutically acceptable salts also include a salt formed from a compound of the present disclosure with sodium, calcium, ammonium, potassium, magnesium, manganese, iron, copper, zinc, aluminum, lithium; amino acids such as glycine and arginine; primary amine, secondary amine, tertiary amine, and cyclic amines such as piperidine, morpholine and piperazine.

The term “solvate” as used herein refers to a molecular complex comprising a compound of the present disclosure and one or more pharmaceutically acceptable solvent molecules (e.g., ethanol). When the solvent is water, the term “hydrate” is used.

The term “stereoisomer” as used herein refers to isomers resulting from differences in the spatial arrangement of atoms in a molecule. When asymmetric carbon atoms exist in a compound, enantiomers will exist; when a carbon-carbon double bond or cyclic structure exists in a compound, cis-trans isomers will exist. Included within the scope of this disclosure are all enantiomers, diastereomers, racemic isomers, cis-trans isomers, geometric isomers, epimers and mixture thereof of the compound of general formula (I).

The term “pharmaceutical composition” as used herein refers to a substance or material with specific medical use formed by combining a pharmaceutically active ingredient (in the context of the present disclosure, specifically a compound of the present disclosure or a pharmaceutically acceptable salt, hydrate, solvate and stereoisomer thereof) and a pharmaceutically acceptable excipient in a certain proportion. A pharmaceutical composition can be formulated into a pharmaceutically acceptable dosage form, such as tablets, powders (including sterile powders for injection), capsules, granules, solutions, syrups, suppositories, injections, patches, and the like by any conventional technique in the art.

A pharmaceutical composition of the present disclosure may be administered to a subject (e.g., a human or a non-human mammal) by any of a variety of routes of administration, including, for example, oral administration (e.g., in form of tablets, capsules, powders, granules); absorption (e.g., in form of suppositories, creams, or foams) via mucosal (e.g., sublingual, nasal, anal, rectal, or vaginal) route; parenteral administration (e.g., intramuscular, intravenous, intraperitoneal, subcutaneous or intrathecal injection); transdermal administration (e.g., as patches applied to the skin); and topical administration (e.g., as creams, ointments, or sprays applied to the skin, or as eye drops). The pharmaceutical compositions may also be formulated for administration by inhalation.

The term “pharmaceutically acceptable excipient” as used herein refers to an excipient (or carrier) that does not cause significant irritation to an organism and does not eliminate the biological activity and property of the administered compound. Any commonly used pharmaceutically acceptable excipient may be used, the selection of which will depend on factors such as the particular mode of administration, the effect of the excipient on solubility and stability, and the nature of the dosage form and is within the ordinary skill of those skilled in the art. Some examples of materials that can be used as pharmaceutically acceptable excipients include: starches, such as corn starch and potato starch; sugars, such as lactose, glucose, and sucrose; cellulose and its derivatives, such as ethyl cellulose, sodium carboxymethylcellulose and cellulose acetate; gelatin, gum arabic, guar gum, tragacanth; magnesium stearate, zinc stearate, talc; water, brine; oils such as peanut oil, cottonseed oil, olive oil, sesame oil, corn oil and soybean oil; alcohols such as ethanol, propylene glycol, glycerin, sorbitol, mannitol and polyethylene glycol; esters such as ethyl oleate and ethyl laurate; buffers such as sodium chloride, phosphate buffer solution, and the like.

The term “prevention/preventing” as used herein refers to preventing the appearance of a disease or the recurrence of a disease that has disappeared in a subject at risk of the disease.

The term “treatment/treating” as used herein refers to controlling, reducing or alleviating the pathological progression of a disease and prolonging the survival of a subject suffering a disease.

The term “programmed necrosis” as used herein refers to a necrosis pattern, which can be specifically inhibited by Necrostatin-1 (Nec-1), also known as “necroptosis”.

The term “RIP1” as used herein refers to “receptor interacting protein 1”, which contains a C-terminal death domain, an N-terminal serine/threonine kinase domain, and an intermediate domain that mediates nuclear factor κB activation.

The term “a disease that is at least partially mediated by RIP1 kinase” as used herein refers to a disease whose onset and progression are at least partially associated with abnormalities in RIP1 kinase activity. Exemplary diseases include, but are not limited to, Alzheimer's disease (AD), amyotrophic lateral sclerosis (ALS), multiple sclerosis (MS), Parkinson's disease (PD), systemic lupus erythematosus, inflammatory bowel disease and psoriasis, and the like.

The term “subject” as used herein refers to an individual animal to which a compound or pharmaceutical composition of the present disclosure is intended to be administered, including but not limited to humans and/or other primates (e.g., cynomolgus monkeys, rhesus monkeys); other mammals, such as horses, cattle, pigs, sheep, goats, cats, dogs; and poultry, such as chickens, ducks, geese, quails, and turkeys. Preferred subjects are humans.

The term “effective amount” as used herein refers to an amount sufficient to affect any one or more beneficial or desired symptoms of a disease, its complications, or intermediate pathological phenotypes exhibited during the development of the disease. The “effective amount”, in which a compound of the present disclosure, or a pharmaceutically acceptable salt, hydrate, solvate and stereoisomer thereof will be administrated, will depend on the species of the subject being treated, the severity of the disease, the frequency of administration, metabolic characteristics of the drug and other factors, and can be judged by the prescribing physician based on routine practice. In general, an effective amount will generally range from about 0.001 to about 100 mg/kg body weight/day, preferably from about 0.01 to about 50 mg/kg body weight/day (in a single dose or in divided doses). In some cases, dose levels below the lower end of the above range may be more than adequate, while in other cases, larger doses may be used without causing any deleterious side effects, where such larger doses are usually divided into several smaller doses for daily administration. It should be noted that all numerical ranges mentioned in this disclosure are meant to include both endpoints of the range, all integers within the range, and subranges formed by these integers.

### DETAILED DESCRIPTION OF THE INVENTION

The following specific embodiments are provided to enable those skilled in the art to more clearly understand the contents of the present disclosure. It should be noted that these embodiments are described for the purpose of illustration only and are not intended to limit the scope of protection of the present application.

The present disclosure relates generally to small molecule RIP1 kinase inhibitors and uses thereof. In some embodiments, the compounds of the present disclosure have excellent biological activity in inhibiting the programmed necrosis of cells. In some embodiments, the compounds of the present disclosure have excellent pharmacokinetic properties. In some embodiments, the compounds of the present disclosure have excellent blood-brain penetration. In some embodiments, the compounds of the present disclosure combine two or more of the above properties.

In a first aspect of the present disclosure there is provided a compound of general formula (I), or a pharmaceutically acceptable salt, hydrate, solvate and stereoisomer thereof:

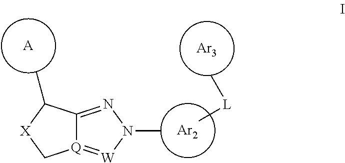

wherein each variable is as defined above.

In some embodiments, Ring A is C₆-C₁₂aryl, preferably phenyl or naphthyl, more preferably phenyl, wherein the above groups are substituted 5 with 0, 1 or 2 substituents independently selected from halogen, C₁-C₆alkyl, haloC₁-C₆alkyl, C₁-C₆alkoxy, haloC₁-C₆alkoxy, cyano and hydroxy, preferably substituted with 0, 1 or 2 substituents independently selected from halogen and cyano, more preferably substituted with 0, 1 or 2 substituents independently selected from F, Cl and cyano.

In some embodiments, Ring A is C₃-C₈ cycloalkyl, preferably C₄-C₇ cycloalkyl, more preferably cyclopentyl or cyclohexyl, still more preferably cyclohexyl, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy, preferably substituted with 0, 1 or 2 substituents independently selected from halogen and cyano, more preferably substituted with 0, 1 or 2 substituents independently selected from F, Cl and cyano.

In some embodiments, Ring A is 5 to 12 membered heteroaryl containing 1 or 2 heteroatoms independently selected from N, O and S, preferably 5 or 6 membered heteroaryl containing 1 or 2 heteroatoms independently selected from N, O and S, for example pyrrolyl, furanyl, thiophenyl, pyrazolyl, imidazolyl, oxazolyl, isoxazolyl, thiazolyl, isothiazolyl, oxadiazolyl, thiadiazolyl, tetrazolyl, triazolyl, pyridinyl, pyridazinyl, pyrimidinyl, pyrazinyl or triazinyl, more preferably 6 membered heteroaryl containing 1 or 2 N atoms, for example pyridinyl, pyridazinyl, pyrimidinyl or pyrazinyl, still more preferably pyridinyl, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy, preferably substituted with 0, 1 or 2 substituents independently selected from halogen and cyano, more preferably substituted with 0, 1 or 2 substituents independently selected from F, Cl and cyano.

In some embodiments, Ring Ar₂ is C₆-C₁₂ arylene, preferably phenylene or naphthylene, more preferably phenylene, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy, preferably substituted with 0 or 1 C₁-C₆alkyl groups, more preferably substituted with 0 or 1 substituents independently selected from methyl and ethyl.

In some embodiments, Ring Ar₂ is C₃-C₈ cycloalkylene, preferably C₄-C₇ cycloalkylene, more preferably cyclopentylene or cyclohexylene, still more preferably cyclopentylene, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy, preferably substituted with 0 or 1 C₁-C₆alkyl groups, more preferably substituted with 0 or 1 substituents independently selected from methyl and ethyl.

In some embodiments, Ring Ar₂ is 3 to 12 membered heterocyclylene containing 1 or 2 heteroatoms independently selected from N, O and S, preferably 5 or 6 membered heterocyclylene containing 1 or 2 heteroatoms independently selected from N, O and S, for example pyrrolidinylene, tetrahydrofuranylene, tetrahydrothiophenylene, pyrazolidinylene, imidazolidinylene, oxazolidinylene, isoxazolidinylene, thiazolidinylene, isothiazolidinylene, pyrrolinylene, dihydrofuranylene, dihydrothiophenylene, pyrazolinylene, imidazolinylene, oxazolinylene, isoxazolinylene, thiazolinylene, isothiazolinylene, piperidinylene, piperazinylene, morpholinylene, thiomorpholinylene, dihydropyridinylene, dihydropyrazinylene, dihydropyrimidinylene, dihydropyridazinylene, more preferably 6 membered heterocyclylene containing 1 N atom, for example piperidinylene, dihydropyridinylene, still more preferably dihydropyridinylene, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy and further substituted with one ═O group, preferably substituted with 0 or 1 C₁-C₆alkyl groups and further substituted with one ═O group, more preferably substituted with 0 or 1 substituents independently selected from methyl and ethyl and further substituted with one ═O group.

In some embodiments, Ring Ar₂ is 5 to 12 membered heteroarylene containing 1 or 2 heteroatoms independently selected from N, O and S, preferably 5 or 6 membered heteroarylene containing 1 or 2 heteroatoms independently selected from N, O and S, for example pyrrolylene, furanylene, thiophenylene, pyrazolylene, imidazolylene, oxazolylene, isoxazolylene, thiazolylene, isothiazolylene, oxadiazolylene, thiadiazolylene, tetrazolylene, triazolylene, pyridinylene, pyridazinylene, pyrimidinylene, pyrazinylene or triazinylene, more preferably 6 membered heteroarylene containing 1 or 2 N atoms, for example pyridinylene, pyridazinylene, pyrimidinylene or pyrazinylene, still more preferably pyridinylene or pyrimidinylene, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy, preferably substituted with 0 or 1 C₁-C₆alkyl groups, more preferably substituted with 0 or 1 substituents independently selected from methyl and ethyl.

In some embodiments, Ring Ar₃ is C₆-C₁₂aryl, preferably phenyl or naphthyl, more preferably phenyl, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano, hydroxy, —NR¹R² and —C(═O)NR¹R², preferably substituted with 0, 1 or 2 substituents independently selected from halogen, C₁-C₆alkyl, C₁-C₆alkoxy, hydroxy, —NH₂ and —C(═O)NH₂, more preferably substituted with 0, 1 or 2 substituents independently selected from chloro, methyl, methoxy, hydroxy, —NH₂ and —C(═O)NH₂.

In some embodiments, Ring Ar₃ is 3 to 12 membered heterocyclyl containing 1 or 2 heteroatoms independently selected from N, O and S, preferably 5 or 6 membered heterocyclyl containing 1 or 2 heteroatoms independently selected from N, O and S, for example pyrrolidinyl, tetrahydrofuranyl, tetrahydrothiophenyl, pyrazolidinyl, imidazolidinyl, oxazolidinyl, isoxazolidinyl, thiazolidinyl, isothiazolidinyl, pyrrolinyl, dihydrofuranyl, dihydrothiophenyl, pyrazolinyl, imidazolinyl, oxazolinyl, isoxazolinyl, thiazolinyl, isothiazolinyl, piperidinyl, piperazinyl, morpholinyl, thiomorpholinyl, dihydropyridinyl, dihydropyrazinyl, dihydropyrimidinyl, dihydropyridazinyl, more preferably 6 membered heterocyclyl containing 1 or 2 N atoms, for example dihydropyridinyl, dihydropyrazinyl, dihydropyrimidinyl, dihydropyridazinyl, still more preferably dihydropyrimidinyl, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano, hydroxy, —NR¹R² and —C(═O)NR¹R² and further substituted with one ═O group, preferably substituted with 0, 1 or 2 substituents independently selected from halogen, C₁-C₆alkyl, C₁-C₆alkoxy, hydroxy, —NH₂ and —C(═O)NH₂ and further substituted with one ═O group, more preferably substituted with 0, 1 or 2 substituents independently selected from chloro, methyl, methoxy, hydroxy, —NH₂ and —C(═O)NH₂ and further substituted with one ═O group.

In some embodiments, Ring Ar₃ is 5 to 12 membered heteroaryl containing 1 or 2 heteroatoms independently selected from N, O and S, for example pyrrolyl, furanyl, thiophenyl, pyrazolyl, imidazolyl, oxazolyl, isoxazolyl, thiazolyl, isothiazolyl, oxadiazolyl, thiadiazolyl, tetrazolyl, triazolyl, pyridinyl, pyridazinyl, pyrimidinyl, pyrazinyl, triazinyl, benzimidazolyl, benzofuranyl, benzothiophenyl, benzoxadiazolyl, benzothiadiazolyl, benzothiazolyl, imidazopyridinyl, imidazopyrimidinyl, imidazopyridazinyl, cinnolinyl, furopyridinyl, indazolyl, indolyl, isoindolyl, isoquinolinyl, naphthyridinyl, purinyl, quinolinyl, thienopyridinyl, preferably pyrazolyl, imidazolyl, thiazolyl, pyridinyl, pyridazinyl, pyrimidinyl, pyrazinyl, imidazopyridinyl or imidazopyridazinyl, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano, hydroxy, —NR¹R² and —C(═O)NR¹R², preferably substituted with 0, 1 or 2 substituents independently selected from halogen, C₁-C₆alkyl, C₁-C₆alkoxy, hydroxy, —NH₂ and —C(═O)NH₂, more preferably substituted with 0, 1 or 2 substituents independently selected from chloro, methyl, methoxy, hydroxy, —NH₂ and —C(═O)NH₂.

In some embodiments, L is C₁-C₆alkylene or C₂-C₆alkynylene, preferably methylene, ethylene, n-propylene or ethynylene, more preferably ethylene or ethynylene.

In some embodiments, X is CR¹R² or O, preferably CH₂ or O.

In some embodiments, Q is C and the dashed line between Q and W represents a double bond and at the same time W is CR³ or N.

In some embodiments, Q is N and the dashed line between Q and W represents a single bond and at the same time W is —C(═O)—.

In some embodiments, W is CR³, preferably CH, CF, CCl, CBr, C(CH₃) or C(C₂H₅).

In some embodiments, W is N.

In some embodiments, each of R¹ and R² is independently H.

In some embodiments, each of R¹ and R² is independently C₁-C₆alkyl, preferably methyl or ethyl.

In some embodiments, R³ is H, halogen or C₁-C₆alkyl, preferably H, F, Cl, Br, methyl or ethyl, more preferably H, Cl or methyl.

In some embodiments, the present disclosure provides a compound of general formula (I), or a pharmaceutically acceptable salt, hydrate, solvate and stereoisomer thereof:

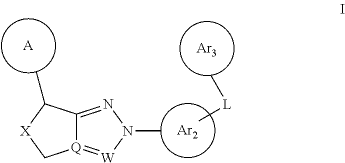

wherein,

Ring A is phenyl or naphthyl; C₄-C₇ cycloalkyl; or 5 or 6 membered heteroaryl containing 1 or 2 heteroatoms independently selected from N, O and S, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy;

Ring Ar₂ is phenylene or naphthylene; C₄-C₇cycloalkylene; 5 or 6 membered heterocyclylene containing 1 or 2 heteroatoms independently selected from N, O and S; or 5 or 6 membered heteroarylene containing 1 or 2 heteroatoms independently selected from N, O and S, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy and the heterocyclylene is further substituted with one ═O group;

Ring Ar₃ is phenyl or naphthyl; 5 or 6 membered heterocyclyl containing 1 or 2 heteroatoms independently selected from N, O and S; or 5 to 12 membered heteroaryl containing 1 or 2 heteroatoms independently selected from N, O and S, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano, hydroxy, —NR¹R² and —C(═O)NR¹R² and the heterocyclyl is further substituted with one ═O group;

L is selected from C₁-C₆alkylene or C₂-C₆alkynylene;

X is CR¹R² or O;

Q is C or N;

W is CR³, —C(═O)— or N;

the dashed line between Q and W represents a single bond or a double bond, provided that when Q is C, the dashed line between Q and W represents a double bond and at the same time W is CR³ or N, and when Q is N, the dashed line between Q and W represents a single bond and at the same time W is —C(═O)—;

each of R¹ and R² is independently H, methyl or ethyl; and

R³ is H, halogen or C₁-C₆alkyl.

In some embodiments, the present disclosure provides a compound of general formula (I), or a pharmaceutically acceptable salt, hydrate, solvate and stereoisomer thereof:

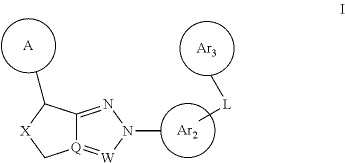

wherein,

Ring A is phenyl; cyclopentyl or cyclohexyl; or 6 membered heteroaryl containing 1 or 2 N atoms, wherein the above groups are substituted with 0, 1 or 2 substituents independently selected from halogen and cyano;

Ring Ar₂ is phenylene; cyclopentylene or cyclohexylene; 6 membered heterocyclylene containing 1 N atom; or 6 membered heteroarylene containing 1 or 2 N atoms, wherein the above groups are substituted with 0 or 1 C₁-C₆alkyl groups and the heterocyclylene is further substituted with one ═O group;

Ring Ar₃ is phenyl; 6 membered heterocyclyl containing 1 or 2 N atoms; or pyrazolyl, imidazolyl, thiazolyl, pyridinyl, pyridazinyl, pyrimidinyl, pyrazinyl, imidazopyridinyl or imidazopyridazinyl, wherein the above groups are substituted with 0, 1 or 2 substituents independently selected from halogen, C₁-C₆alkyl, C₁-C₆alkoxy, hydroxy, —NH₂ and —C(═O)NH₂ and the heterocyclyl is further substituted with one ═O group;

L is selected from methylene, ethylene, n-propylene or ethynylene;

X is CH₂ or 0;

Q is C or N;

W is CR³, —C(═O)— or N;

the dashed line between Q and W represents a single bond or a double bond, provided that when Q is C, the dashed line between Q and W represents a double bond and at the same time W is CR³ or N, and when Q is N, the dashed line between Q and W represents a single bond and at the same time W is —C(═O)—; and

R³ is H, F, Cl, Br, methyl or ethyl.

In some embodiments, the present disclosure provides a compound of general formula (I), or a pharmaceutically acceptable salt, hydrate, solvate and stereoisomer thereof:

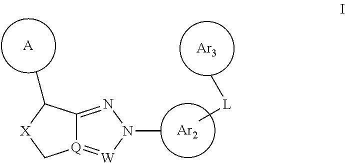

wherein,

Ring A is phenyl; cyclohexyl; or pyridinyl, wherein the above groups are substituted with 0, 1 or 2 substituents independently selected from F, Cl and cyano;

Ring Ar₂ is phenylene; cyclopentylene; dihydropyridinylene; or pyridinylene or pyrimidinylene, wherein the above groups are substituted with 0 or 1 substituents independently selected from methyl and ethyl and the dihydropyridinylene is further substituted with one ═O group;

Ring Ar₃ is phenyl; dihydropyrimidinyl; or pyrazolyl, imidazolyl, thiazolyl, pyridinyl, pyridazinyl, pyrimidinyl, pyrazinyl, imidazopyridinyl or imidazopyridazinyl, wherein the above groups are substituted with 0, 1 or 2 substituents independently selected from chloro, methyl, methoxy, hydroxy, —NH₂ and —C(═O)NH₂ and the dihydropyrimidinyl is further substituted with one ═O group;

L is selected from ethylene or ethynylene;

X is CH₂ or O;

Q is C or N;

W is CR³, —C(═O)— or N;

the dashed line between Q and W represents a single bond or a double bond, provided that when Q is C, the dashed line between Q and W represents a double bond and at the same time W is CR³ or N, and when Q is N, the dashed line between Q and W represents a single bond and at the same time W is —C(═O)—; and

R³ is H, Cl or methyl.

In some embodiments, the present disclosure provides compounds as exemplified in Examples 1-66, or a pharmaceutically acceptable salt, hydrate, solvate and stereoisomer thereof.

In some embodiments, the present disclosure provides compounds selected from those in the following table, or a pharmaceutically acceptable salt, hydrate, solvate and stereoisomer thereof:

| | |
|---|---|
| 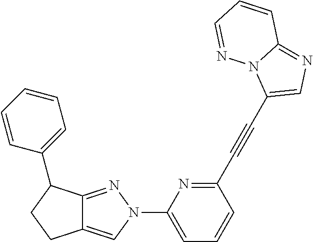 [SMILES: C(#Cc1cnc2cccnn12)c1cccc(-n2cc3c(n2)C(c2ccccc2)CC3)n1] | Compound 1 |
| 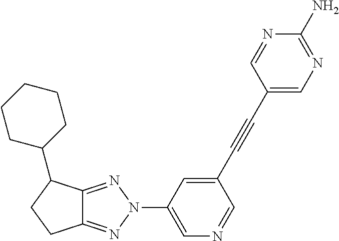 [SMILES: Nc1ncc(C#Cc2cncc(-n3nc4c(n3)C(C3CCCCC3)CC4)c2)cn1] | Compound 2 |
| 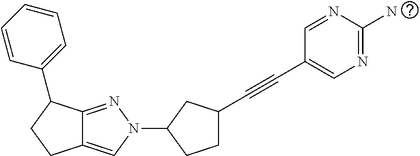 [SMILES: Nc1ncc(C#CC2CCC(n3cc4c(n3)C(c3ccccc3)CC4)C2)cn1] | Compound 3 |
| 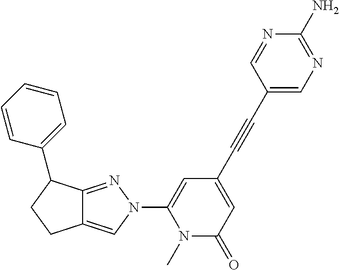 [SMILES: Cn1c(-n2cc3c(n2)C(c2ccccc2)CC3)cc(C#Cc2cnc(N)nc2)cc1=O] | Compound 4 |
| 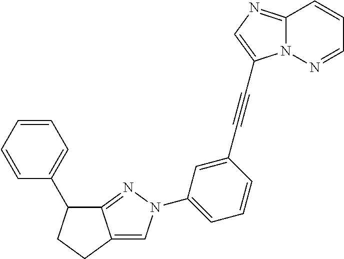 [SMILES: C(#Cc1cnc2cccnn12)c1cccc(-n2cc3c(n2)C(c2ccccc2)CC3)c1] | Compound 5 |
| 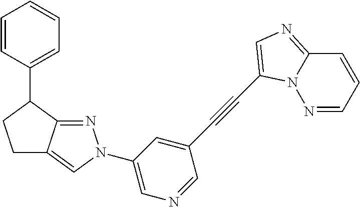 [SMILES: C(#Cc1cnc2cccnn12)c1cncc(-n2cc3c(n2)C(c2ccccc2)CC3)c1] | Compound 6 |
| 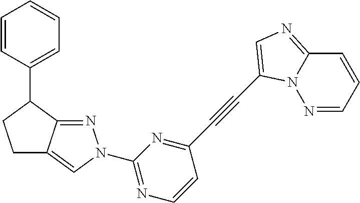 [SMILES: C(#Cc1cnc2cccnn12)c1ccnc(-n2cc3c(n2)C(c2ccccc2)CC3)n1] | Compound 7 |
| 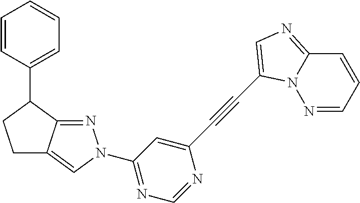 [SMILES: C(#Cc1cnc2cccnn12)c1cc(-n2cc3c(n2)C(c2ccccc2)CC3)ncn1] | Compound 8 |
| 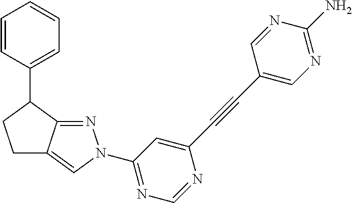 [SMILES: Nc1ncc(C#Cc2cc(-n3cc4c(n3)C(c3ccccc3)CC4)ncn2)cn1] | Compound 9 |
| 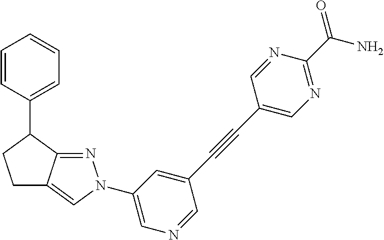 [SMILES: NC(=O)c1ncc(C#Cc2cncc(-n3cc4c(n3)C(c3ccccc3)CC4)c2)cn1] | Compound 10 |
| 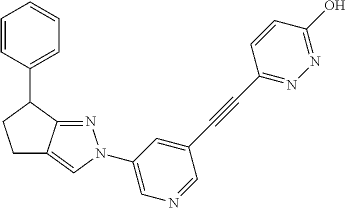 [SMILES: Oc1ccc(C#Cc2cncc(-n3cc4c(n3)C(c3ccccc3)CC4)c2)nn1] | Compound 11 |
| 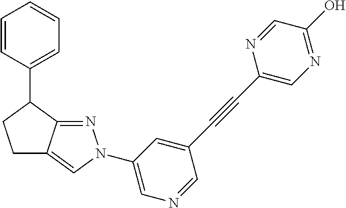 [SMILES: Oc1cnc(C#Cc2cncc(-n3cc4c(n3)C(c3ccccc3)CC4)c2)cn1] | Compound 12 |
| 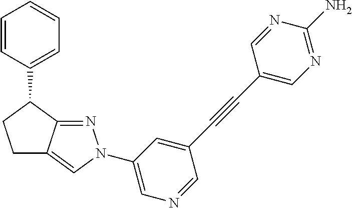 [SMILES: Nc1ncc(C#Cc2cncc(-n3cc4c(n3)[C@@H](c3ccccc3)CC4)c2)cn1] | Compound 13 |
| 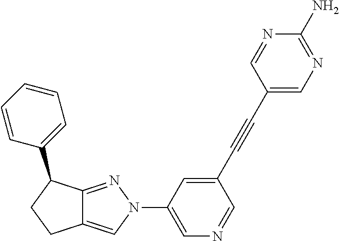 [SMILES: Nc1ncc(C#Cc2cncc(-n3cc4c(n3)[C@H](c3ccccc3)CC4)c2)cn1] | Compound 14 |
| 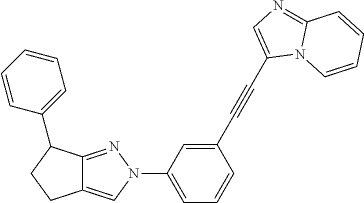 [SMILES: C(#Cc1cnc2ccccn12)c1cccc(-n2cc3c(n2)C(c2ccccc2)CC3)c1] | Compound 15 |
| 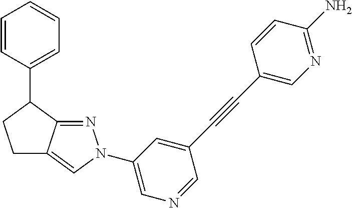 [SMILES: Nc1ccc(C#Cc2cncc(-n3cc4c(n3)C(c3ccccc3)CC4)c2)cn1] | Compound 16 |
| 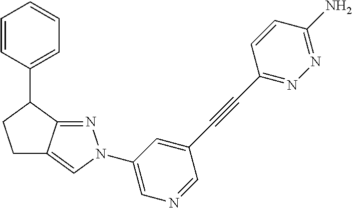 [SMILES: Nc1ccc(C#Cc2cncc(-n3cc4c(n3)C(c3ccccc3)CC4)c2)nn1] | Compound 17 |
| 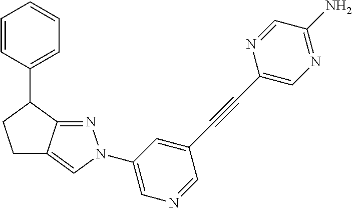 [SMILES: Nc1cnc(C#Cc2cncc(-n3cc4c(n3)C(c3ccccc3)CC4)c2)cn1] | Compound 18 |
| 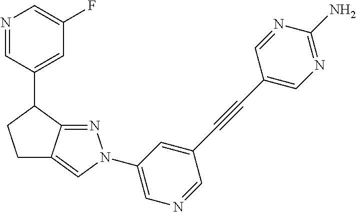 [SMILES: Nc1ncc(C#Cc2cncc(-n3cc4c(n3)C(c3cncc(F)c3)CC4)c2)cn1] | Compound 19 |
| 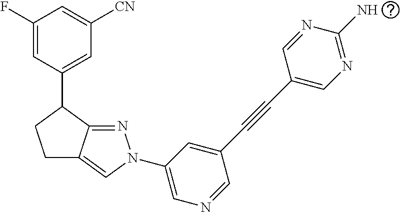 [SMILES: N#Cc1cc(F)cc(C2CCc3cn(-c4cncc(C#Cc5cnc(N)nc5)c4)nc32)c1] | Compound 20 |
| 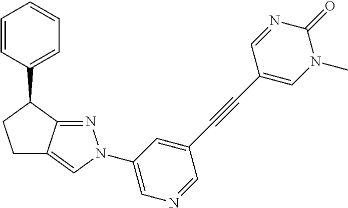 [SMILES: Cn1cc(C#Cc2cncc(-n3cc4c(n3)[C@H](c3ccccc3)CC4)c2)cnc1=O] | Compound 21 |
| 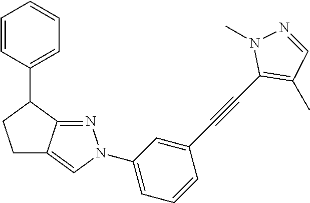 [SMILES: Cc1cnn(C)c1C#Cc1cccc(-n2cc3c(n2)C(c2ccccc2)CC3)c1] | Compound 22 |
| 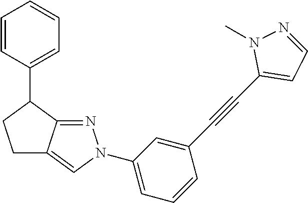 [SMILES: Cn1nccc1C#Cc1cccc(-n2cc3c(n2)C(c2ccccc2)CC3)c1] | Compound 23 |
| 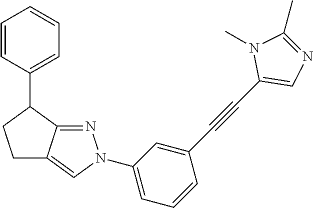 [SMILES: Cc1ncc(C#Cc2cccc(-n3cc4c(n3)C(c3ccccc3)CC4)c2)n1C] | Compound 24 |
| 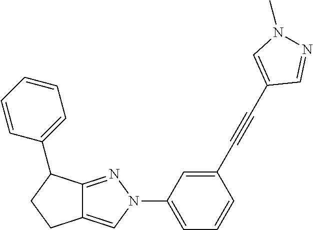 [SMILES: Cn1cc(C#Cc2cccc(-n3cc4c(n3)C(c3ccccc3)CC4)c2)cn1] | Compound 25 |
| 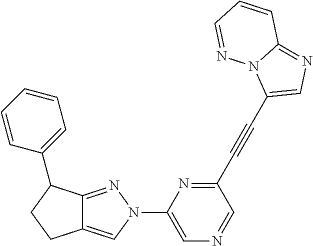 [SMILES: C(#Cc1cnc2cccnn12)c1cncc(-n2cc3c(n2)C(c2ccccc2)CC3)n1] | Compound 26 |
| 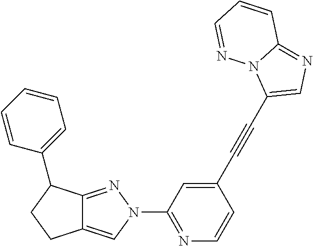 [SMILES: C(#Cc1cnc2cccnn12)c1ccnc(-n2cc3c(n2)C(c2ccccc2)CC3)c1] | Compound 27 |
| 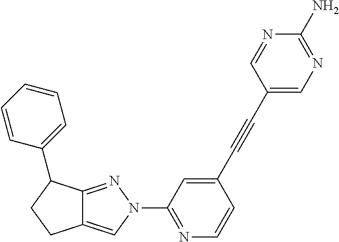 [SMILES: Nc1ncc(C#Cc2ccnc(-n3cc4c(n3)C(c3ccccc3)CC4)c2)cn1] | Compound 28 |
| 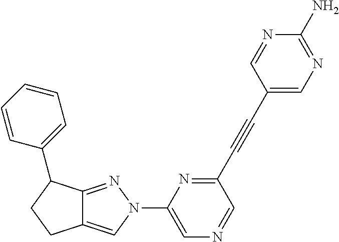 [SMILES: Nc1ncc(C#Cc2cncc(-n3cc4c(n3)C(c3ccccc3)CC4)n2)cn1] | Compound 29 |
| 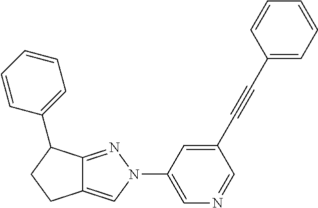 [SMILES: C(#Cc1cncc(-n2cc3c(n2)C(c2ccccc2)CC3)c1)c1ccccc1] | Compound 30 |
| 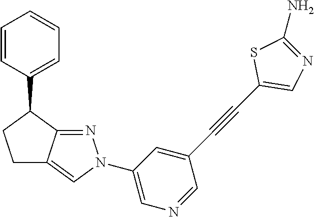 [SMILES: Nc1ncc(C#Cc2cncc(-n3cc4c(n3)[C@H](c3ccccc3)CC4)c2)s1] | Compound 31 |
| 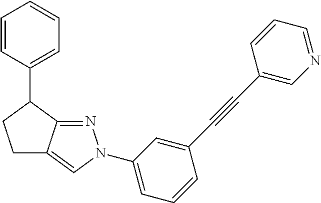 [SMILES: C(#Cc1cccc(-n2cc3c(n2)C(c2ccccc2)CC3)c1)c1cccnc1] | Compound 32 |
| 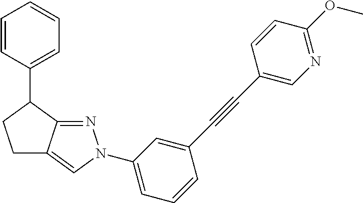 [SMILES: COc1ccc(C#Cc2cccc(-n3cc4c(n3)C(c3ccccc3)CC4)c2)cn1] | Compound 33 |
| 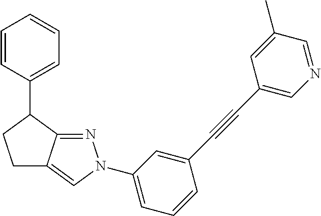 [SMILES: Cc1cncc(C#Cc2cccc(-n3cc4c(n3)C(c3ccccc3)CC4)c2)c1] | Compound 34 |
| 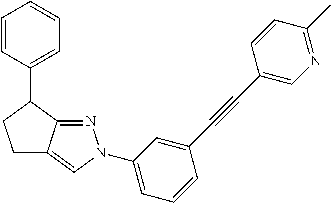 [SMILES: Cc1ccc(C#Cc2cccc(-n3cc4c(n3)C(c3ccccc3)CC4)c2)cn1] | Compound 35 |
| 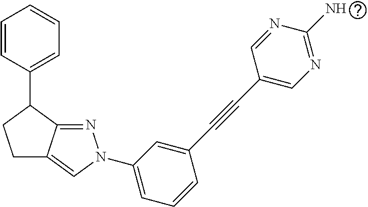 [SMILES: Nc1ncc(C#Cc2cccc(-n3cc4c(n3)C(c3ccccc3)CC4)c2)cn1] | Compound 36 |
| 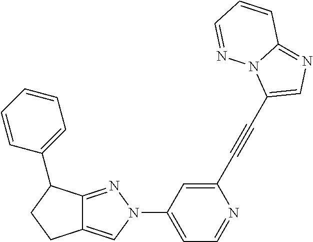 [SMILES: C(#Cc1cnc2cccnn12)c1cc(-n2cc3c(n2)C(c2ccccc2)CC3)ccn1] | Compound 37 |
| 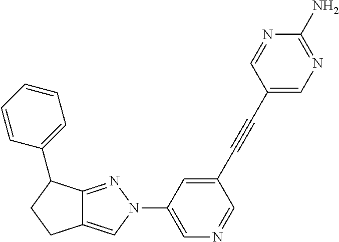 [SMILES: Nc1ncc(C#Cc2cncc(-n3cc4c(n3)C(c3ccccc3)CC4)c2)cn1] | Compound 38 |
| 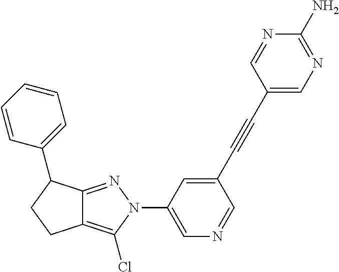 [SMILES: Nc1ncc(C#Cc2cncc(-n3nc4c(c3Cl)CCC4c3ccccc3)c2)cn1] | Compound 39 |
| 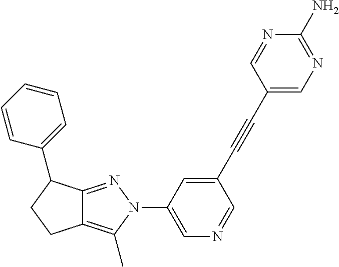 [SMILES: Cc1c2c(nn1-c1cncc(C#Cc3cnc(N)nc3)c1)C(c1ccccc1)CC2] | Compound 40 |
| 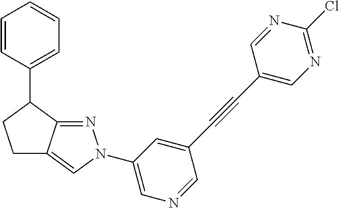 [SMILES: Clc1ncc(C#Cc2cncc(-n3cc4c(n3)C(c3ccccc3)CC4)c2)cn1] | Compound 41 |
| 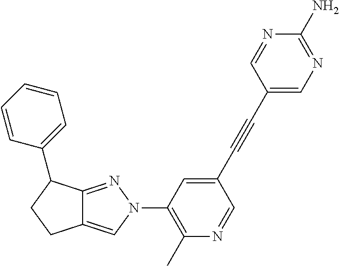 [SMILES: Cc1ncc(C#Cc2cnc(N)nc2)cc1-n1cc2c(n1)C(c1ccccc1)CC2] | Compound 42 |
| 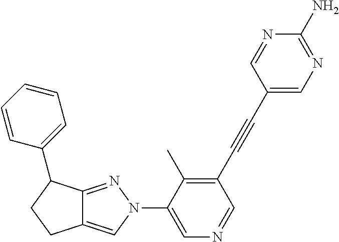 [SMILES: Cc1c(C#Cc2cnc(N)nc2)cncc1-n1cc2c(n1)C(c1ccccc1)CC2] | Compound 43 |
| 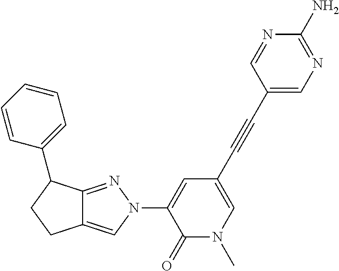 [SMILES: Cn1cc(C#Cc2cnc(N)nc2)cc(-n2cc3c(n2)C(c2ccccc2)CC3)c1=O] | Compound 44 |
| 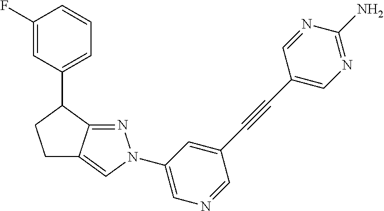 [SMILES: Nc1ncc(C#Cc2cncc(-n3cc4c(n3)C(c3cccc(F)c3)CC4)c2)cn1] | Compound 45 |
| 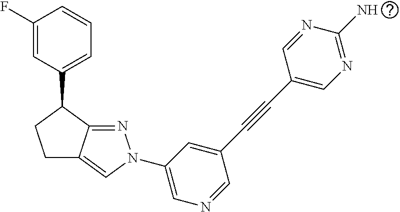 [SMILES: Nc1ncc(C#Cc2cncc(-n3cc4c(n3)[C@H](c3cccc(F)c3)CC4)c2)cn1] | Compound 46 |
| 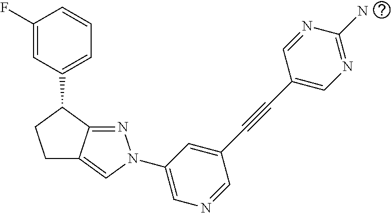 [SMILES: Nc1ncc(C#Cc2cncc(-n3cc4c(n3)[C@@H](c3cccc(F)c3)CC4)c2)cn1] | Compound 47 |
| 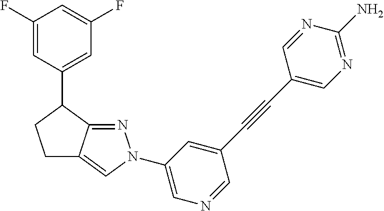 [SMILES: Nc1ncc(C#Cc2cncc(-n3cc4c(n3)C(c3cc(F)cc(F)c3)CC4)c2)cn1] | Compound 48 |
| 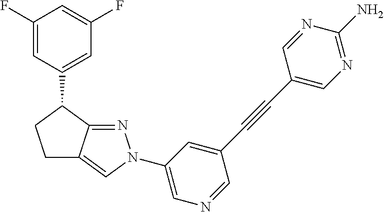 [SMILES: Nc1ncc(C#Cc2cncc(-n3cc4c(n3)[C@@H](c3cc(F)cc(F)c3)CC4)c2)cn1] | Compound 49 |
| 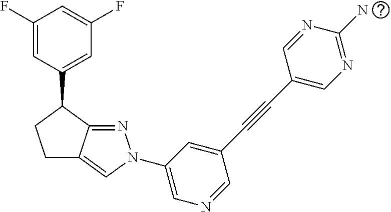 [SMILES: Nc1ncc(C#Cc2cncc(-n3cc4c(n3)[C@H](c3cc(F)cc(F)c3)CC4)c2)cn1] | Compound 50 |
| 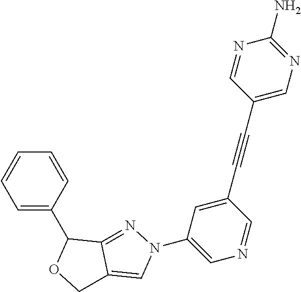 [SMILES: Nc1ncc(C#Cc2cncc(-n3cc4c(n3)C(c3ccccc3)OC4)c2)cn1] | Compound 51 |
| 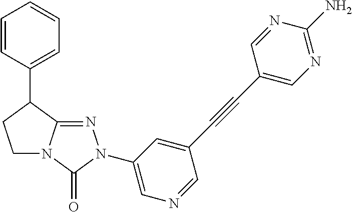 [SMILES: Nc1ncc(C#Cc2cncc(-n3nc4n(c3=O)CCC4c3ccccc3)c2)cn1] | Compound 52 |
| 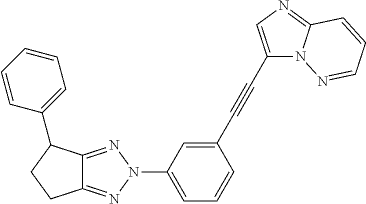 [SMILES: C(#Cc1cnc2cccnn12)c1cccc(-n2nc3c(n2)C(c2ccccc2)CC3)c1] | Compound 53 |
| 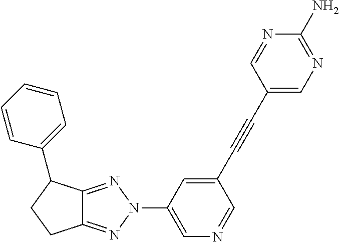 [SMILES: Nc1ncc(C#Cc2cncc(-n3nc4c(n3)C(c3ccccc3)CC4)c2)cn1] | Compound 54 |
| 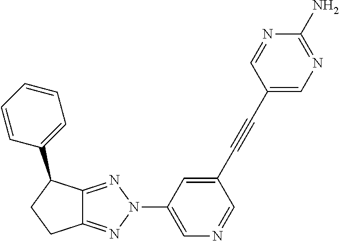 [SMILES: Nc1ncc(C#Cc2cncc(-n3nc4c(n3)[C@H](c3ccccc3)CC4)c2)cn1] | Compound 55 |
| 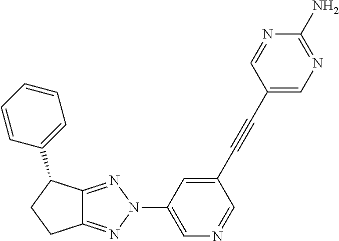 [SMILES: Nc1ncc(C#Cc2cncc(-n3nc4c(n3)[C@@H](c3ccccc3)CC4)c2)cn1] | Compound 56 |
| 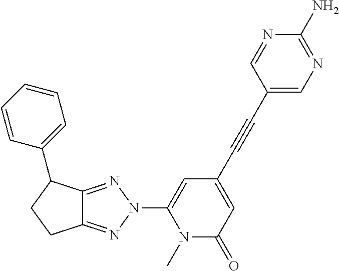 [SMILES: Cn1c(-n2nc3c(n2)C(c2ccccc2)CC3)cc(C#Cc2cnc(N)nc2)cc1=O] | Compound 57 |
| 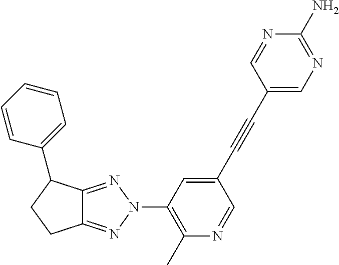 [SMILES: Cc1ncc(C#Cc2cnc(N)nc2)cc1-n1nc2c(n1)C(c1ccccc1)CC2] | Compound 58 |
| 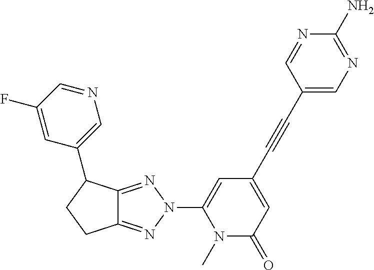 [SMILES: Cn1c(-n2nc3c(n2)C(c2cncc(F)c2)CC3)cc(C#Cc2cnc(N)nc2)cc1=O] | Compound 59 |
| 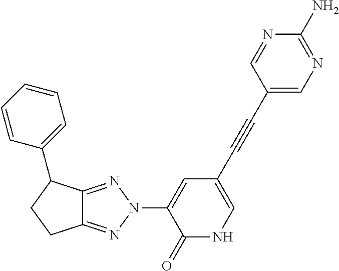 [SMILES: Nc1ncc(C#Cc2c[nH]c(=O)c(-n3nc4c(n3)C(c3ccccc3)CC4)c2)cn1] | Compound 60 |
| 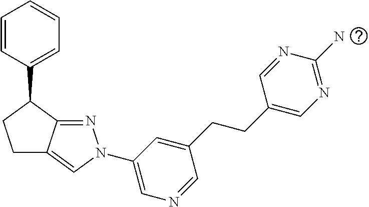 [SMILES: Nc1ncc(CCc2cncc(-n3cc4c(n3)[C@H](c3ccccc3)CC4)c2)cn1] | Compound 61 |
| 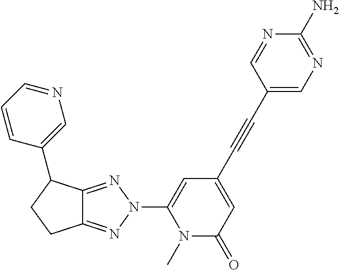 [SMILES: Cn1c(-n2nc3c(n2)C(c2cccnc2)CC3)cc(C#Cc2cnc(N)nc2)cc1=O] | Compound 62 |
| 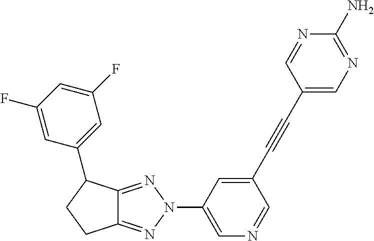 [SMILES: Nc1ncc(C#Cc2cncc(-n3nc4c(n3)C(c3cc(F)cc(F)c3)CC4)c2)cn1] | Compound 63 |
| 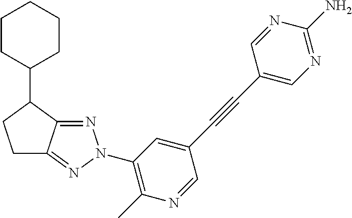 [SMILES: Cc1ncc(C#Cc2cnc(N)nc2)cc1-n1nc2c(n1)C(C1CCCCC1)CC2] | Compound 64 |
| 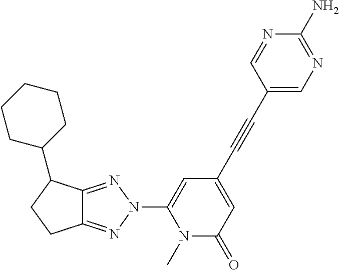 [SMILES: Cn1c(-n2nc3c(n2)C(C2CCCCC2)CC3)cc(C#Cc2cnc(N)nc2)cc1=O] | Compound 65 |
| 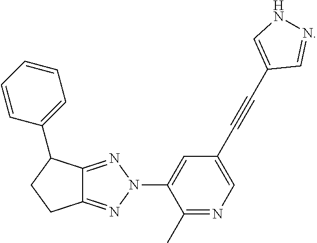 [SMILES: Cc1ncc(C#Cc2cn[nH]c2)cc1-n1nc2c(n1)C(c1ccccc1)CC2] | Compound 66 |

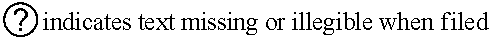

In a second aspect of the present disclosure there is provided a pharmaceutical composition, comprising a compound of general formula (I), or a pharmaceutically acceptable salt, hydrate, solvate and stereoisomer thereof and a pharmaceutically acceptable excipient. In some embodiments according to this aspect, the pharmaceutical composition contains two or more pharmaceutically acceptable excipients. In some embodiments according to this aspect, the pharmaceutical composition is in the form of a pharmaceutically acceptable dosage form.

In a third aspect of the present disclosure there is provided a compound of general formula (I), or a pharmaceutically acceptable salt, hydrate, solvate and stereoisomer thereof, or a pharmaceutical composition comprising a compound of general formula (I), or a pharmaceutically acceptable salt, hydrate, solvate and stereoisomer thereof and a pharmaceutically acceptable excipient, for use in treating or preventing a disease.

In a fourth aspect of the present disclosure there is provided a compound of general formula (I), or a pharmaceutically acceptable salt, hydrate, solvate and stereoisomer thereof, or a pharmaceutical composition comprising a compound of general formula (I), or a pharmaceutically acceptable salt, hydrate, solvate and stereoisomer thereof and a pharmaceutically acceptable excipient, for use in inhibiting programmed necrosis, inhibiting RIP1 kinase, or treating or preventing a disease that is at least partially mediated by RIP1 kinase.

In a fifth aspect of the present disclosure there is provided use of a compound of general formula (I), or a pharmaceutically acceptable salt, hydrate, solvate and stereoisomer thereof, or a pharmaceutical composition comprising a compound of general formula (I), or a pharmaceutically acceptable salt, hydrate, solvate and stereoisomer thereof and a pharmaceutically acceptable excipient for inhibiting programmed necrosis, inhibiting RIP1 kinase, or treating or preventing a disease that is at least partially mediated by RIP1 kinase.

In a sixth aspect of the present disclosure there is provided use of a compound of general formula (I), or a pharmaceutically acceptable salt, hydrate, solvate and stereoisomer thereof, or a pharmaceutical composition comprising a compound of general formula (I), or a pharmaceutically acceptable salt, hydrate, solvate and stereoisomer thereof and a pharmaceutically acceptable excipient in manufacture of a medicament for inhibiting programmed necrosis, inhibiting RIP1 kinase, or treating or preventing a disease that is at least partially mediated by RIP1 kinase.

In a seventh aspect of the present disclosure there is provided a method for inhibiting programmed necrosis, inhibiting RIP1 kinase, or treating or preventing a disease that is at least partially mediated by RIP1 kinase, which method comprises administering to a subject in need thereof a therapeutically effective amount of compound of general formula (I), or a pharmaceutically acceptable salt, hydrate, solvate and stereoisomer thereof, or a pharmaceutical composition comprising a compound of general formula (I), or a pharmaceutically acceptable salt, hydrate, solvate and stereoisomer thereof and a pharmaceutically acceptable excipient.

### General Synthesis Schemes

By reading the following general synthesis routes, those skilled in the art can readily understand the general procedures for preparing the compounds of the present disclosure, wherein Schemes 1-3 illustrates the preparation process of a compound of general formula (I), in which the linker L is C₂-C₆alkynylene (specifically, a compound of general formula (II), a compound of general formula (III) or a compound of general formula (IV)), and Schemes 4-5 illustrates the preparation process of a compound of general formula (I), in which the linker L is C₁-C₆alkylene (specifically, a compound of general formula (V) and a compound of general formula (VI)).

It is noted that in the following schemes, variables not defined in detail have the same meaning as the corresponding variables in the compound of general formula (I) described previously.

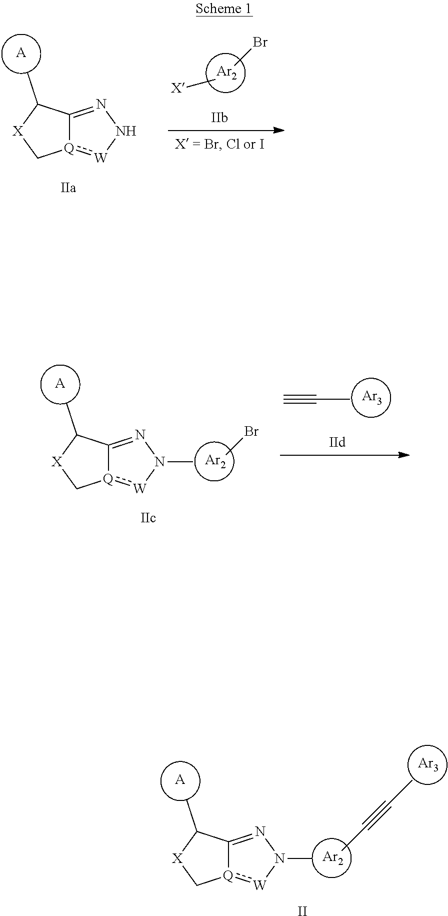

As described in Scheme 1, when W is CR³ or —C(═O)—, the compound of general formula (II) can be prepared from a compound of general formula (IIa) in two steps. In the first step, a coupling reaction or nucleophilic substitution reaction occurs between the compound of general formula (IIa) and a dihalide of general formula (IIb). Specifically, in a polar aprotic solvent (such as acetonitrile), in the presence of a copper catalyst (such as cuprous oxide), a ligand (such as (E)-picolinaldehyde oxime), an inorganic base (such as cesium carbonate) and a tertiary amine (such as triethylamine), the reaction is performed at 85° C. for 16 hours; or in a polar aprotic solvent (such as 1,4-dioxane), in the presence of a copper catalyst (such as cuprous iodide), a ligand (such as (1R,2R)—N,N′-dimethyl-1,2-cyclohexanediamine) and an inorganic base (such as potassium phosphate), the reaction is performed under microwave at 120° C. for 3 hours; or in a polar aprotic solvent (such as DMF), in the presence of an inorganic base (such as cesium carbonate), the reaction is performed at 85° C. for 16 hours to produce a halide of general formula (IIc). In the second step, the compound of general formula (II) can be prepared by reacting the halide of general formula (IIc) with an alkyne of general formula (IId). Specifically, in a polar aprotic solvent (such as DMF), in the presence of a copper catalyst (such as cuprous iodide) and a tertiary amine (such as triethylamine), in the presence of a palladium catalyst (such as 1,1′-bis(diphenylphosphino)ferrocene dichloropalladium), the reaction is performed under microwave at 100° C. for 30 minutes or in the presence of a palladium catalyst (such as bis(triphenylphosphine)dichloropalladium), the reaction is performed at 100° C. for 16 hours or at 120° C. for 2 hours to produce the compound of general formula (II).

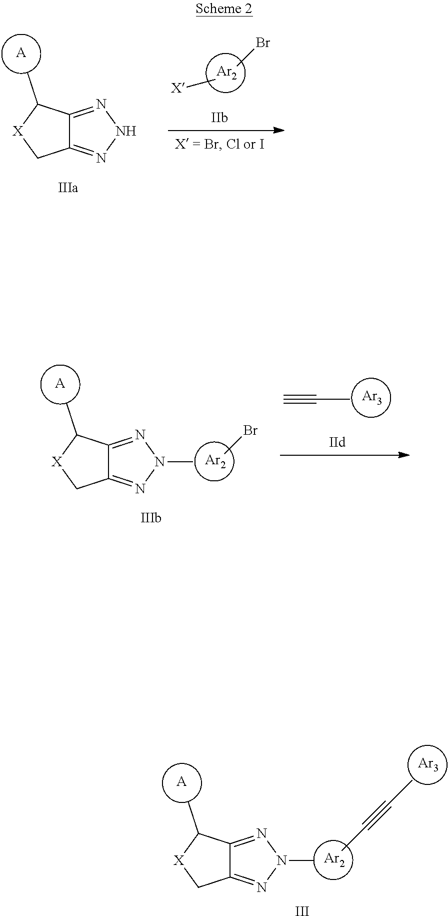

As described in Scheme 2, the compound of general formula (III) can be prepared from a triazole compound of general formula (IIIa) in two steps. In the first step, a coupling reaction or nucleophilic substitution reaction occurs between the triazole compound of general formula (IIIa) and a dihalide of general formula (IIb). Specifically, in a polar aprotic solvent (such as DMSO), in the presence of a copper catalyst (such as cuprous chloride), a ligand (such as L-proline) and an inorganic base (such as potassium carbonate), the reaction is performed under microwave at 160° C. for 0.5 hours; or in a polar aprotic solvent (such as DMF), in the presence of a copper catalyst (such as cuprous iodide), a ligand (such as (1R,2R)—N,N′-dimethyl-1,2-cyclohexanediamine) and an inorganic base (such as cesium carbonate), the reaction is performed at 110° C. for 16.0 hours; in a polar aprotic solvent (such as acetonitrile), in the presence of an inorganic base (such as potassium carbonate), the reaction is performed under microwave at 125° C. for 30 minutes, to produce a halide of general formula (IIIb). In the second step, the compound of general formula (III) can be prepared by reacting the halide of general formula (IIIb) with an alkyne of general formula (IId). Specifically, in a polar aprotic solvent (such as DMF), in the presence of a copper catalyst (such as cuprous iodide) and a tertiary amine (such as triethylamine), in the presence of a palladium catalyst (such as 1,1′-bis(diphenylphosphino)ferrocene dichloropalladium), the reaction is performed under microwave at 100° C. for 30 minutes or in the presence of a palladium catalyst (such as bis(triphenylphosphine)dichloropalladium), the reaction is performed at 100° C. for 16 hours to produce the compound of general formula (III).

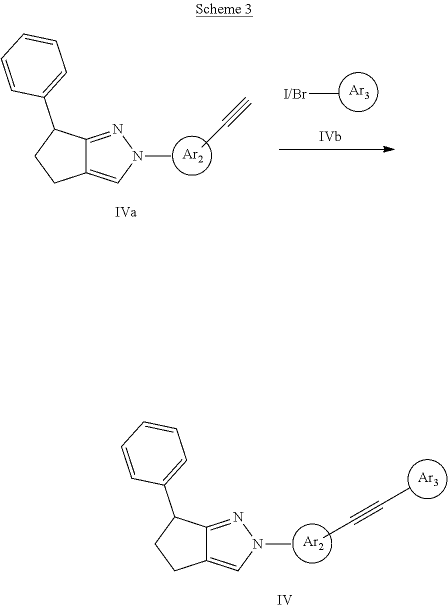

As described in Scheme 3, the compound of general formula (IV) can be prepared by reacting an alkyne of general formula (IVa) with a halide of general formula (IVb). In a polar aprotic solvent (such as DMF or THF), in the presence of a palladium catalyst (such as 1,1′-bis(diphenylphosphino)ferrocene dichloropalladium or tetra(triphenylphosphine)palladium), a copper catalyst (such as cuprous iodide) and a tertiary amine (such as triethylamine), the reaction is performed by heating at 100° C. for 1 hour or the reaction is performed under microwave at 80° C. for 30 minutes.

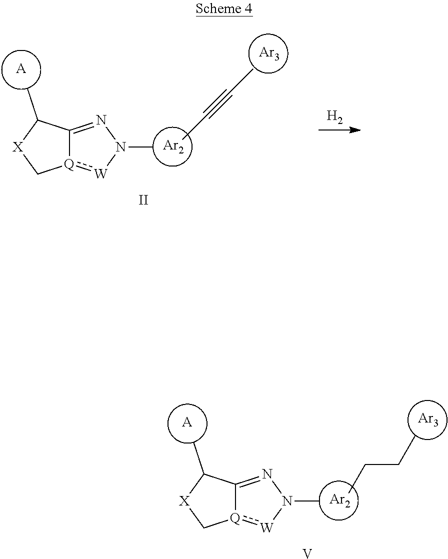

As described in Scheme 4, the compound of general formula (V) can be prepared from the compound of general formula (II) by the hydrogenation addition. The reaction is performed in a polar protic solvent (such as a mixed solution of methanol and water (volume ratio 5/1)) in the presence of a palladium catalyst (such as palladium acetate) at room temperature under 18 psi for 30 minutes.

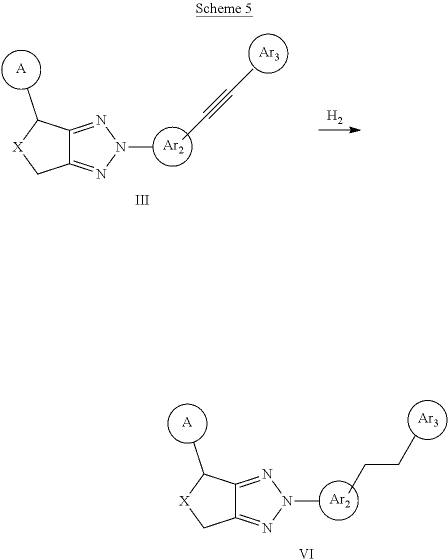

As described in Scheme 5, the compound of general formula (VI) can be prepared from the compounds of general formula (III) by the hydrogenation addition. The reaction is performed in a polar protic solvent (such as a mixed solution of methanol and water (volume ratio 5/1)) in the presence of a palladium catalyst (such as palladium acetate) at room temperature under 18 psi for 30 minutes.

### PREPARATION EXAMPLES

The compounds of the present disclosure can be readily prepared according to the following specific examples or variations thereof using readily available starting materials, chemical reagents and conventional synthetic procedures.

### Example 1: Synthesis of 3-((6-(6-phenyl-5,6-dihydrocyclopenta[c]pyrazol-2 (4H)-yl)pyridin-2-yl)ethynyl)imidazo[1,2-b]pyridazine

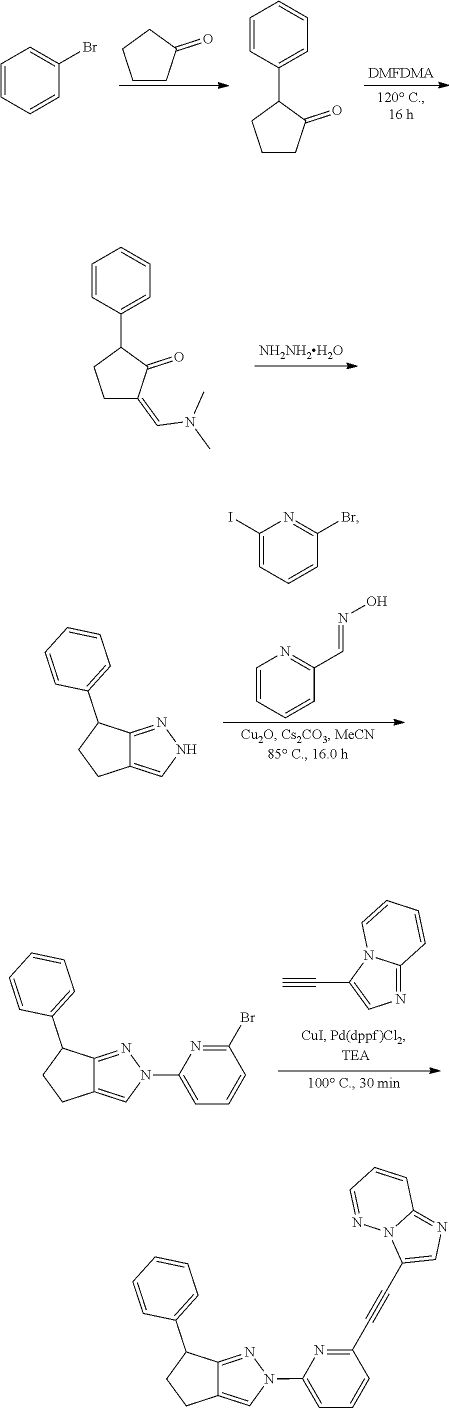

### Step 1: Synthesis of 2-phenylcyclopentan-1-one

Bromobenzene (12.132 g, 0.077 mol), cyclopentanone (5 g, 0.059 mol), tert-octylamine (2.304 g, 0.0178 mol), tri(ortho-methylphenyl)phosphine (0.905 g, 2.973 mmol), palladium acetate (0.333 g, 1.486 mmol), sodium acetate (4.87 g, 0.059 mol) and tetrahydropyrrole (1.268 g, 0.0178 mol) were mixed into 1,4-dioxane (100 mL). Under nitrogen protection, the mixture was reacted at 110° C. for 16 hours. LCMS detection indicated the completion of the reaction, and the solvent was removed by rotary-evaporation to dryness. Water (50 mL) was added. The resulting mixture was extracted with ethyl acetate (3×150 mL). The organic phases were combined, washed with brine (20 mL), dried over anhydrous Na₂SO₄, and filtered. The filtrate was concentrated under reduced pressure. The residue was purified with silica gel column (petroleum ether/ethyl acetate=10/1→7/1) to produce 2-phenylcyclopentan-1-one (6.2 g) as a brown oil in a yield of 65.7%. LC-MS (m/z): 161.3 [M+H]⁺.

### Step 2: Synthesis of (Z)-2-((dimethylamino)methylene)-5-phenylcyclopentan-1-one

2-phenylcyclopentan-1-one (800 mg, 4.99 mmol) and N,N-dimethylformamide dimethyl acetal (2.97 g, 24.97 mmol) were dissolved in N,N-dimethylformamide (5 mL). Under nitrogen protection, the mixture was dissolved by stirring at room temperature and reacted at 110° C. for 16 hours. LCMS detection indicated the completion of the reaction. The reaction mixture was cooled to room temperature, diluted with water (20 mL), and extracted with ethyl acetate (3×15 mL). The combined organic phases were washed with brine (30 mL), dried over anhydrous Na₂SO₄, filtered, and then concentrated under reduced pressure to produce (Z)-2-((dimethylamino)methylene)-5-phenylcyclopentan-1-one (1.0 g) as a light yellow oil in a yield of 93.0%, which was directly used for the reaction in the next step. LC-MS (m/z): 216.3 [M+H]⁺.

### Step 3: Synthesis of 6-phenyl-2,4,5,6-tetrahydrocyclopenta[c]pyrazole

(Z)-2-((dimethylamino)methylene)-5-phenylcyclopentan-1-one (500 mg, 2.322 mmol) was dissolved in ethanol (4.2 mL). 85% hydrazine hydrate (0.167 mL) was added to the above reaction mixture. The reaction mixture was reacted under reflux for 16 hours. The reaction mixture was rotary-evaporated to dryness under reduced pressure, and the residue was purified by reverse-phase flash chromatography (spherical C₁₈ column 40-60 μm, 40 g; 40% acetonitrile) to produce 6-phenyl-2,4,5,6-tetrahydrocyclopenta[c]pyrazole (298 mg) as a light yellow solid in a yield of 69.8%. LC-MS (m/z): 185.2 [M+H]⁺.

### Step 4: Synthesis of 2-(6-bromopyridin-2-yl)-6-phenyl-2,4,5,6-tetrahydrocyclopenta[c]pyrazole

6-phenyl-2,4,5,6-tetrahydrocyclopenta[c]pyrazole (150 mg, 0.81 mmol) and 2-bromo-6-iodopyridine (208 mg, 0.73 mmol) were mixed into acetonitrile (5 mL). At room temperature and under nitrogen protection, (E)-picolinaldehyde oxime (20 mg, 0.16 mmol), cuprous oxide (12 mg, 0.08 mmol) and cesium carbonate (663 mg, 2.04 mmol) were added. The resulting mixture was further stirred at 85° C. for 16 hours. LCMS detection indicated the completion of the reaction. The reaction mixture was cooled to room temperature, and diluted with water (10 mL). The resulting mixture was extracted with ethyl acetate (3×15 mL). The organic phases were combined, washed with brine (20 mL), dried over anhydrous Na₂SO₄, and filtered. The filtrate was concentrated under reduced pressure. The residue was purified by reverse-phase flash chromatography (spherical C₁₈ column 40-60 um, 40 g; 43% acetonitrile) to produce 2-(6-bromopyridin-2-yl)-6-phenyl-2,4,5,6-tetrahydrocyclopenta[c]pyrazole (12 mg) as a white solid in a yield of 4.3%. LC-MS (m/z): 341.2 [M+H]⁺. ¹H NMR (400 MHz, chloroform-d) δ 8.24-8.21 (m, 1H), 7.82 (dd, J=8.2, 0.8 Hz, 1H), 7.55 (t, J=7.9 Hz, 1H), 7.33-7.29 (m, 4H), 7.27-7.23 (m, 2H), 4.34 (dd, J=8.3, 6.8 Hz, 1H), 3.00-2.70 (m, 3H), 2.41 (ddt, J=12.7, 8.3, 6.8 Hz, 1H).

### Step 5: Synthesis of 3-(6-(6-phenyl-5,6-dihydrocyclopenta[c]pyrazol-2 (4H)-yl)pyridin-2-yl)ethynyl)imidazo[1,2-b]pyridazine

2-(6-bromopyridin-2-yl)-6-phenyl-2,4,5,6-tetrahydrocyclopenta[c]pyrazole (56 mg, 0.16 mmol) and 3-ethynylimidazo[1,2-b]pyridazine (28 mg, 0.19 mmol) were dissolved in N,N-dimethylformamide (3 mL). Under the nitrogen atmosphere Pd(dppf)Cl₂ (12 mg, 0.02 mmol), CuI (3 mg, 0.02 mmol) and triethylamine (1 mL) were added. The mixture was heated and reacted under microwave irradiation at 100° C. for 30 minutes and cooled to room temperature. At room temperature, the reaction mixture was diluted with (5 mL). The resulting mixture was filtered. The filter cake was washed with ethyl acetate (3 mL). The filtrate was extracted with ethyl acetate (3×5 mL). The organic layers were combined, washed with brine (10 mL), dried over anhydrous Na₂SO₄, and filtered. The filtrate was concentrated under reduced pressure. The residue was purified by reverse-phase flash chromatography (spherical C₁₈ column: 40-60 μm, 40 g; 55% acetonitrile) to produce 3-(6-(6-phenyl-5,6-dihydrocyclopenta[c]pyrazol-2 (4H)-yl)pyridin-2-yl)ethynyl)imidazo[1,2-b]pyridazine (12 mg) as a light yellow solid in a yield of 18.1%. LC-MS (m/z): 403.4 [M+H]⁺. ¹H NMR (400 MHz, chloroform-d) δ 8.54 (d, J=4.3 Hz, 1H), 8.36 (d, J=1.2 Hz, 1H), 8.16 (s, 1H), 8.08 (d, J=9.1 Hz, 1H), 7.91 (dd, J=8.4, 0.9 Hz, 1H), 7.74 (dd, J=8.4, 7.4 Hz, 1H), 7.45 (dd, J=7.5, 0.9 Hz, 1H), 7.32 (d, J=4.3 Hz, 4H), 7.26-7.16 (m, 2H), 4.36 (dd, J=8.3, 6.8 Hz, 1H), 2.97 (dtd, J=12.8, 8.2, 4.6 Hz, 1H), 2.91-2.71 (m, 2H), 2.43 (ddt, J=12.6, 8.3, 6.7 Hz, 1H).

The following intermediates were synthesized with reference to the synthesis of 6-phenyl-2,4,5,6-tetrahydrocyclopenta[c]pyrazole.

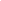

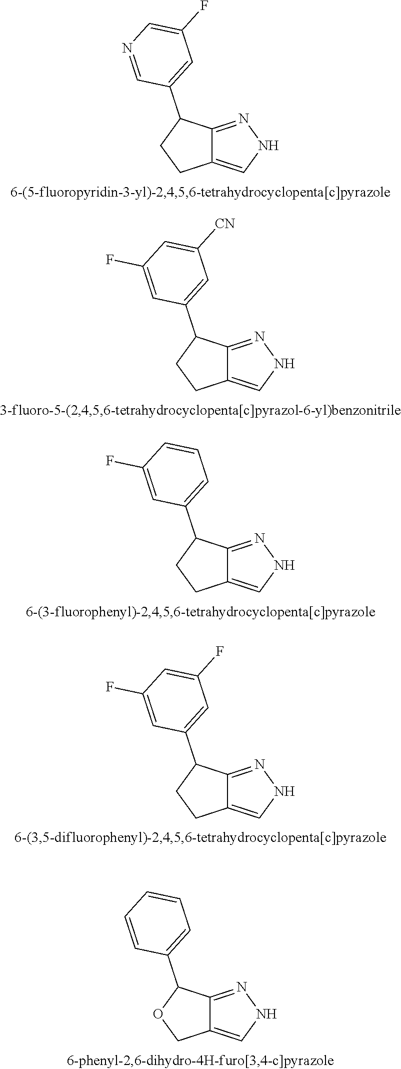 [SMILES: Fc1cncc(C2CCc3c[nH]nc32)c1 ; N#Cc1cc(F)cc(C2CCc3c[nH]nc32)c1 ; Fc1cccc(C2CCc3c[nH]nc32)c1 ; Fc1cc(F)cc(C2CCc3c[nH]nc32)c1 ; c1ccc(C2OCc3c[nH]nc32)cc1]

### Example 2: Synthesis of 5-((5-(4-cyclohexyl-5,6-dihydrocyclopenta[d][1,2,3]triazol-2 (4H)-yl)pyridin-3-yl)ethynyl)pyrimidin-2-amine

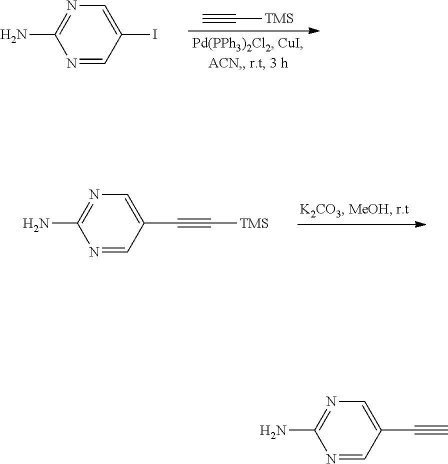

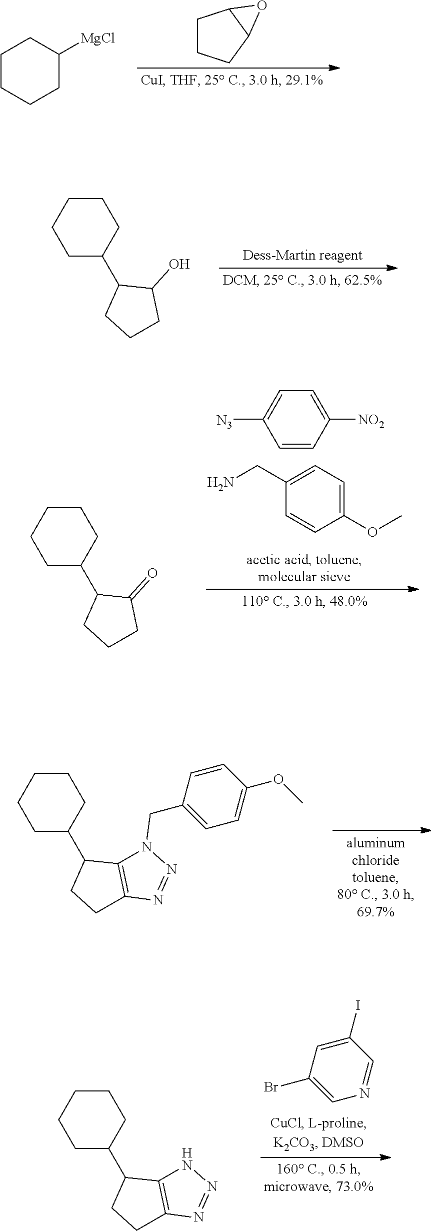

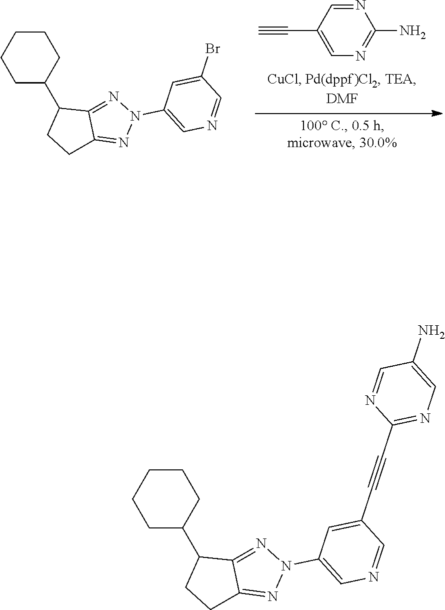

### Step 1: Synthesis of 5-((trimethylsilyl)ethynyl)pyrimidin-2-amine

5-iodopyrimidin-2-amine (25.0 g, 113 mmol), ethynyltrimethylsilane (35 ml, 241 mmol), triethylamine (93 ml, 658 mmol), Pd(PPh₃)₂Cl₂ (3.9 g, 5.5 mmol), and cuprous iodide (1.1 g, 5.5 mmol) were mixed into acetonitrile (900 mL). The mixture was stirred overnight under nitrogen protection at room temperature. The solvent was removed by rotary-evaporation to dryness under reduced pressure. The residue was directly used for the reaction in the next step.

### Step 2: Synthesis of 5-ethynylpyrimidin-2-amine

To the above residue were added methanol (600 mL) and potassium carbonate (152 g, 109.7 mmol). The resulting mixture was stirred for 2 hours at room temperature. Activated carbon (50 g) was added to the reaction mixture. The reaction mixture continued to be stirred for 15 minutes, and then filtered with diatomaceous earth. The filtrate was concentrated to 400 mL, and the resulting precipitate was filtered. The filtrate was concentrated to a thick paste, and placed in 10% methanol/water (150 mL) and stood at room temperature for 20 minutes. Then the formed solid was filtered to produce 5-ethynylpyrimidin-2-amine (9 g) as a light-yellow solid in a two-step yield of 66.6%. LC-MS (m/z): 120.2 [M+H]⁺.

### Step 3: Synthesis of 2-cyclohexylcyclopentan-1-ol

Cyclohexylmagnesium chloride (147.7 g, 1.04 mol), and cuprous iodide (19.7 g, 0.10 mol) were mixed into tetrahydrofuran (500 mL). The mixture was reacted under nitrogen protection at 25° C. for 0.5 hours. Then 6-oxabicyclo[3.1.0]hexane (87.36 g, 1.04 mol) was dissolved in tetrahydrofuran (100 mL), and slowly added dropwise to the reaction system at 25° C. Under nitrogen protection, the reaction system continued to be stirred for 2.5 hours. LCMS detection indicated the completion of the reaction, and the solvent was removed by rotary-evaporation to dryness. The resulting mixture was diluted with ethyl acetate (1000 mL), washed with brine (500 mL), dried over anhydrous Na₂SO₄, and filtered. The filtrate was concentrated under reduced pressure. The residue was purified with silica gel column (petroleum ether/ethyl acetate=1/0→10/1) to produce 2-cyclohexylcyclopentan-1-ol (49.0 g) as a brown oil in a yield of 29.1%. LC-MS (m/z): 169.3 [M+H]⁺.

### Step 4: Synthesis of 2-cyclohexylcyclopentan-1-one

2-cyclohexylcyclopentan-1-ol (49.0 g, 0.29 mol) was dissolved in dichloromethane (1000 mL). Dess-Martin oxidizer (123.0 g, 0.29 mol) was added to the system in portions at 25° C. Under nitrogen protection, the reaction system was stirred and reacted at 25° C. for 3.0 hours. LCMS detection indicated the completion of the reaction. The reaction system was diluted with water (500 mL). Sodium carbonate powder was slowly added to adjust the pH of the solution to 8. Then sodium thiosulfate (72.0 g, 0.29 mol) was added to the reaction system. The reaction system was stirred at 25° C. for 0.5 hours, and separated into two phases. The organic phase was collected, and washed with brine (2×300 mL), dried over anhydrous Na₂SO₄, filtered, and then concentrated under reduced pressure. The residue was purified with silica gel column (petroleum ether/ethyl acetate=1/0-98/1) to produce 2-cyclohexylcyclopentan-1-one 30.0 g) as a brown oil in a yield of 62.5%. LC-MS (m/z): 167.3 [M+H]⁺.

### Step 5: Synthesis of 6-cyclohexyl-1-(4-methoxybenzyl)-1,4,5,6-tetrahydrocyclopenta[d][1,2,3]triazole

2-cyclohexylcyclopentan-1-one (1.50 g, 9.03 mmol), 1-azido-4-nitrobenzene (1.50 g, 9.14 mmol), (4-methoxyphenyl)methanamine (1.73 g, 12.65 mmol), 4 Å molecular sieve (1.5 g), and glacial acetic acid (0.5 mL) were dissolved in toluene (15 mL). Under nitrogen protection, the mixture was reacted under reflux for 3.0 hours. The reaction mixture was rotary-evaporated to dryness under reduced pressure, and the residue was purified by reverse-phase flash chromatography (spherical C₁₈ column 40-60 μm, 120 g; 70% acetonitrile) to produce 6-cyclohexyl-1-(4-methoxybenzyl)-1,4,5,6-tetrahydrocyclopenta[d][1,2,3]triazole (1.4 g) as a brown oil in a yield of 48.0%. LC-MS (m/z): 312.2 [M+H]⁺.

### Step 6: Synthesis of 6-cyclohexyl-1,4,5,6-tetrahydrocyclopenta[d][1,2,3]triazole

6-cyclohexyl-1-(4-methoxybenzyl)-1,4,5,6-tetrahydrocyclopenta[d][1,2,3]triazole (1.40 g, 4.50 mmol) and anhydrous aluminium chloride (2.0 g, 15.00 mmol) were mixed into toluene (15 mL). Under nitrogen protection, the mixture was reacted at 80° C. under stirring for 3.0 hours. LCMS detection indicated the completion of the reaction. The reaction mixture was cooled to room temperature, and diluted with ice-water (50 mL). The resulting mixture was extracted with ethyl acetate (2×50 mL). The organic phases were combined, washed with brine (50 mL), dried over anhydrous Na₂SO₄, and filtered. The filtrate was concentrated under reduced pressure. The residue was purified with silica gel column (petroleum ether/ethyl acetate=1/0→3/1) to produce 6-cyclohexyl-1,4,5,6-tetrahydrocyclopenta[d][1,2,3]triazole (600 mg) as a brown oil in a yield of 69.7%. LC-MS (m/z): 192.3 [M+H]⁺.

### Step 7: Synthesis of 2-(5-bromopyridin-3-yl)-4-cyclohexyl-2,4,5,6-tetrahydrocyclopenta[d][1,2,3]triazole

6-cyclohexyl-1,4,5,6-tetrahydrocyclopenta[d][1,2,3]triazole (300 mg, 1.56 mmol) and 3-bromo-5-iodopyridine (550 mg, 1.93 mmol) were dissolved in dimethyl sulfoxide (10 mL). Under the nitrogen atmosphere, cuprous chloride (20 mg, 0.20 mmol), L-proline (37 mg, 0.32 mmol) and anhydrous potassium carbonate (450 mg, 3.20 mmol) were added. The mixture was heated and reacted at 160° C. under microwave irradiation for 30 minutes, and cooled to room temperature. At room temperature, the reaction mixture was diluted with water (30 mL). The resulting mixture was filtered. The filter cake was washed with ethyl acetate (10 mL). The filtrate was extracted with ethyl acetate (3×30 mL). The organic layers were combined, washed with brine (50 mL), dried over anhydrous Na₂SO₄, and filtered. The filtrate was concentrated under reduced pressure. The residue was purified by reverse-phase flash chromatography (spherical C₁₈ column: 40-60 μm, 40 g; 90% acetonitrile) to produce 2-(5-bromopyridin-3-yl)-4-cyclohexyl-2,4,5,6-tetrahydrocyclopenta[d][1,2,3]triazole (394 mg) as a light-yellow oil in a yield of 73.0%. LC-MS (m/z): 347.3 [M+H]⁺.

### Step 8: Synthesis of 5-((5-(4-cyclohexyl-5,6-dihydrocyclopenta[d][1,2,3]triazole-2 (4H-yl)pyridin-3-yl)ethynyl)pyrimidin-2-amine

2-(5-bromopyridin-3-yl)-4-cyclohexyl-2,4,5,6-tetrahydrocyclopenta[d][1,2,3]triazole (300 mg, 0.86 mmol) and 5-ethynylpyrimidin-2-amine (100 mg, 0.84 mmol) were dissolved in N,N-dimethylformamide (10 mL). Under the nitrogen atmosphere, cuprous iodide (20 mg, 0.10 mmol), [1,1′-bis(diphenylphosphino)ferrocene]dichloropalladium (100 mg, 0.13 mmol) and triethylamine (1.0 mL) were added. The mixture was heated and reacted at 100° C. under microwave irradiation for 30 minutes, and then cooled to room temperature. At room temperature, the reaction mixture was diluted with water (30 mL). The resulting mixture was filtered. The filter cake was washed with ethyl acetate (10 mL). The filtrate was extracted with ethyl acetate (2×30 mL). The organic layers were combined, washed with brine (50 mL), dried over anhydrous Na₂SO₄, and filtered. The filtrate was concentrated under reduced pressure. The residue was purified with silica gel column (petroleum ether/ethyl acetate=1/0→1/1) to produce 5-((5-(4-cyclohexyl-5,6-dihydrocyclopenta[d][1,2,3]triazol-2 (4H)-yl)pyridin-3-yl)ethynyl)pyrimidin-2-amine (100 mg) as a white solid in a yield of 30.0%. 5 LC-MS (m/z): 386.5 [M+H]⁺. ¹H NMR (400 MHz, chloroform-d) δ 9.23 (s, 1H), 8.61 (s, 1H), 8.49 (s, 2H), 8.38 (t, J=2.0 Hz, 1H), 5.37 (brs, 2H), 3.02-2.92 (m, 1H), 2.90-2.73 (m, 2H), 2.72-2.62 (m, 1H), 2.38-2.25 (m, 1H), 2.17-2.05 (m, 2H), 1.81-1.74 (m, 3H), 1.72-1.64 (m, 1H), 1.55-1.44 (m, 1H), 1.32-1.12 (m, 3H), 1.12-1.00 (m, 1H).

The following intermediates were synthesized with reference to the synthesis of 6-cyclohexyl-1,4,5,6-tetrahydrocyclopenta[d][1,2,3]triazole.

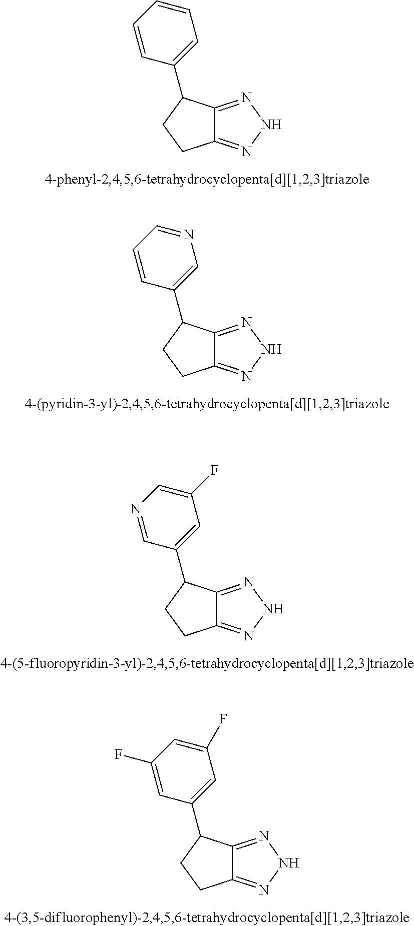 [SMILES: c1ccc(C2CCc3n[nH]nc32)cc1 ; c1cncc(C2CCc3n[nH]nc32)c1 ; Fc1cncc(C2CCc3n[nH]nc32)c1 ; Fc1cc(F)cc(C2CCc3n[nH]nc32)c1]

### Example 3: Synthesis of 5-((3-(6-phenyl-5,6-dihydrocyclopenta[c]pyrazol-2 (4H)-yl)cyclopentyl)ethynyl)pyrimidin-2-amine

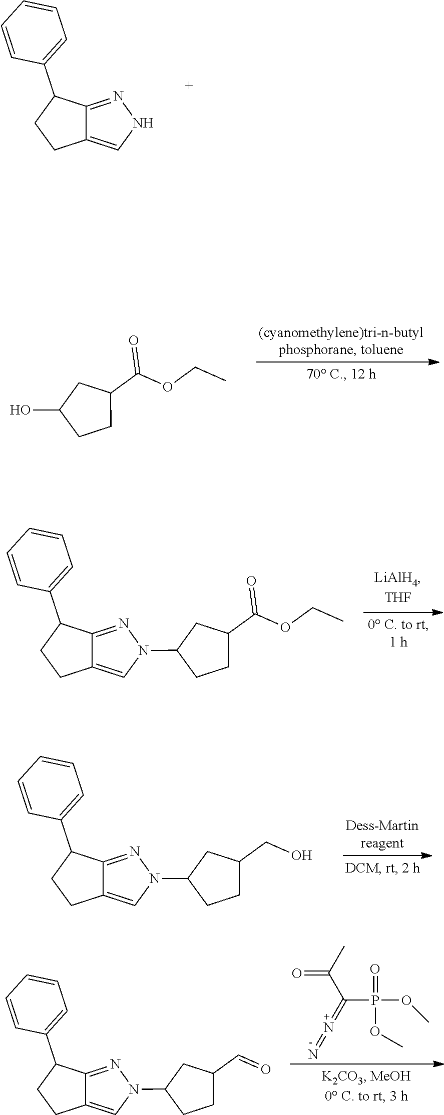

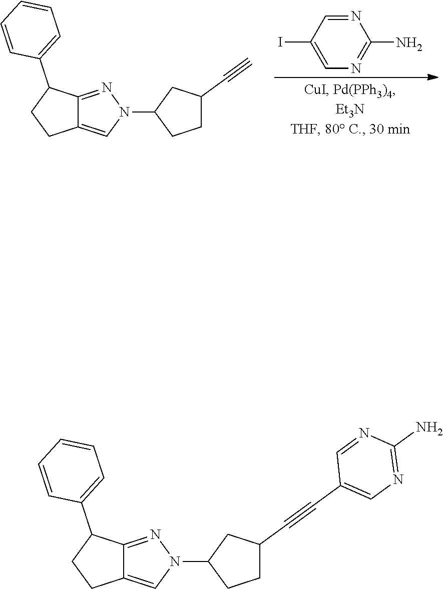

### Step 1: Synthesis of ethyl 3-(6-phenyl-5,6-dihydrocyclopenta[c]pyrazol-2 (4H)-yl)cyclopentane-1-carboxylate

6-phenyl-2,4,5,6-tetrahydrocyclopenta[c]pyrazole (2.5 g, 13.57 mmol) was dissolved in toluene (25 mL). The atmosphere was replaced with nitrogen gas three times. Ethyl 3-hydroxycyclopentane-1-carboxylate (2.15 g, 13.57 mmol) was added. The resulting mixture was stirred at room temperature for 5 minutes, and then (cyanomethylene)tri-n-butylphosphorane (3.93 g, 16.28 mmol) was added. The mixture was stirred at 70° C. overnight. LCMS detection indicated the reaction product formation. Ethyl acetate (100 mL) was added to dilute the reaction system. The resulting mixture was extracted with water (50 mL×3) and saturated brine (50 mL) in turn, and dried over anhydrous Na₂SO₄. The solvent was removed by rotary evaporation to dryness with a water pump. The resulting crude product was loaded to silica gel column and purified with silica gel column chromatography (petroleum ether:ethyl acetate=10:1 to 2:1) to produce ethyl 3-(6-phenyl-5,6-dihydrocyclopenta[c]pyrazol-2 (4H)-yl)cyclopentane-1-carboxylate (1.1 g) as a colourless oily substance in a yield of 25%. LC-MS (m/z): 325.1[M+H]⁺.

### Step 2: Synthesis of (3-(6-phenyl-5,6-dihydrocyclopenta[c]pyrazol-2 (4H)-yl)cyclopentyl) methanol

Ethyl 3-(6-phenyl-5,6-dihydrocyclopenta[c]pyrazol-2 (4H)-yl)cyclopentane-1-carboxylate (200 mg, 616.48 μmol) was dissolved in tetrahydrofuran (25 mL). Lithium aluminium hydride (31 mg, 0.80 mmol) was slowly added at 0° C. under nitrogen protection. The resulting mixture was naturally warmed up to room temperature and stirred for 1 hour. Then LCMS detection indicated the reaction product formation. The reaction was quenched by adding water (0.1 mL) at 0° C. Ethyl acetate (50 mL) was added to dilute the reaction system. The resulting mixture was extracted with water (25 mL×3) and saturated brine (25 mL) in turn, and dried over anhydrous Na₂SO₄. Then the solvent was removed by rotary evaporation to dryness with a water pump. The resulting crude product was loaded to silica gel column and purified with silica gel column chromatography (petroleum ether:ethyl acetate=8:1 to 1:1) to produce 3-(6-phenyl-5,6-dihydrocyclopenta[c]pyrazol-2 (4H)-yl)cyclopentyl) methanol (110 mg) as a colourless oily substance in a yield of 63%. LC-MS (m/z): 283.1[M+H]⁺.

### Step 3: Synthesis of 3-(6-phenyl-5,6-dihydrocyclopenta[c]pyrazol-2 (4H)-yl)cyclopentane-1-carbaldehyde

(3-(6-phenyl-5,6-dihydrocyclopenta[c]pyrazol-2 (4H)-yl)cyclopentyl)methanol (100 mg, 354.1 μmol) was dissolved in dichloromethane (5 mL). Then Dess-Martin reagent (300 mg, 708.3 μmol) was added. The mixture was stirred at room temperature for 2 hours. LCMS detection indicated the reaction product formation. Ethyl acetate (50 mL) was added to dilute the reaction system. The resulting mixture was extracted with water (25 mL×3) and saturated brine (25 mL) in turn, and dried over anhydrous Na₂SO₄. Then the solvent was removed by rotary evaporation to dryness with a water pump. The resulting crude product was loaded to silica gel column and purified with silica gel column chromatography (petroleum ether:ethyl acetate=20:1 to 1:1) to produce 3-(6-phenyl-5,6-dihydrocyclopenta[c]pyrazol-2 (4H)-yl)cyclopentane-1-carbaldehyde (80 mg) as a colourless oily substance in a yield of 80%. LC-MS (m/z): 281.1[M+H]⁺.

### Step 4: Synthesis of 2-(3-ethynylcyclopentyl)-6-phenyl-2,4,5,6-tetrahydrocyclopenta[c]pyrazole

3-(6-phenyl-5,6-dihydrocyclopenta[c]pyrazol-2 (4H)-yl)cyclopentane-1-carbaldehyde (80 mg, 285.3 μmol) was dissolved in methanol (5 mL). Potassium carbonate (197 mg, 1.43 mmol) was added. Then the atmosphere was replaced with nitrogen gas three times. Dimethyl (1-diazo-2-oxopropyl)phosphonate (1 g, in a 10% methanol solution, 535.01 μmol) was slowly added in an ice bath. The mixture was reacted under stirring at room temperature for 3 hours. LCMS detection indicated the reaction product formation. Ethyl acetate (50 mL) was added to dilute the reaction system. The resulting mixture was extracted with water (25 mL×3) and saturated brine (25 mL) in turn, and dried over anhydrous Na₂SO₄. Then the solvent was removed by rotary evaporation to dryness with a water pump. The resulting crude product was loaded to silica gel column and purified with silica gel column chromatography (petroleum ether:ethyl acetate=40:1 to 2:1) to produce 2-(3-ethynylcyclopentyl)-6-phenyl-2,4,5,6-tetrahydrocyclopenta[c]pyrazole (50 mg) as a colourless oily substance in a yield of 63.4%. LC-MS (m/z): 277.1 [M+H]⁺.

### Step 5: Synthesis of 5-((3-(6-phenyl-5,6-dihydrocyclopenta[c]pyrazol-2 (4H)-yl)cyclopentyl)ethynyl)pyrimidin-2-amine

2-(3-ethynylcyclopentyl)-6-phenyl-2,4,5,6-tetrahydrocyclopenta[c]pyrazole (50 mg, 180.9 μmol) was dissolved in tetrahydrofuran (3 mL). Then 5-iodopyrimidin-2-amine (48 mg, 217.09 μmol), cuprous iodide (4 mg, 18.09 μmol), tetrakis(triphenylphosphine)palladium (21 mg, 18.09 μmol) and triethylamine (184 mg, 1.81 mmol) were successively added. The atmosphere was replaced with nitrogen gas three times. The mixture was reacted at 80° C. in a microwave reactor for 0.5 hours. LCMS detection indicated the reaction product formation. Ethyl acetate (50 mL) was added to dilute the reaction system. The resulting mixture was extracted with water (25 mL×3) and saturated brine (25 mL) in turn, and dried over anhydrous Na₂SO₄. Then the solvent was removed by rotary evaporation to dryness with a water pump. The resulting crude product was purified with preparative HPLC to produce 5-((3-(6-phenyl-5,6-dihydrocyclopenta[c]pyrazol-2 (4H)-yl)cyclopentyl)ethynyl)pyrimidin-2-amine (22 mg) as a white solid in a yield of 33%. LC-MS (m/z): 370.2[M+H]⁺.

¹H NMR (400 MHz, DMSO) δ(ppm): δ 8.30 (s, 2H), 7.48 (d, J=1.2 Hz, 1H), 7.33-7.07 (m, 5H), 4.90-4.75 (m, 1H), 4.18 (dd, J=8.3, 6.7 Hz, 1H), 3.22 (pd, J=7.7, 4.9 Hz, 1H), 2.84 (dtd, J=12.7, 8.2, 4.4 Hz, 1H), 2.74-2.53 (m, 2H), 2.34-2.05 (m, 5H), 1.95 (tdd, J=10.4, 7.6, 5.6 Hz, 1H), 1.70 (dqd, J=12.2, 8.1, 2.2 Hz, 11H).

### Example 4: Synthesis of 4-((2-aminopyrimidin-5-yl)ethynyl)-1-methyl-6-(6-phenyl-5,6-dihydrocyclopenta[c]pyrazol-2 (4H)-yl)pyridine-2 (1H)-one

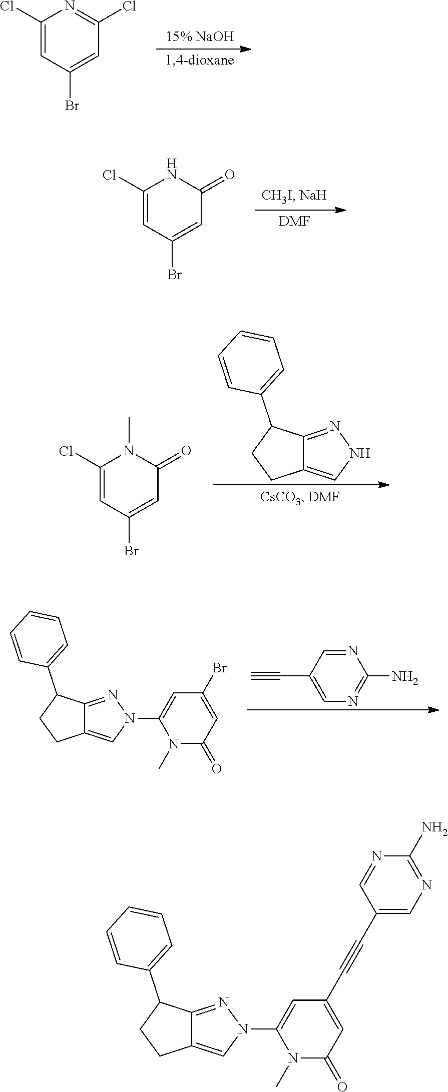

### Step 1: Synthesis of 4-bromo-6-chloropyridine-2 (1H)-one

4-bromo-2,6-dichloropyridine (2 g, 8.81 mmol) was dissolved in dioxane (8 mL), and 15% sodium hydroxide solution (8 mL) was added. The mixture was reacted under microwave at 150° C. for 30 minutes. After the completion of the reaction, the reaction system was cooled to room temperature, adjusted with 1N HCl to pH=6, and extracted with ethyl acetate (3×60 mL). The organic phases were combined, washed with brine, dried over anhydrous Na₂SO₄, filtered, and concentrated to produce 4-bromo-6-chloropyridine-2 (1H)-one (916 mg) as a white solid in a yield of 49.9%. LC-MS (m/z): 208.4, 210.3 [M]⁺.

### Step 2: Synthesis of 4-bromo-6-chloro-1-methylpyridin-2 (1H)-one

4-bromo-6-chloropyridine-2 (1H)-one (916 mg, 4.39 mmol) was dissolved in N,N-dimethylformamide (3 mL). To the resulting solution was added NaH (264 mg, 6.59 mmol) at 0° C. The mixture was reacted under nitrogen protection at 0° C. for 1 hour. Iodomethane (1.25 g, 8.79 mmol) was slowly added dropwise to the reaction mixture. The resulting reaction mixture was stirred at room temperature for 1 hour. The reaction mixture was poured into ice-water (10 mL), and extracted with ethyl acetate (3×30 mL). The organic phases were combined, washed with brine, dried over anhydrous Na₂SO₄, filtered, and rotary evaporated to dryness. The residue was purified with silica gel column (petroleum ether/ethyl acetate=1/1) to produce 4-bromo-6-chloro-1-methylpyridin-2 (1H)-one (443 mg) as a white solid in a yield of 45.4%. LC-MS (m/z): 222.4, 224.3 [M]⁺.

### Step 3: Synthesis of 4-bromo-1-methyl-6-(6-phenyl-5,6-dihydrocyclopenta[c]pyrazol-2 (4H)-yl)pyridine-2 (1H)-one

6-phenyl-2,4,5,6-tetrahydrocyclopenta[c]pyrazole hydrochloride (527 mg, 2.39 mmol), 4-bromo-6-chloro-1-methylpyridin-2 (1H)-one (443 mg, 1.99 mmol) and cesium carbonate (1.94 g, 5.97 mmol) were mixed into N,N-dimethylformamide (5 mL), and the mixture was reacted at 85° C. for 16 hours. Water (10 mL) was added to the reaction mixture. The resulting reaction mixture was extracted with ethyl acetate (3×50 mL). The organic phases were combined, washed with brine, dried over anhydrous Na₂SO₄, filtered, and rotary evaporated to dryness. The residue was purified with silica gel column (petroleum ether/ethyl acetate=2/1) to produce 4-bromo-1-methyl-6-(6-phenyl-5,6-dihydrocyclopenta[c]pyrazol-2 (4H)-yl)pyridine-2 (1H)-one (500 mg) as a yellow solid in a yield of 67.9%. LC-MS (m/z): 370.4, 372.3 [M]⁺.

### Step 4: Synthesis of 4-((2-aminopyrimidin-5-yl)ethynyl)-1-methyl-6-(6-phenyl-5,6-dihydrocyclopenta[c]pyrazol-2 (4H)-yl)pyridine-2 (1H)-one

Example 4 was synthesized with reference to the synthesis of Example 1 to produce an off-white solid (150 mg) in a yield of 68.2%.

LC-MS (m/z): 409.3[M+H]⁺. ¹H NMR (400 MHz, CDCl₃) δ 8.23 (dt, J=6.6, 2.6 Hz, 2H), 7.21-6.99 (m, 6H), 6.53 (dd, J=6.6, 3.6 Hz, 1H), 6.07 (q, J=5.1, 3.7 Hz, 1H), 5.20 (d, J=5.7 Hz, 2H), 4.25-4.07 (m, 1H), 3.16 (dt, J=6.5, 2.6 Hz, 3H), 2.78 (td, J=8.8, 4.4 Hz, 1H), 2.74-2.52 (m, 2H), 2.39-2.17 (m, 1H).

### Example 5: Synthesis of 3-((3-(6-phenyl-5,6-dihydrocyclopenta[c]pyrazol-2 (4H-ylphenylethynyl)imidazo[1,2-b]pyridazine

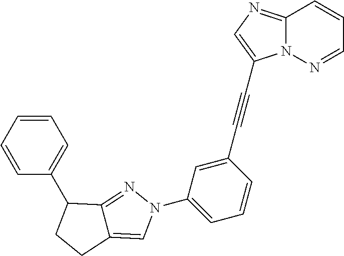 [SMILES: C(#Cc1cnc2cccnn12)c1cccc(-n2cc3c(n2)C(c2ccccc2)CC3)c1]

Example 5 was synthesized with reference to the synthesis of Example 1 to produce a white solid (23 mg) in a yield of 21.0%. LC-MS (m/z): 402.4 [M+H]⁺. ¹H NMR (400 MHz, chloroform-d) δ 8.54 (d, J=4.4 Hz, 1H), 8.17-8.09 (m, 2H), 7.94 (s, 1H), 7.70-7.64 (m, 2H), 7.50-7.37 (m, 2H), 7.32 (d, J=4.4 Hz, 4H), 7.24-7.17 (m, 2H), 4.38 (t, J=7.6 Hz, 1H), 3.04-2.92 (m, 1H), 2.90-2.71 (m, 2H), 2.48-2.38 (m, 11H).

### Example 6: Synthesis of 3-((5-(6-phenyl-5,6-dihydrocyclopenta[c]pyrazol-2 (4H)-yl)pyridin-3-yl)ethynyl)imidazo[1,2-b]pyridazine

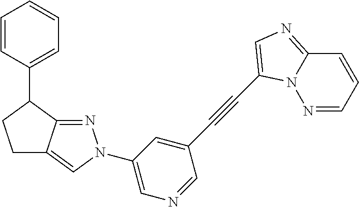 [SMILES: C(#Cc1cnc2cccnn12)c1cncc(-n2cc3c(n2)C(c2ccccc2)CC3)c1]

Example 6 was synthesized with reference to the synthesis of Example 1 to produce a white solid (11 mg) in a yield of 18.6%. LC-MS (m/z): 403.4 [M+H]⁺. ¹H NMR (400 MHz, DMSO-d₆) δ 9.09 (s, 1H), 8.73 (dd, J=4.4, 1.6 Hz, 1H), 8.64 (s, 1H), 8.41 (s, 1H), 8.35 (t, J=2.0 Hz, 1H), 8.32-8.23 (m, 2H), 7.42 (dd, J=9.2, 4.4 Hz, 1H), 7.37-7.28 (m, 4H), 7.27-7.18 (m, 1H), 4.35 (t, J=7.6 Hz, 1H), 2.93 (dtd, J=12.4, 8.0, 4.0 Hz, 1H), 2.87-2.65 (m, 2H), 2.36-2.27 (m, 1H).

### Example 7: Synthesis of 3-((2-(6-phenyl-5,6-dihydrocyclopenta[c]pyrazol-2 (4H)-yl)pyrimidine-4-yl)ethynyl)imidazo[1,2-b]pyridazine

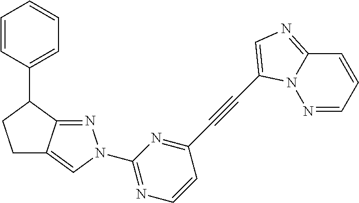 [SMILES: C(#Cc1cnc2cccnn12)c1ccnc(-n2cc3c(n2)C(c2ccccc2)CC3)n1]

Example 7 was synthesized with reference to the synthesis of Example 1 to produce a white solid (2 mg) in a yield of 1.2%. LC-MS (m/z): 404.4 [M+H]⁺. ¹H NMR (400 MHz, DMSO-d₆) δ 8.84 (d, J=5.2 Hz, 1H), 8.77 (dd, J=4.4, 1.6 Hz, 11H), 8.41 (s, 11H), 8.38 (s, 11H), 8.32 (dd, J=9.2, 1.6 Hz, 11H), 7.57 (d, J=5.2 Hz, 1H), 7.47 (dd, J=9.2, 4.4 Hz, 1H), 7.39-7.29 (m, 4H), 7.29-7.20 (m, 1H), 4.35 (t, J=7.8 Hz, 1H), 2.98-2.88 (m, 1H), 2.88-2.65 (m, 2H), 2.41-2.30 (m, 1H).

### Example 8: Synthesis of 3-((6-(6-phenyl-5,6-dihydrocyclopenta[c]pyrazol-2 (4H)-yl)pyrimidine-4-yl)ethynyl)imidazo[1,2-b]pyridazine

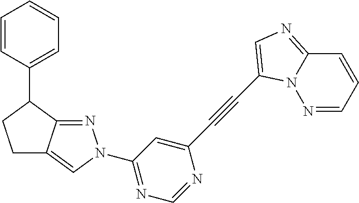 [SMILES: C(#Cc1cnc2cccnn12)c1cc(-n2cc3c(n2)C(c2ccccc2)CC3)ncn1]

Example 8 was synthesized with reference to the synthesis of Example 1 to produce a white solid (3 mg) in a yield of 2.0%. LC-MS (m/z): 404.4 [M+H]⁺. ¹H NMR (400 MHz, DMSO-d₆) δ 9.02 (d, J=1.2 Hz, 1H), 8.76 (dd, J=4.4, 1.6 Hz, 1H), 8.41 (d, J=1.2 Hz, 1H), 8.38 (s, 1H), 8.30 (dd, J=9.2, 1.6 Hz, 1H), 7.84 (d, J=1.2 Hz, 1H), 7.45 (dd, J=9.2, 4.4 Hz, 1H), 7.38-7.29 (m, 4H), 7.29-7.22 (m, 1H), 4.39 (t, J=8.0 Hz, 1H), 2.99-2.86 (m, 1H), 2.86-2.65 (m, 2H), 2.41-2.27 (m, 1H).

### Example 9: Synthesis of 5-((6-(6-phenyl-5,6-dihydrocyclopenta[c]pyrazol-2 (4H)-yl)pyrimidine-4-yl)ethynyl)pyrimidin-2-amine

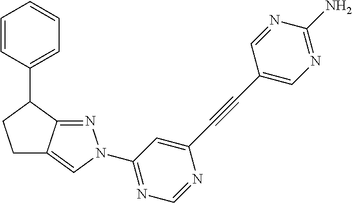 [SMILES: Nc1ncc(C#Cc2cc(-n3cc4c(n3)C(c3ccccc3)CC4)ncn2)cn1]

Example 9 was synthesized with reference to the synthesis of Example 1 to produce a yellow solid (8 mg) in a yield of 14.5%. LC-MS (m/z): 380.4 [M+H]⁺. ¹H NMR (400 MHz, DMSO-d₆) δ 8.98-8.89 (m, 1H), 8.58-8.50 (m, 2H), 8.38 (s, 1H), 7.85-7.76 (m, 1H), 7.69-7.47 (m, 1H), 7.44-7.37 (m, 1H), 7.37-7.19 (m, 5H), 4.43-4.30 (m, 1H), 2.99-2.61 (m, 3H), 2.39-2.26 (m, 1H).

### Example 10: Synthesis of 5-((5-(6-phenyl-5,6-dihydrocyclopenta[c]pyrazol-2 (4H)-yl)pyridin-3-yl)ethynyl)pyrimidine-2-carboxamide

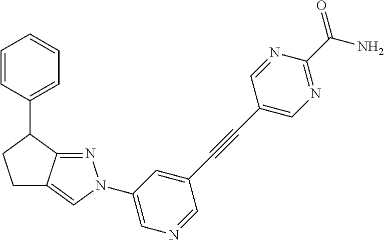 [SMILES: NC(=O)c1ncc(C#Cc2cncc(-n3cc4c(n3)C(c3ccccc3)CC4)c2)cn1]

Example 10 was synthesized with reference to the synthesis of Example 1 to produce a yellow solid (10.5 mg) in a yield of 8.4%. LC-MS (m/z): 407.4 [M+H]⁺. ¹H NMR (400 MHz, DMSO-d₆) δ 9.25-9.16 (m, 2H), 9.16-9.08 (m, 1H), 8.68 (s, 1H), 8.45-8.37 (m, 2H), 8.36-8.25 (m, 1H), 7.91 (s, 1H), 7.38-7.27 (m, 4H), 7.26-7.19 (m, 1H), 4.35 (t, J=7.6 Hz, 1H), 2.93 (ddt, J=12.4, 8.4, 4.0 Hz, 1H), 2.86-2.65 (m, 2H), 2.38-2.27 (m, 1H).

### Example 11: Synthesis of 6-((5-(6-phenyl-5,6-dihydrocyclopenta[c]pyrazol-2 (4H)-yl)pyridin-3-yl)ethynyl)pyridazine-3-ol

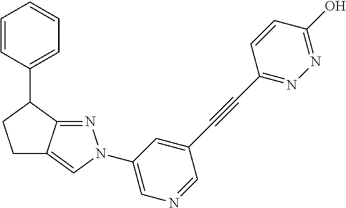 [SMILES: Oc1ccc(C#Cc2cncc(-n3cc4c(n3)C(c3ccccc3)CC4)c2)nn1]

Example 11 was synthesized with reference to the synthesis of Example 1 to produce a white solid (6.0 mg) in a yield of 5.3%. LC-MS (m/z): 380.4 [M+H]⁺. ¹H NMR (400 MHz, DMSO-d₆) δ 13.45 (brs, 1H), 9.09 (s, 1H), 8.63 (s, 1H), 8.37 (d, J=2.8 Hz, 2H), 7.66-7.63 (m, 1H), 7.36-7.28 (m, 4H), 7.26-7.20 (m, 11H), 6.97 (d, J=9.6 Hz, 11H), 4.35 (t, J=7.6 Hz, 11H), 2.93 (dtd, J=12.4, 8.0, 4.4 Hz, 1H), 2.86-2.65 (m, 2H), 2.36-2.29 (m, 1H).

### Example 12: Synthesis of 5-((5-(6-phenyl-5,6-dihydrocyclopenta[c]pyrazol-2 (4H)-yl)pyridin-3-yl)ethynyl)pyrazine-2-ol

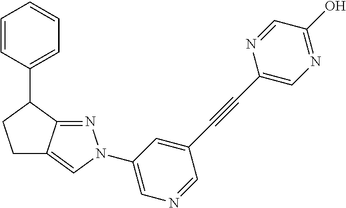 [SMILES: Oc1cnc(C#Cc2cncc(-n3cc4c(n3)C(c3ccccc3)CC4)c2)cn1]

Example 12 was synthesized with reference to the synthesis of Example 1 to produce a white solid (27 mg) in a yield of 24.1%. LC-MS (m/z): 380.4 [M+H]⁺. ¹H NMR (400 MHz, DMSO-d₆) δ 13.45 (brs ¹H NMR (400 MHz, DMSO-d₆) δ 8.91 (d, J=2.4 Hz, 1H), 8.45 (d, J=1.6 Hz, 1H), 8.35 (d, J=1.2 Hz, 1H), 8.15 (dd, J=2.4, 1.6 Hz, 1H), 7.93 (d, J=1.2 Hz, 1H), 7.36-7.28 (m, 4H), 7.25-7.19 (m, 2H), 4.35 (dd, J=8.4, 7.2 Hz, 1H), 2.99-2.86 (m, 1H), 2.83-2.65 (m, 2H), 2.38-2.25 (m, 1H).

### Example 13: Synthesis of (R)-5-((5-(6-phenyl-5,6-dihydrocyclopenta[c]pyrazol-2 (4H)-yl)pyridin-3-yl)ethynyl)pyrimidin-2-amine

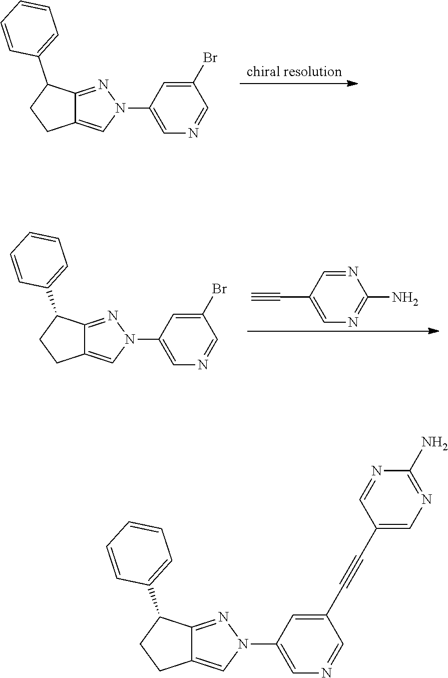

### Step 1: Synthesis of (R)-2-(5-bromopyridin-3-yl)-6-phenyl-2,4,5,6-tetrahydrocyclopenta[c]pyrazole

2-(5-bromopyridin-3-yl)-6-phenyl-2,4,5,6-tetrahydrocyclopenta[c]pyrazole was chirally resolved to produce (R)-2-(5-bromopyridin-3-yl)-6-phenyl-2,4,5,6-tetrahydrocyclopenta[c]pyrazole (2.23 g) as a white solid in a yield of 45.4%, ee value: 97.08%. LC-MS (m/z): 340.4 [M+H]⁺.

### Step 2: Synthesis of (R)-5-((5-(6-phenyl-5,6-dihydrocyclopenta[c]pyrazol-2 (4H)-yl)pyridin-3-yl)ethynyl)pyrimidin-2-amine

Example 13 was synthesized with reference to the synthesis of Example 1 to produce a white solid (75.0 mg) in a yield of 22.4%. LC-MS (m/z): 379.4 [M+H]⁺. ¹H NMR (400 MHz, DMSO-d₆) δ 9.01 (d, J=2.4 Hz, 1H), 8.54 (d, J=1.6 Hz, 1H), 8.48 (s, 2H), 8.36 (s, 1H), 8.26 (t, J=2.4 Hz, 1H), 7.37-7.28 (m, 4H), 7.27-7.21 (m, 3H), 4.34 (t, J=7.6 Hz, 1H), 2.98-2.88 (m, 1H), 2.87-2.67 (m, 2H), 2.37-2.27 (m, 1H).

### Example 14: Synthesis of (S)-5-(5-(6-phenyl-5,6-dihydrocyclopenta[c]pyrazol-2 (4H)-yl)pyridin-3-yl)ethynyl)pyrimidin-2-amine

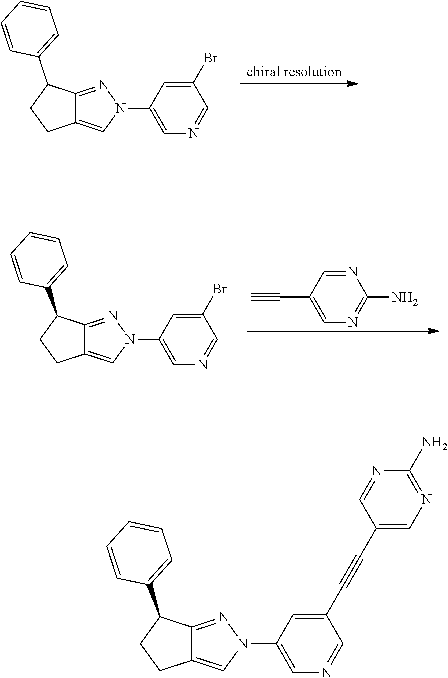

### Step 1: Synthesis of (S)-2-(5-bromopyridin-3-yl)-6-phenyl-2,4,5,6-tetrahydrocyclopenta[c]pyrazole

2-(5-bromopyridin-3-yl)-6-phenyl-2,4,5,6-tetrahydrocyclopenta[c]pyrazole was chirally resolved to produce (S)-2-(5-bromopyridin-3-yl)-6-phenyl-2,4,5,6-tetrahydrocyclopenta[c]pyrazole (2.03 g) as a white solid in a yield of 41.3%, ee value: 98.80%. LC-MS (m/z): 340.3 [M+H]⁺.

### Step 2: Synthesis of (S)-5-(5-(6-phenyl-5,6-dihydrocyclopenta[c]pyrazol-2 (4H)-yl)pyridin-3-yl)ethynyl)pyrimidin-2-amine

Example 14 was synthesized with reference to the synthesis of Example 1 to produce a grey solid (12 mg) in a yield of 5.4%. LC-MS (m/z): 379.4 [M+H]⁺. ¹H NMR (400 MHz, DMSO) δ 9.02 (d, J=2.5 Hz, 1H), 8.56-8.52 (m, 1H), 8.48 (s, 2H), 8.36 (s, 1H), 8.26 (t, J=2.2 Hz, 1H), 7.34-7.29 (m, 4H), 7.25 (s, 2H), 4.34 (t, J=7.7 Hz, 1H), 2.97-2.67 (m, 5H), 2.36-2.27 (m, 2H).

### Example 15: Synthesis of 3-((3-(6-phenyl-5,6-dihydrocyclopenta[c]pyrazol-2 (4H)-yl)phenyl)ethynyl)imidazo[1,2-a]pyridine

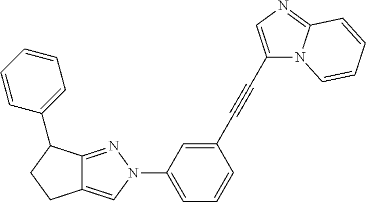 [SMILES: C(#Cc1cnc2ccccn12)c1cccc(-n2cc3c(n2)C(c2ccccc2)CC3)c1]

Example 15 was synthesized with reference to the synthesis of Example 1 to produce an off-white solid (25 mg) in a yield of 19.8%. LC-MS (m/z): 401.4 [M+H]⁺. ¹H NMR (400 MHz, chloroform-d) δ 8.44 (d, J=6.8 Hz, 1H), 7.96-7.91 (m, 2H), 7.90-7.80 (m, 1H), 7.71-7.60 (m, 2H), 7.47-7.40 (m, 2H), 7.38-7.35 (m, 1H), 7.34-7.29 (m, 4H), 7.25-7.19 (m, 1H), 7.10 (t, J=6.8 Hz, 1H), 4.37 (dt, J=8.4, 6.4 Hz, 1H), 3.03-2.91 (m, 1H), 2.90-2.71 (m, 2H), 2.50-2.36 (m, 1H).

### Example 16: Synthesis of 5-((5-(6-phenyl-5,6-dihydrocyclopenta[c]pyrazol-2 (4H)-yl)pyridin-3-yl)ethynyl)pyridine-2-amine

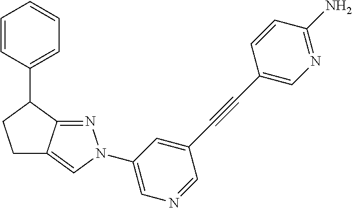 [SMILES: Nc1ccc(C#Cc2cncc(-n3cc4c(n3)C(c3ccccc3)CC4)c2)cn1]

Example 16 was synthesized with reference to the synthesis of Example 1 to produce a yellow solid (29.0 mg) in a yield of 26.1%. LC-MS (m/z): 378.4 [M+H]⁺. ¹H NMR (400 MHz, DMSO-d6) δ 8.98 (d, J=2.4 Hz, 1H), 8.52 (d, J=1.6 Hz, 1H), 8.36 (s, 1H), 8.23 (t, J=2.4 Hz, 1H), 8.17 (d, J=2.4 Hz, 1H), 7.55 (dd, J=8.4, 2.4 Hz, 1H), 7.37-7.26 (m, 4H), 7.23 (ddd, J=8.4, 5.6, 2.0 Hz, 1H), 6.52 (brs, 2H), 6.46 (d, J=8.4 Hz, 1H), 4.34 (t, J=7.6 Hz, 1H), 2.99-2.87 (m, 1H), 2.85-2.65 (m, 2H), 2.38-2.25 (m, 1H).

### Example 17: Synthesis of 6-((5-(6-phenyl-5,6-dihydrocyclopenta[c]pyrazol-2 (4H)-yl)pyridin-3-yl)ethynyl)pyridazine-3-amine

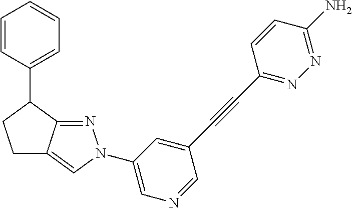 [SMILES: Nc1ccc(C#Cc2cncc(-n3cc4c(n3)C(c3ccccc3)CC4)c2)nn1]

Example 17 was synthesized with reference to the synthesis of Example 1 to produce a light-brown solid (38.0 mg) in a yield of 50.0%. LC-MS (m/z): 379.4 [M+H]⁺. ¹H NMR (400 MHz, DMSO-d₆) δ 9.06 (d, J=2.4 Hz, 1H), 8.61 (d, J=1.6 Hz, 1H), 8.38 (s, 1H), 8.36-8.27 (m, 1H), 7.50 (d, J=9.2 Hz, 1H), 7.37-7.28 (m, 4H), 7.27-7.18 (m, 1H), 6.90 (d, J=2.0 Hz, 2H), 6.78 (d, J=9.2 Hz, 1H), 4.35 (t, J=7.6 Hz, 1H), 2.98-2.88 (m, 1H), 2.87-2.65 (m, 2H), 2.37-2.27 (m, 1H).

### Example 18: Synthesis of 5-((5-(6-phenyl-5,6-dihydrocyclopenta[c]pyrazol-2 (4H)-yl)pyridin-3-yl)ethynyl)pyrazine-2-amine

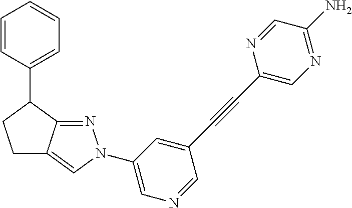 [SMILES: Nc1cnc(C#Cc2cncc(-n3cc4c(n3)C(c3ccccc3)CC4)c2)cn1]

Example 18 was synthesized with reference to the synthesis of Example 1 to produce a light-yellow solid (28.0 mg) in a yield of: 36.8%. LC-MS (m/z): 379.4 [M+H]⁺. ¹H NMR (400 MHz, chloroform-d) δ 8.94 (s, 1H), 8.60 (s, 1H), 8.34 (d, J=2.0 Hz, 1H), 8.21-8.16 (m, 1H), 8.09 (d, J=1.2 Hz, 1H), 7.67 (s, 1H), 7.37-7.30 (m, 4H), 7.25-7.22 (m, 1H), 5.52 (brs, 2H), 4.37 (t, J=7.6 Hz, 1H), 3.04-2.94 (m, 1H), 2.92-2.75 (m, 2H), 2.53-2.42 (m, 1H).

### Example 19: Synthesis of 5-((5-(6-(5-fluoropyridin-3-yl)-5,6-dihydrocyclopenta[c]pyrazol-2 (4H)-yl)pyridin-3-yl)ethynyl)pyrimidin-2-amine

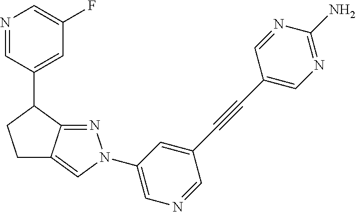 [SMILES: Nc1ncc(C#Cc2cncc(-n3cc4c(n3)C(c3cncc(F)c3)CC4)c2)cn1]

Example 19 was synthesized with reference to the synthesis of Example 1 to produce a white solid (6.0 mg) in a yield of 4.0%. LC-MS (m/z): 398.4 [M+H]⁺. ¹H NMR (400 MHz, DMSO-d₆) δ 9.02 (d, J=2.4 Hz, 1H), 8.56 (d, J=1.6 Hz, 1H), 8.48 (q, J=2.4 Hz, 4H), 8.39 (d, J=1.2 Hz, 1H), 8.27 (dd, J=2.4, 1.6 Hz, 1H), 7.65 (dt, J=10.0, 2.4 Hz, 1H), 7.25 (s, 2H), 4.50 (t, J=7.6 Hz, 1H), 2.98 (dtd, J=12.4, 8.0, 4.0 Hz, 1H), 2.91-2.72 (m, 2H), 2.44-2.34 (m, 1H).

### Example 20: Synthesis of 3-(2-(5-((2-aminopyrimidin-5-yl)ethynyl)pyridin-3-yl)-2,4,5,6-tetrahydrocyclopenta[c]pyrazol-6-yl)-5-fluorobenzonitrile

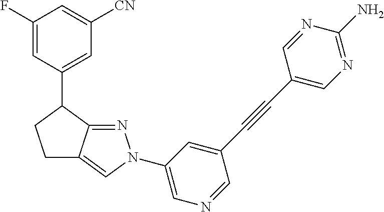 [SMILES: N#Cc1cc(F)cc(C2CCc3cn(-c4cncc(C#Cc5cnc(N)nc5)c4)nc32)c1]

Example 20 was synthesized with reference to the synthesis of Example 1 to produce a white solid (1.3 mg) in a yield of 1.2%. LC-MS (m/z): 422.4 [M+H]⁺. ¹H NMR (400 MHz, DMSO-d₆) δ 9.02 (d, J=2.4 Hz, 1H), 8.56 (d, J=1.6 Hz, 1H), 8.48 (s, 2H), 8.39 (d, J=1.2 Hz, 1H), 8.27 (dd, J=2.4, 1.6 Hz, 1H), 7.75 (ddd, J=8.4, 2.4, 1.2 Hz, 1H), 7.67 (t, J=1.6 Hz, 1H), 7.60-7.54 (m, 1H), 7.26 (s, 2H), 4.49 (t, J=7.6 Hz, 1H), 3.01-2.91 (m, 1H), 2.91-2.70 (m, 2H), 2.44-2.35 (m, 1H).

### Example 21: Synthesis of (S)-1-methyl-5-((5-(6-phenyl-5,6-dihydrocyclopenta[c]pyrazol-2 (4H)-yl)pyridin-3-yl)ethynyl)pyrimidine-2 (1H)-one

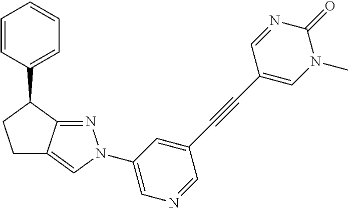 [SMILES: Cn1cc(C#Cc2cncc(-n3cc4c(n3)[C@H](c3ccccc3)CC4)c2)cnc1=O]

Example 21 was synthesized with reference to the synthesis of Example 1 to produce a white solid (15.0 mg) in a yield of 13.0%. LC-MS (m/z): 394.4 [M+H]⁺. ¹H NMR (400 MHz, DMSO-d₆) δ 9.04 (d, J=2.4 Hz, 1H), 8.74 (d, J=3.2 Hz, 1H), 8.70 (d, J=3.2 Hz, 1H), 8.54 (d, J=1.6 Hz, 1H), 8.37 (s, 1H), 8.29-8.24 (m, 1H), 7.37-7.27 (m, 4H), 7.27-7.19 (m, 1H), 4.34 (t, J=7.6 Hz, 1H), 3.46 (s, 3H), 2.97-2.88 (m, 1H), 2.85-2.69 (m, 2H), 2.41-2.33 (m, 1H).

### Example 22: Synthesis of 2-(3-((1,4-dimethyl-1H-pyrazole-5-yl)ethynyl)phenyl)-6-phenyl-2,4,5,6-tetrahydrocyclopenta[c]pyrazole

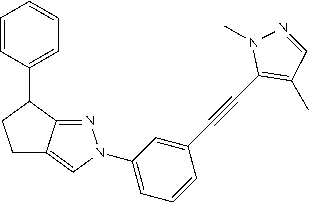 [SMILES: Cc1cnn(C)c1C#Cc1cccc(-n2cc3c(n2)C(c2ccccc2)CC3)c1]

Example 22 was synthesized with reference to the synthesis of Example 1 to produce a white solid (22 mg) in a yield of 33.1%. LC-MS (m/z): 379.4 [M+H]⁺. ¹H NMR (300 MHz, CDCl₃) δ 7.86 (t, J=1.5 Hz, 1H), 7.66-7.60 (m, 2H), 7.43-7.36 (m, 2H), 7.33-7.28 (m, 5H), 7.22 (ddd, J=8.6, 5.0, 3.7 Hz, 1H), 4.38 (dd, J=8.3, 6.6 Hz, 1H), 3.95 (s, 3H), 3.04-2.92 (m, 1H), 2.89-2.73 (m, 2H), 2.43 (ddt, J=12.7, 8.0, 6.5 Hz, 1H), 2.17 (s, 3H).

### Example 23: Synthesis of 2-(3-((1-methyl-1H-pyrazole-5-yl)ethynyl)phenyl)-6-phenyl-2,4,5,6-tetrahydrocyclopenta[c]pyrazole

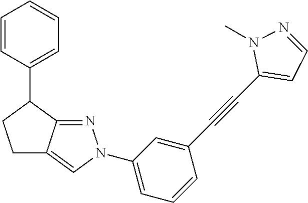 [SMILES: Cn1nccc1C#Cc1cccc(-n2cc3c(n2)C(c2ccccc2)CC3)c1]

Example 23 was synthesized with reference to the synthesis of Example 1 to produce a white solid (15 mg) in a yield of 20.8%. LC-MS (m/z): 465.4 [M+H]⁺. ¹H NMR (300 MHz, CDCl3) δ 8.38-8.03 (m, 1H), 7.86 (t, J=1.8 Hz, 1H), 7.78-7.28 (m, 8H), 7.22 (ddd, J=8.6, 4.9, 3.7 Hz, 1H), 6.51 (dd, J=12.2, 2.1 Hz, 1H), 4.37 (dd, J=8.2, 6.7 Hz, 1H), 4.06-3.95 (m, 3H), 3.06-2.67 (m, 3H), 2.43 (ddt, J=12.6, 8.0, 6.6 Hz, 1H).

### Example 24: Synthesis of 2-(3-((1,2-dimethyl-iH-imidazole-5-yl)ethynyl)phenyl)-6-phenyl-2,4,5,6-tetrahydrocyclopenta[c]pyrazole

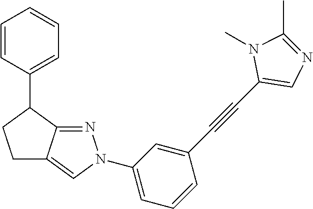 [SMILES: Cc1ncc(C#Cc2cccc(-n3cc4c(n3)C(c3ccccc3)CC4)c2)n1C]

Example 24 was synthesized with reference to the synthesis of Example 1 to produce a grey solid (7 mg) in a yield of 8.8%. LC-MS (m/z): 465.4 [M+H]⁺. ¹H NMR (300 MHz, CDCl3) δ 7.83 (s, 1H), 7.62 (d, J=9.2 Hz, 2H), 7.52-6.87 (m, 8H), 4.41-4.21 (m, 1H), 3.61 (s, 3H), 3.06-2.90 (m, 1H), 2.89-2.69 (m, 2H), 2.43 (dd, J=13.5, 7.2 Hz, 1H).

### Example 25: Synthesis of 2-(3-((1-methyl-iH-pyrazole-4-yl)ethynyl)phenyl)-6-phenyl-2,4,5,6-tetrahydrocyclopenta[c]pyrazole

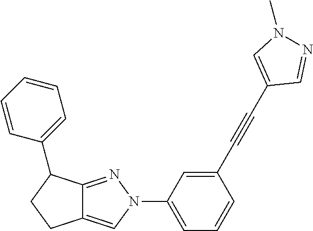 [SMILES: Cn1cc(C#Cc2cccc(-n3cc4c(n3)C(c3ccccc3)CC4)c2)cn1]

Example 25 was synthesized with reference to the synthesis of Example 1 to produce a yellow solid (18 mg) in a yield of 31.2%. LC-MS (m/z): 365.4 [M+H]⁺. ¹H NMR (400 MHz, chloroform-d) δ 7.79 (s, 1H), 7.62 (dd, J=18.3, 11.0 Hz, 4H), 7.32 (d, J=4.1 Hz, 5H), 7.21 (dt, J=8.7, 4.2 Hz, 1H), 4.37 (dd, J=8.3, 6.7 Hz, 1H), 3.92 (s, 3H), 3.02-2.71 (m, 4H), 2.42 (ddt, J=12.9, 8.3, 6.6 Hz, 1H).

### Example 26: Synthesis of 3-((6-(6-phenyl-5,6-dihydrocyclopenta[c]pyrazol-2 (4H)-yl)pyrazin-2-yl)ethynyl)imidazo[1,2-b]pyridazine

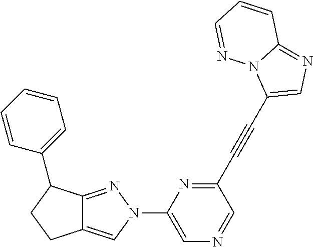 [SMILES: C(#Cc1cnc2cccnn12)c1cncc(-n2cc3c(n2)C(c2ccccc2)CC3)n1]

Example 26 was synthesized with reference to the synthesis of Example 1 to produce a light-yellow solid (18 mg) in a yield of 27.1%. LC-MS (m/z): 404.4 [M+H]⁺. ¹H NMR (400 MHz, chloroform-d) δ 9.18 (s, 1H), 8.64 (s, 1H), 8.54 (dd, J=4.5, 1.6 Hz, 1H), 8.28 (d, J=1.2 Hz, 1H), 8.19 (s, 1H), 8.06 (dd, J=9.2, 1.7 Hz, 1H), 7.34 (d, J=3.8 Hz, 4H), 7.21 (dt, J=9.2, 4.9 Hz, 1H), 4.38 (dd, J=8.3, 7.0 Hz, 1H), 2.98 (dtd, J=12.7, 8.2, 4.5 Hz, 1H), 2.92-2.73 (m, 2H), 2.46 (ddt, J=12.6, 8.3, 6.9 Hz, 1H).

### Example 27: Synthesis of 3-(2-(6-phenyl-5,6-dihydrocyclopenta[c]pyrazol-2 (4H)-yl)pyridine-4-yl)ethynyl)imidazo[1,2-b]pyridazine

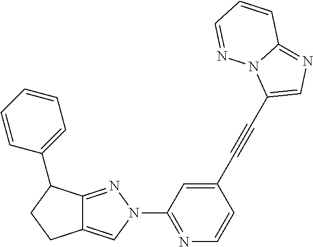 [SMILES: C(#Cc1cnc2cccnn12)c1ccnc(-n2cc3c(n2)C(c2ccccc2)CC3)c1]

Example 27 was synthesized with reference to the synthesis of Example 1 to produce a white solid (35 mg) in a yield of 56.1%. LC-MS (m/z): 403.4 [M+H]⁺. ¹H NMR (400 MHz, chloroform-d) δ 9.18 (s, 1H), 8.64 (s, 1H), 8.54 (dd, J=4.5, 1.6 Hz, 1H), 8.28 (d, J=1.2 Hz, 1H), 8.19 (s, 1H), 8.06 (dd, J=9.2, 1.7 Hz, 1H), 7.34 (d, J=3.8 Hz, 4H), 7.21 (dt, J=9.2, 4.9 Hz, 1H), 4.38 (dd, J=8.3, 7.0 Hz, 1H), 2.98 (dtd, J=12.7, 8.2, 4.5 Hz, 1H), 2.92-2.73 (m, 2H), 2.46 (ddt, J=12.6, 8.3, 6.9 Hz, 1H).

### Example 28: Synthesis of 5-(2-(6-phenyl-5,6-dihydrocyclopenta[c]pyrazol-2 (4H)-yl)pyridine-4-yl)ethynyl)pyrimidin-2-amine

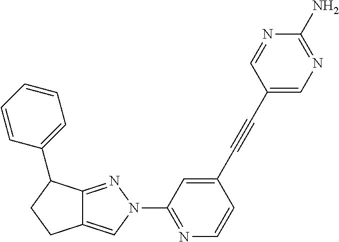 [SMILES: Nc1ncc(C#Cc2ccnc(-n3cc4c(n3)C(c3ccccc3)CC4)c2)cn1]

Example 28 was synthesized with reference to the synthesis of Example 1 to produce a white solid (12 mg) in a yield of 15.3%. LC-MS (m/z): 379.4 [M+H]⁺. ¹H NMR (400 MHz, chloroform-d) δ 8.56-8.15 (m, 4H), 8.00 (s, 1H), 7.51 (d, J=8.2 Hz, 1H), 7.41-7.05 (m, 5H), 5.32 (s, 2H), 4.35 (t, J=7.7 Hz, 1H), 2.66 (d, J=11.3 Hz, 1H), 2.51-2.28 (m, 2H), 2.16 (t, J=12.1 Hz, 1H).

### Example 29: Synthesis of 5-(6-(6-phenyl-5,6-dihydrocyclopenta[c]pyrazol-2 (4H)-yl)pyrazin-2-yl)ethynyl)pyrimidin-2-amine

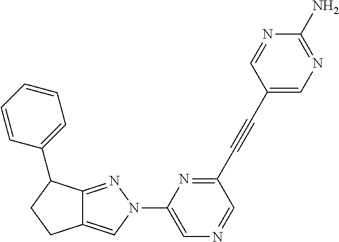 [SMILES: Nc1ncc(C#Cc2cncc(-n3cc4c(n3)C(c3ccccc3)CC4)n2)cn1]

Example 29 was synthesized with reference to the synthesis of Example 1 to produce a white solid (10 mg) in a yield of 11.2%. LC-MS (m/z): 380.4 [M+H]⁺. ¹H NMR (400 MHz, chloroform-d) δ 9.15 (d, J=0.5 Hz, 1H), 8.56 (s, 2H), 8.51 (d, J=0.6 Hz, 11H), 8.23 (d, J=1.2 Hz, 11H), 7.36-7.31 (m, 4H), 7.26 (s, 1H), 5.35 (s, 2H), 4.41-4.36 (m, 1H), 2.98 (dtd, J=12.8, 8.2, 4.5 Hz, 1H), 2.91-2.72 (m, 2H), 2.45 (ddt, J=12.7, 8.4, 6.9 Hz, 1H).

### Example 30: Synthesis of 6-phenyl-2-(5-(phenylethynyl)pyridin-3-yl)-2,4,5,6-tetrahydrocyclopenta[c]pyrazole

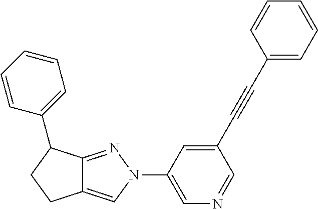 [SMILES: C(#Cc1cncc(-n2cc3c(n2)C(c2ccccc2)CC3)c1)c1ccccc1]

Example 30 was synthesized with reference to the synthesis of Example 1 to produce a white solid (21 mg) in a yield of 39.5%. LC-MS (m/z): 362.4 [M+H]⁺. ¹H NMR (400 MHz, chloroform-d) δ 8.77 (d, J=103.3 Hz, 1H), 8.17 (s, 1H), 7.66 (d, J=1.2 Hz, 1H), 7.57-7.50 (m, 2H), 7.41-7.29 (m, 7H), 7.22 (ddt, J=6.2, 4.7, 2.9 Hz, 2H), 4.37 (dd, J=8.3, 6.9 Hz, 1H), 2.97 (dtd, J=12.7, 8.3, 4.5 Hz, 1H), 2.92-2.73 (m, 2H), 2.44 (ddt, J=12.6, 8.3, 6.8 Hz, 1H).

### Example 31: Synthesis of (S)-5-(5-(6-phenyl-5,6-dihydrocyclopenta[c]pyrazol-2 (4H)-yl)pyridin-3-yl)ethynyl)thiazole-2-amine

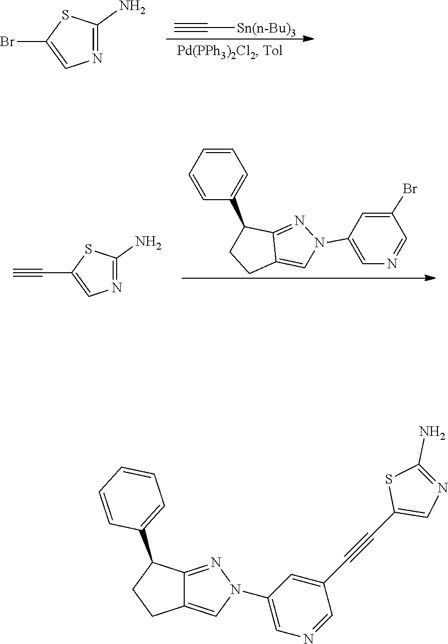

### Step 1: Synthesis of 5-ethynylthiazole-2-amine

Tributyl(ethynyl)stannane (2.1 g, 6.67 mmol) and bis(triphenylphosphine)palladium(II) dichloride (292 mg) were added to a suspension of 2-amino-5-bromothiazole (1.0 g, 5.585 mmol) in toluene (30 mL). The resulting mixture was stirred at 80° C. for 1 hour, then at 100° C. for 30 minutes, and at 120° C. for 30 minutes. The reaction mixture was concentrated. The residue was purified with silica gel column (ethyl acetate/petroleum ether=1/1) to produce 2-amino-5-ethynylthiazole (0.4 g) as a light-yellow solid in a yield of 57.5%. LC-MS (m/z): 125.4 [M+H]⁺.

### Step 2: Synthesis of (S)-5-(5-(6-phenyl-5,6-dihydrocyclopenta[c]pyrazol-2 (4H)-yl)pyridin-3-yl)ethynyl)thiazole-2-amine

Example 31 was synthesized with reference to the synthesis of Example 1 to produce an off-white solid (35 mg) in a yield of 62.1%. LC-MS (m/z): 384.4 [M+H]⁺. ¹H NMR (400 MHz, DMSO) δ 9.01 (s, 1H), 8.52 (s, 1H), 8.36 (s, 1H), 8.24 (d, J=2.0 Hz, 1H), 7.79 (s, 1H), 7.44 (s, 1H), 7.36-7.27 (m, 5H), 7.26-7.20 (m, 1H), 4.34 (dd, J=8.7, 6.6 Hz, 1H), 2.92-2.72 (m, 3H), 2.35-2.28 (m, 1H).

### Example 32: Synthesis of 6-phenyl-2-(3-(pyridin-3-ethynyl)phenyl)-2,4,5,6-tetrahydrocyclopenta[c]pyrazole

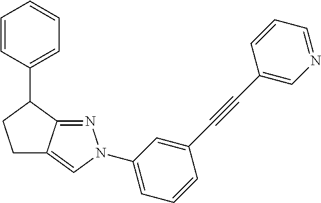 [SMILES: C(#Cc1cccc(-n2cc3c(n2)C(c2ccccc2)CC3)c1)c1cccnc1]

Example 32 was synthesized with reference to the synthesis of Example 1 to produce a white solid (32 mg) in a yield of 34.2%. LC-MS (m/z): 362.1 [M+H]⁺. ¹H NMR (400 MHz, chloroform-D) δ 8.69 (d, J=84.9 Hz, 2H), 7.90-7.85 (m, 1H), 7.81 (d, J=7.9 Hz, 1H), 7.64 (q, J=3.2, 2.3 Hz, 2H), 7.42-7.36 (m, 2H), 7.32 (d, J=4.3 Hz, 5H), 7.22 (h, J=4.2 Hz, 1H), 4.37 (dd, J=8.3, 6.7 Hz, 1H), 2.97 (dtd, J=12.8, 8.3, 4.6 Hz, 1H), 2.86 (ddd, J=15.3, 8.3, 4.5 Hz, 1H), 2.81-2.70 (m, 1H), 2.43 (ddt, J=13.0, 8.4, 6.7 Hz, 1H).

### Example 33: Synthesis of 2-(3-((6-methoxypyridin-3-yl)ethynyl)phenyl)-6-phenyl-2,4,5,6-tetrahydrocyclopenta[c]pyrazole

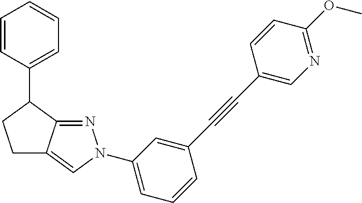 [SMILES: COc1ccc(C#Cc2cccc(-n3cc4c(n3)C(c3ccccc3)CC4)c2)cn1]

Example 33 was synthesized with reference to the synthesis of Example 1 to produce a white solid (38 mg) in a yield of 37.5%.

LC-MS (m/z): 392.2 [M+H]⁺. ¹H NMR (400 MHz, chloroform-D) δ 7.87 (t, J=1.8 Hz, 1H), 7.69-7.63 (m, 1H), 7.62 (s, 1H), 7.48 (d, J=8.6 Hz, 1H), 7.43-7.35 (m, 2H), 7.36-7.28 (m, 5H), 7.24-7.16 (m, 2H), 4.37 (dd, J=8.3, 6.8 Hz, 1H), 3.89 (s, 3H), 2.97 (dtd, J=12.7, 8.3, 4.5 Hz, 1H), 2.90-2.71 (m, 2H), 2.42 (ddt, J=12.9, 8.3, 6.7 Hz, 1H).

### Example 34: Synthesis of 2-(3-((5-methylpyridin-3-yl)ethynyl)phenyl)-6-phenyl-2,4,5,6-tetrahydrocyclopenta[c]pyrazole

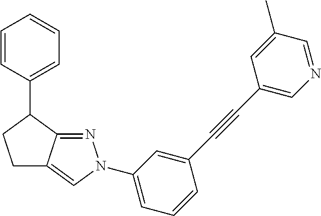 [SMILES: Cc1cncc(C#Cc2cccc(-n3cc4c(n3)C(c3ccccc3)CC4)c2)c1]

Example 34 was synthesized with reference to the synthesis of Example 1 to produce a white solid (12 mg) in a yield of 18.2%.

LC-MS (m/z): 376.2 [M+H]⁺. ¹H NMR (400 MHz, chloroform-D) δ 7.86 (dd, J=2.5, 1.3 Hz, 1H), 7.64 (ddt, J=5.9, 4.5, 2.3 Hz, 4H), 7.42-7.36 (m, 3H), 7.34-7.30 (m, 5H), 7.25-7.19 (m, 1H), 4.37 (dd, J=8.3, 6.7 Hz, 1H), 2.97 (dtd, J=12.8, 8.3, 4.6 Hz, 1H), 2.86 (dddd, J=15.2, 8.4, 4.6, 1.0 Hz, 1H), 2.81-2.72 (m, 1H), 2.47-2.40 (m, 1H), 2.38 (s, 3H).

### Example 35: Synthesis of 2-(3-((6-methylpyridin-3-yl)ethynyl)phenyl)-6-phenyl-2,4,5,6-tetrahydrocyclopenta[c]pyrazole

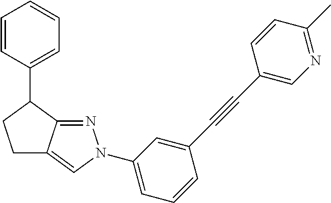 [SMILES: Cc1ccc(C#Cc2cccc(-n3cc4c(n3)C(c3ccccc3)CC4)c2)cn1]

Example 35 was synthesized with reference to the synthesis of Example 1 to produce a white solid (24 mg) in a yield of 36.4%. LC-MS (m/z): 376.2 [M+H]⁺. ¹H NMR (400 MHz, chloroform-D) δ 7.87-7.84 (m, 1H), 7.73-7.68 (m, 1H), 7.65-7.60 (m, 2H), 7.44-7.34 (m, 3H), 7.31 (d, J=4.3 Hz, 4H), 7.21 (dt, J=8.7, 4.4 Hz, 1H), 4.36 (dd, J=8.3, 6.7 Hz, 1H), 2.96 (dtd, J=12.8, 8.3, 4.7 Hz, 1H), 2.90-2.67 (m, 2H), 2.57 (s, 3H), 2.41 (ddt, J=12.9, 8.3, 6.6 Hz, 1H).

### Example 36: Synthesis of 5-((3-(6-phenyl-5,6-dihydrocyclopenta[c]pyrazol-2 (4H)-yl)phenyl)ethynyl)pyrimidin-2-amine

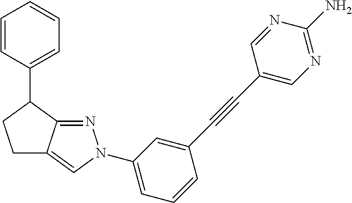 [SMILES: Nc1ncc(C#Cc2cccc(-n3cc4c(n3)C(c3ccccc3)CC4)c2)cn1]

Example 36 was synthesized with reference to the synthesis of Example 1 to produce a white solid (32 mg) in a yield of 48.4%. LC-MS (m/z): 378.2 [M+H]⁺. ¹H NMR (400 MHz, chloroform-D) δ 8.45 (s, 2H), 7.83 (t, J=1.8 Hz, 1H), 7.66-7.59 (m, 2H), 7.38 (t, J=7.8 Hz, 1H), 7.35-7.29 (m, 5H), 7.22 (p, J=4.4 Hz, 1H), 5.31 (s, 2H), 4.37 (dd, J=8.4, 6.7 Hz, 1H), 2.97 (dtd, J=12.8, 8.2, 4.6 Hz, 1H), 2.91-2.69 (m, 2H), 2.42 (ddt, J=13.0, 8.3, 6.6 Hz, 1H).

### Example 37: Synthesis of 3-(4-(6-phenyl-5,6-dihydrocyclopenta[c]pyrazol-2 (4H)-yl)pyridin-2-yl)ethynyl)imidazo[1,2-b]pyridazine

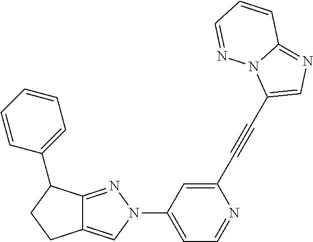 [SMILES: C(#Cc1cnc2cccnn12)c1cc(-n2cc3c(n2)C(c2ccccc2)CC3)ccn1]

Example 37 was synthesized with reference to the synthesis of Example 1 to produce a yellow solid (12 mg) in a yield of 21.4%. LC-MS (m/z): 403.2 [M+H]⁺. ¹H NMR (400 MHz, chloroform-d) δ 8.65-8.53 (m, 1H), 8.50 (d, J=4.4 Hz, 1H), 8.23-8.10 (m, 1H), 8.04 (d, J=9.2 Hz, 1H), 7.99-7.90 (m, 1H), 7.74 (s, 1H), 7.59-7.52 (m, 1H), 7.37-7.29 (m, 4H), 7.25-7.22 (m, 1H), 7.16 (dd, J=9.2, 4.4 Hz, 1H), 4.37 (t, J=7.6 Hz, 1H), 3.02-2.93 (m, 1H), 2.93-2.73 (m, 2H), 2.52-2.38 (m, 1H).

### Example 38: Synthesis of 5-(5-(6-phenyl-5,6-dihydrocyclopenta[c]pyrazol-2 (4H)-yl)pyridin-3-yl)ethynyl)pyrimidin-2-amine

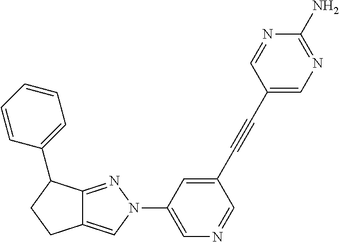 [SMILES: Nc1ncc(C#Cc2cncc(-n3cc4c(n3)C(c3ccccc3)CC4)c2)cn1]

Example 38 was synthesized with reference to the synthesis of Example 1 to produce an off-white solid (10.1 mg) in a yield of 9.1%. LC-MS (m/z): 379.2 [M+H]⁺. ¹H NMR (400 MHz, DMSO-D6) δ 8.98 (d, J=2.6 Hz, 1H), 8.50 (d, J=1.8 Hz, 1H), 8.44 (s, 2H), 8.33 (s, 1H), 8.22 (t, J=2.2 Hz, 1H), 7.28 (d, J=1.8 Hz, 3H), 7.21 (s, 2H), 4.31 (t, J=7.7 Hz, 1H), 2.94-2.85 (m, 1H), 2.70 (q, J=7.5 Hz, 1H), 2.62 (s, 1H), 2.28 (s, 1H).

### Example 39: 5-(5-(3-chloro-6-phenyl-5,6-dihydrocyclopenta[c]pyrazol-2 (4H)-yl)pyridin-3-yl)ethynyl)pyrimidin-2-amine

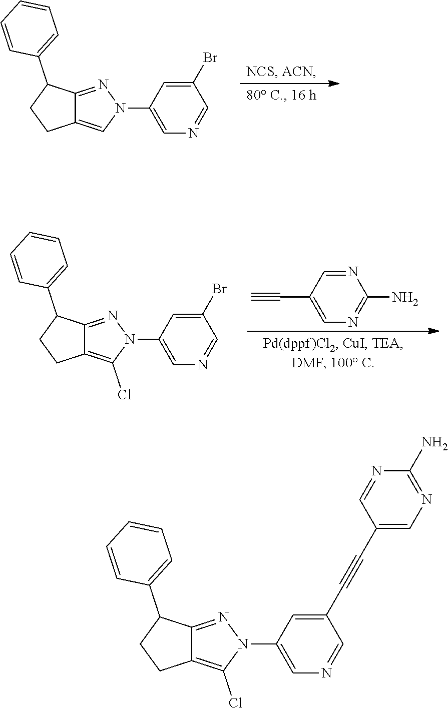

### Step 1: Synthesis of 2-(5-bromopyridin-3-yl)-3-chloro-6-phenyl-2,4,5,6-tetrahydrocyclopenta[c]pyrazole

2-(5-bromopyridin-3-yl)-6-phenyl-2,4,5,6-tetrahydrocyclopenta[c]pyrazole (500 mg, 1.47 mmol) was dissolved in acetonitrile (2 mL). N-chlorosuccinimide (393 mg, 3 mmol) was added at room temperature. The mixture was reacted at 80° C. overnight. TLC detection indicated the completion of the reaction of raw materials. The reaction mixture was rotary evaporated to dryness, and purified with silica gel column (petroleum ether/ethyl acetate=2/1) to produce 2-(5-bromopyridin-3-yl)-3-chloro-6-phenyl-2,4,5,6-tetrahydrocyclopenta[c]pyrazole (350 mg) as a white solid in a yield of 63.6%. LC-MS (m/z): 375.2 [M+H]⁺.

### Step 2: Synthesis of 5-(5-(3-chloro-6-phenyl-5,6-dihydrocyclopenta[c]pyrazol-2 (4H)-yl)pyridin-3-yl)ethynyl)pyrimidin-2-amine

Example 39 was synthesized with reference to the synthesis of Example 1 to produce a grey solid (11 mg) in a yield of 25.6%. LC-MS (m/z): 413.2 [M+H]⁺. ¹H NMR (400 MHz, DMSO) δ 8.79 (d, J=2.4 Hz, 1H), 8.74 (d, J=2.0 Hz, 1H), 8.48 (s, 2H), 8.14 (t, J=2.2 Hz, 1H), 7.33 (d, J=4.3 Hz, 3H), 7.28-7.20 (m, 2H), 4.39 (t, J=7.8 Hz, 1H), 2.98-2.88 (m, 1H), 2.87-2.69 (m, 2H), 2.40-2.32 (m, 1H).

### Example 40: Synthesis of 5-(5-(3-methyl-6-phenyl-5,6-dihydrocyclopenta[c]pyrazol-2 (4H)-yl)pyridin-3-yl)ethynyl)pyrimidin-2-amine

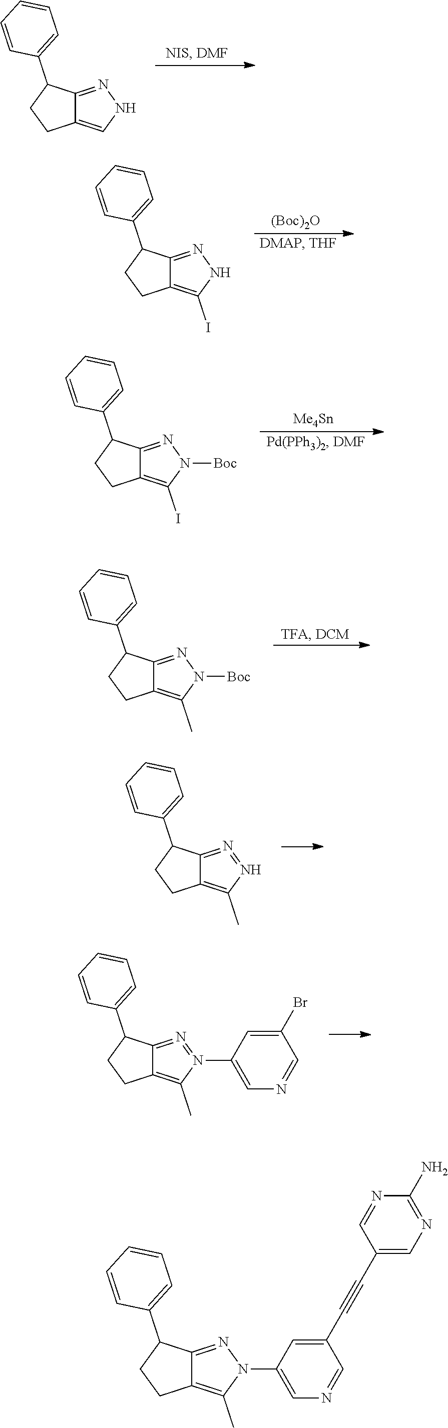

### Step 1: Synthesis of 3-iodo-6-phenyl-2,4,5,6-tetrahydrocyclopenta[c]pyrazole

6-phenyl-2,4,5,6-tetrahydrocyclopenta[c]pyrazole (3.0 g, 16.2 mmol) was dissolved in N,N-dimethylformamide (15 mL). N-iodosuccinimide (5.5 g, 24.4 mmol) was added in portions at room temperature. The mixture was reacted under nitrogen protection at 80° C. for 3 hours, diluted with water (15 mL), and extracted with ethyl acetate (3×60 mL). The organic phases were combined, washed with brine, and rotary-evaporated to dryness under reduced pressure. The residue was purified with silica gel column (petroleum ether/ethyl acetate=1/1) to produce 3-iodo-6-phenyl-2,4,5,6-tetrahydrocyclopenta[c]pyrazole (3.8 g) as a light-yellow solid in a yield of 75.5%. LC-MS (m/z): 311.2 [M+H]⁺.

### Step 2: Synthesis of tert-butyl 3-iodo-6-phenyl-5,6-dihydrocyclopenta[c]pyrazole-2 (4H)-carboxylate

3-iodo-6-phenyl-2,4,5,6-tetrahydrocyclopenta[c]pyrazole (2.3 g, 7.4 mmol) was dissolved in tetrahydrofuran (10 mL). 4-dimethylaminopyridine (230 mg, 1.88 mmol) and triethylamine (2.2 g, 22.2 mmol) were added. The resulting mixture was reacted at room temperature for 16 hours. The reaction mixture was rotary evaporated to dryness. The residue was purified with silica gel column (petroleum ether/ethyl acetate=3/1) to produce tert-butyl 3-iodo-6-phenyl-5,6-dihydrocyclopenta[c]pyrazole-2 (4H)-carboxylate (2 g) as a light-yellow solid in a yield of 66%. LC-MS (m/z): 411.2 [M+H]⁺.

### Step 3: Synthesis of tert-butyl 3-methyl-6-phenyl-5,6-dihydrocyclopenta[c]pyrazole-2 (4H)-carboxylate

Tert-butyl 3-iodo-6-phenyl-5,6-dihydrocyclopenta[c]pyrazole-2 (4H)-carboxylate (1.233 g, 3 mmol), tetramethylstannane (5.4 g, 30 mmol) and Pd(PPh₃)₂Cl₂ (210 mg, 0.3 mmol) were mixed into N,N-dimethylformamide (40 mL). The mixture was reacted at 100° C. for 16 hours, diluted with water (15 mL), and extracted with ethyl acetate (3×30 mL). The organic phases were combined, washed with brine, and rotary-evaporated to dryness under reduced pressure. The residue was purified with silica gel column (petroleum ether/ethyl acetate=1/2) to produce tert-butyl 3-methyl-6-phenyl-5,6-dihydrocyclopenta[c]pyrazole-2 (4H)-carboxylate (247 mg) as a white solid in a yield of 27.6%. LC-MS (m/z): 299.2 [M+H]⁺.

### Step 4: Synthesis of 3-methyl-6-phenyl-2,4,5,6-tetrahydrocyclopenta[c]pyrazole

tert-butyl 3-methyl-6-phenyl-5,6-dihydrocyclopenta[c]pyrazole-2 (4H)-carboxylate (247 mg, 083 mmol) was dissolved in dichloromethane (5 mL), and trifluoroacetic acid (0.5 mL) was added in an ice bath. The reaction mixture was stirred at room temperature for 1 hour, and rotary evaporated to dryness. The resulting crude was directly used in the next step. LC-MS (m/z): 199.2 [M+H]⁺.

### Step 5 and Step 6: Synthesis of 2-(5-bromopyridin-3-yl)-3-methyl-6-phenyl-2,4,5,6-tetrahydrocyclopenta[c]pyrazole and 5-(5-(3-methyl-6-phenyl-5,6-dihydrocyclopenta[c]pyrazol-2 (4H)-yl)pyridin-3-yl)ethynyl)pyrimidin-2-amine

Example 40 was synthesized with reference to the synthesis of Example 1 to produce a white solid (60 mg) in a yield of 27.0%. LC-MS (m/z): 393.0 [M+H]⁺. ¹H NMR (400 MHz, CDCl₃) δ 8.70 (s, 2H), 8.47 (s, 2H), 8.03 (s, 1H), 7.31 (d, J=4.4 Hz, 4H), 7.21 (dt, J=8.7, 4.3 Hz, 1H), 6.20 (s, 2H), 4.33 (t, J=7.6 Hz, 1H), 2.95 (dtd, J=12.6, 8.1, 4.3 Hz, 1H), 2.82-2.64 (m, 2H), 2.51-2.36 (m, 4H).

### Example 41: Synthesis of 2-(5-((2-chloropyrimidin-5-yl)ethynyl)pyridin-3-yl)-6-phenyl-2,4,5,6-tetrahydrocyclopenta[c]pyrazole

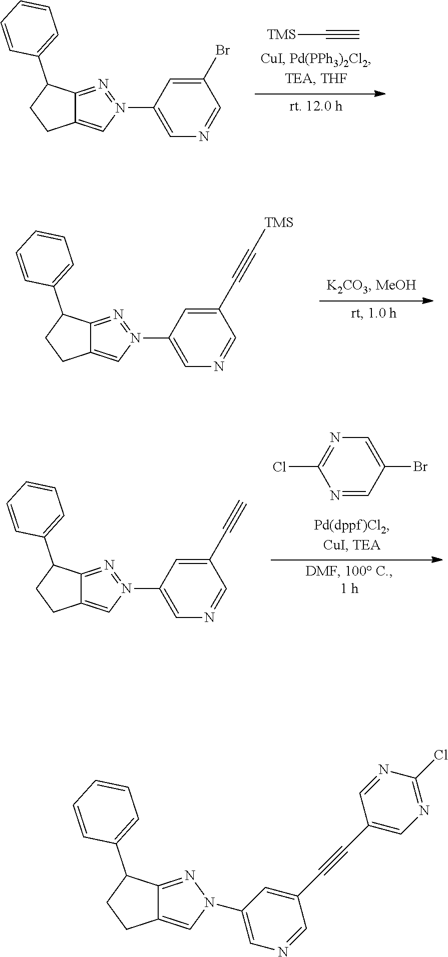

### Step 1: Synthesis of 6-phenyl-2-(5-((trimethylsilyl)ethynyl)pyridin-3-yl)-2,4,5,6-tetrahydrocyclopenta[c]pyrazole

2-(5-bromopyridin-3-yl)-6-phenyl-2,4,5,6-tetrahydrocyclopenta[c]pyrazole (95.3 mg, 0.28 mmol), ethynyltrimethylsilane(31 mg, 0.31 mmol), Pd(PPh₃)₂Cl₂ (20 mg, 0.06 mmol), cuprous iodide (5.4 mg, 0.01 mmol) and triethylamine (58 mg, 0.57 mmol) were mixed into tetrahydrofuran (5 mL). The reaction mixture was stirred under nitrogen protection at room temperature for 12.0 hours. After the completion of the reaction, the resulting reaction mixture was diluted with water (10 mL), extracted with ethyl acetate (3×15 mL), washed with brine, dried over anhydrous Na₂SO₄, filtered, and rotary evaporated to dryness to produce 6-phenyl-2-(5-((trimethylsilyl)ethynyl)pyridin-3-yl)-2,4,5,6-tetrahydrocyclopenta[c]pyrazole (86.5 mg) as a brown oil in a yield of 86.4%, LC-MS (m/z): 358.5 [M+H]⁺.

### Step 2: Synthesis of 2-(5-ethynylpyridin-3-yl)-6-phenyl-2,4,5,6-tetrahydrocyclopenta[c]pyrazole

6-phenyl-2-(5-((trimethylsilyl)ethynyl)pyridin-3-yl)-2,4,5,6-tetrahydrocyclopenta[c]pyrazole (86.5 mg, 0.242 mmol) was dissolved in methanol (5 mL). Potassium carbonate (11.5 mg, 0.30 mmol) was added. The resulting mixture was stirred at room temperature for 1.0 hour. TLC detection indicated the completion of the reaction. The reaction mixture was concentrated under reduced pressure, and purified with reverse C₁₈ column (35%-50% acetonitrile) to produce 2-(5-ethynylpyridin-3-yl)-6-phenyl-2,4,5,6-tetrahydrocyclopenta[c]pyrazole (45 mg) as a yellow-brown solid in a yield of 65.2%. LC-MS (m/z): 286.3 [M+H]⁺.

### Step 3: Synthesis of 2-(5-((2-chloropyrimidin-5-yl)ethynyl)pyridin-3-yl)-6-phenyl-2,4,5,6-tetrahydrocyclopenta[c]pyrazole

2-(5-ethynylpyridin-3-yl)-6-phenyl-2,4,5,6-tetrahydrocyclopenta[c]pyrazole (1.1 g, 3.50 mmol), 5-bromo-2-chloropyrimidine (825 mg, 3.80 mmol), cuprous iodide (105 mg, 0.35 mmol), Pd(dppf)₂Cl₂ (360 mg, 0.35 mmol) and triethylamine (1.59 g, 10.5 mmol) were mixed into N,N-dimethylformamide (10 mL). The reaction mixture was stirred under nitrogen protection at 100° C. for 1-2 hours. LCMS detection indicated the disappearance of raw materials. Water (5 mL) was added. The resulting mixture was extracted with ethyl acetate (3×50 mL), washed with brine, and dried over anhydrous Na₂SO₄. The organic phases were combined, and concentrated under reduced pressure. The residue was purified with preparative HPLC to produce 2-(5-((2-chloropyrimidin-5-yl)ethynyl)pyridin-3-yl)-6-phenyl-2,4,5,6-tetrahydrocyclopenta[c]pyrazole (400 mg) as a yellow solid in a yield of 29%. LC-MS (m/z): 398.2 [M+H]⁺. ¹H NMR (400 MHz, chloroform-d) δ8.94 (d, J=2.4 Hz, 1H), 8.77 (s, 2H), 8.62-8.57 (m, 1H), 8.56-8.46 (m, 1H), 8.44-8.35 (m, 1H), 7.70 (s, 1H), 7.38-7.28 (m, 4H), 4.37 (t, J=7.6 Hz, 1H), 3.05-2.75 (m, 3H), 2.53-2.40 (m, 1H).

### Example 42: Synthesis of 5-((6-methyl-5-(6-phenyl-5,6-dihydrocyclopenta[c]pyrazol-2 (4H)-yl)pyridin-3-yl)ethynyl)pyrimidin-2-amine

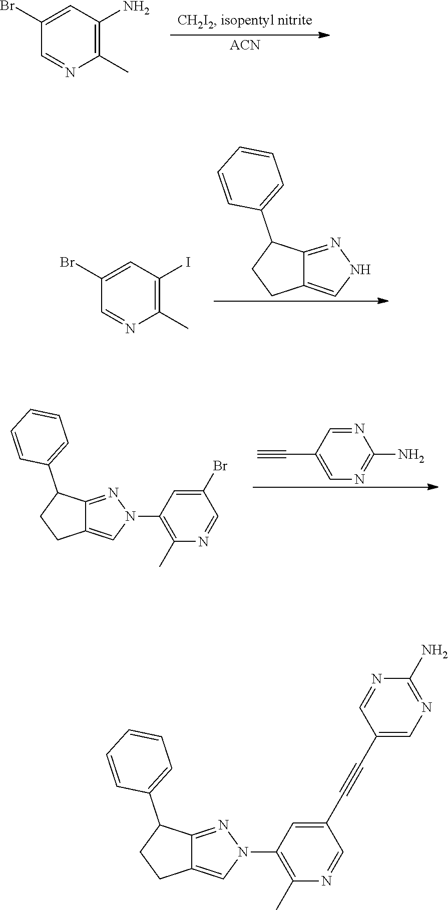

### Step 1: Synthesis of 5-bromo-3-iodo-2-methylpyridine

5-bromo-2-methylpyridine-3-amine (5.00 g, 26.73 mmol), and diiodomethane (35.8 g, 133.66 mmol) were added to acetonitrile (150 mL). At 0° C., under the protection of nitrogen gas, isopentyl nitrite (6.888 g, 58.8 mmol) was added. The mixture was reacted at room temperature for 16 hours. After the completion of the reaction, the reaction system was concentrated under reduced pressure. The residue was purified with a silic gel column (petroleum ether/ethyl acetate=2/1) to produce 5-bromo-3-iodo-2-methylpyridine (3.5 g) as a brown solid in a yield of 44%. LC-MS (m/z): 298.2 [M+H]⁺.

### Step 2 and Step 3: Synthesis of 2-(5-bromo-2-methylpyridin-3-yl)-6-phenyl-2,4,5,6-tetrahydrocyclopenta[c]pyrazole and 5-((6-methyl-5-(6-phenyl-5,6-dihydrocyclopenta[c]pyrazol-2 (4H)-yl)pyridin-3-yl)ethynyl)pyrimidin-2-amine

Example 42 was synthesized with reference to the synthesis of Example 1 to produce an off-white solid (158 mg) in a yield of 37.6%. LC-MS (m/z): 393.2 [M+H]⁺. ¹H NMR (400 MHz, CDCl3) δ 8.60 (d, J=2.1 Hz, 1H), 8.45 (s, 2H), 7.80 (d, J=2.1 Hz, 1H), 7.35-7.26 (m, 5H), 7.22 (dt, J=8.9, 4.4 Hz, 1H), 5.35 (s, 2H), 4.38 (t, J=7.7 Hz, 1H), 2.99 (dtd, J=12.6, 8.2, 4.3 Hz, 1H), 2.92-2.69 (m, 2H), 2.47 (dt, J=13.4, 7.4 Hz, 1H).

### Example 43: Synthesis of 5-((4-methyl-5-(6-phenyl-5,6-dihydrocyclopenta[c]pyrazol-2 (4H)-yl)pyridine-3-yl)ethynyl)pyrimidin-2-amine

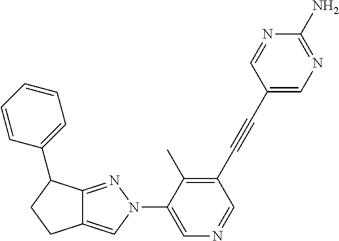 [SMILES: Cc1c(C#Cc2cnc(N)nc2)cncc1-n1cc2c(n1)C(c1ccccc1)CC2]

Example 43 was synthesized with reference to the synthesis of Example 1 to produce an off-white solid (63 mg) in a yield of 23%. LC-MS (m/z): 393.2 [M+H]⁺. ¹H NMR (400 MHz, chloroform-D) δ 8.64 (s, 1H), 8.48 (d, J=12.4 Hz, 3H), 7.37-7.28 (m, 5H), 7.20 (ddt, J=8.6, 5.6, 2.5 Hz, 1H), 5.27 (s, 2H), 4.38 (dd, J=8.3, 7.1 Hz, 1H), 2.99 (dtd, J=12.5, 8.2, 4.2 Hz, 1H), 2.92-2.71 (m, 2H), 2.45 (s, 4H).

### Example 44: Synthesis of 5-(2-aminopyrimidin-5-yl)ethynyl)-1-methyl-3-(6-phenyl-5,6-dihydrocyclopenta[c]pyrazol-2 (4H)-yl)pyridine-2 (1H)-one

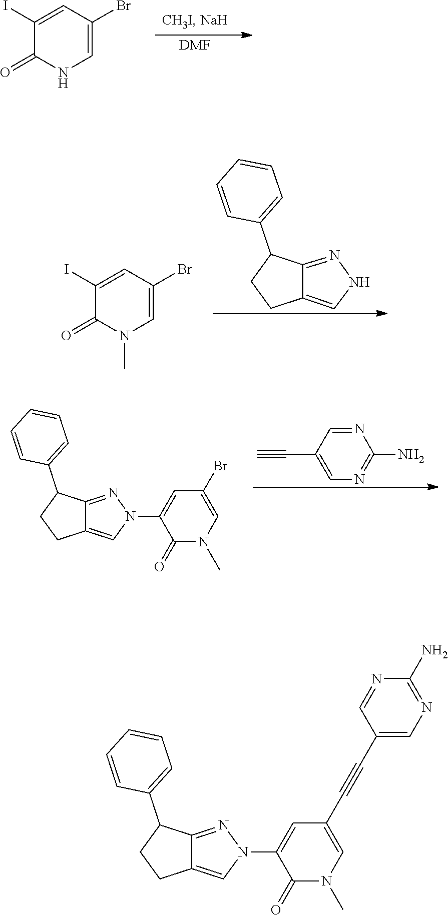

### Step 1: Synthesis of 5-bromo-3-iodo-1-methylpyridin-2 (1H)-one

5-bromo-3-iodopyridine-2 (1H)-one (3.0 g, 10 mmol) was dissolved in N,N-dimethylformamide (30 ml). To the resulting solution was added NaH (600 mg, 15 mmol) at 0° C. The mixture was reacted under nitrogen protection at 0° C. for 1 hour. iodomethane (2.8 g, 20 mmol) was slowly added dropwise to the reaction mixture. The resulting reaction mixture was stirred at room temperature for 1 hour. The reaction mixture was poured into ice-water (50 mL) to separate out a precipate, and filtered to produce 5-bromo-3-iodo-1-methylpyridin-2 (1H)-one (1.8 g) as a yellow solid in a yield of 57.3%. LC-MS (m/z): 314.3[M+H]⁺.

### Step 2 and Step 3: Synthesis of 5-bromo-1-methyl-3-(6-phenyl-5,6-dihydrocyclopenta[c]pyrazol-2 (4H)-yl)pyridine-2 (1H)-one and 5-(2-aminopyrimidin-5-yl)ethynyl)-1-methyl-3-(6-phenyl-5,6-dihydrocyclopenta[c]pyrazol-2 (4H)-yl)pyridine-2 (1H)-one

Example 44 was synthesized with reference to the synthesis of Example 1 to produce an off-white solid (36 mg) in a yield of 33%. LC-MS (m/z): 409.3[M+H]⁺. ¹H NMR (400 MHz, cdcl3) δ 7.60 (d, J=1.4 Hz, 2H), 7.44 (t, J=1.8 Hz, 1H), 7.34 (t, J=1.8 Hz, 1H), 7.15 (d, J=1.4 Hz, 1H), 6.54-6.46 (m, 4H), 6.40 (dt, J=5.0, 3.6 Hz, 1H), 4.41 (s, 2H), 3.53 (ddd, J=8.3, 6.8, 1.4 Hz, 1H), 3.29 (d, J=1.3 Hz, 3H), 2.15 (ddtd, J=12.4, 8.3, 4.1, 1.3 Hz, 1H), 2.08-1.88 (m, 2H), 1.62 (dddd, J=12.6, 8.1, 6.4, 1.4 Hz, 1H).

### Example 45: Synthesis of 5-(5-(6-(3-fluorophenyl)-5,6-dihydrocyclopenta[c]pyrazol-2 (4H)-yl)pyridin-3-yl)ethynyl)pyrimidin-2-amine

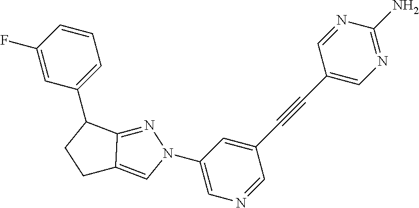 [SMILES: Nc1ncc(C#Cc2cncc(-n3cc4c(n3)C(c3cccc(F)c3)CC4)c2)cn1]

Example 45 was synthesized with reference to the synthesis of Example 1 to produce a white solid (45 mg) in a yield of 9.8%, MS(ESI): m/z 397.1 (M+H)+. ¹H NMR (400 MHz, CDCl₃) δ 8.90 (brs, 2H), 8.49 (s, 2H), 8.16 (s, 1H), 7.67 (s, 1H), 7.34-7.26 (m, 1H), 7.11 (d, J=7.6 Hz, 1H), 7.02 (d, J=10.4 Hz, 1H), 6.93 (t, J=8.4 Hz, 1H), 5.30 (s, 2H), 4.37 (t, J=7.6 Hz, 1H), 3.05-2.94 (m, 1H), 2.91-2.72 (m, 2H), 2.47-2.34 (m, 1H).

### Example 46: Synthesis of (S)-5-(5-(6-(3-fluorophenyl)-5,6-dihydrocyclopenta[c]pyrazol-2 (4H)-yl)pyridin-3-yl)ethynyl)pyrimidin-2-amine

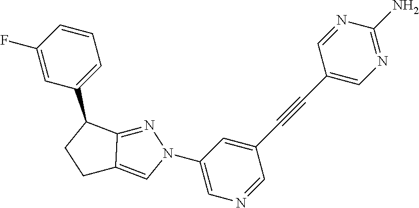 [SMILES: Nc1ncc(C#Cc2cncc(-n3cc4c(n3)[C@H](c3cccc(F)c3)CC4)c2)cn1]

Synthesis of Example 46: Preparative chiral resolution of Example 45 with SFC Thar prep 80 to produce a white solid (5 mg) in a yield of 7%. ¹H NMR (400 MHz, CDCl₃) δ 8.86 (d, J=2.4 Hz, 1H), 8.57 (d, J=1.6 Hz, 1H), 8.48 (s, 2H), 8.17 (dd, J=2.4, 1.6 Hz, 1H), 7.67 (s, 1H), 7.32-7.27 (m, 1H), 7.11 (d, J=7.6 Hz, 1H), 7.05-6.98 (m, 1H), 6.96-6.89 (m, 1H), 5.63 (s, 2H), 4.37 (t, J=7.6 Hz, 1H), 3.04-2.94 (m, 1H), 2.91-2.75 (m, 2H), 2.49-2.36 (m, 1H). LC-MS (m/z): 397.3[M+H]⁺.

### Example 47: Synthesis of (R)-5-((5-(6-(3-fluorophenyl)-5,6-dihydrocyclopenta[c]pyrazol-2 (4H)-yl)pyridine-3-yl)ethynyl)pyrimidin-2-amine

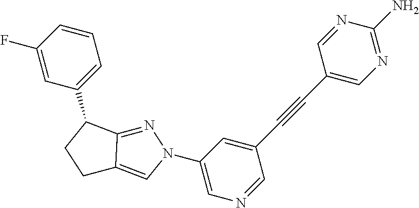 [SMILES: Nc1ncc(C#Cc2cncc(-n3cc4c(n3)[C@@H](c3cccc(F)c3)CC4)c2)cn1]

Synthesis of Example 47: Preparative chiral resolution of Example 45 with SFC Thar prep 80 to produce a white solid (34 mg) in a yield of 45%. ¹H NMR (400 MHz, DMSO-d₆) δ 8.98 (d, J=2.4 Hz, 1H), 8.51 (d, J=1.6 Hz, 1H), 8.45 (s, 2H), 8.34 (s, 1H), 8.23 (t, J=2.0 Hz, 1H), 7.35-7.27 (m, 1H), 7.23 (s, 2H), 7.18-7.01 (m, 3H), 4.32 (t, J=7.6 Hz, 1H), 2.75-2.61 (m, 3H), 2.31-2.24 (m, 1H). LC-MS (m/z): 397.3[M+H]⁺.

### Example 48: Synthesis of 5-(5-(6-(3,5-difluorophenyl)-5,6-dihydrocyclopenta[c]pyrazol-2 (4H)-yl)pyridin-3-yl)ethynyl)pyrimidin-2-amine

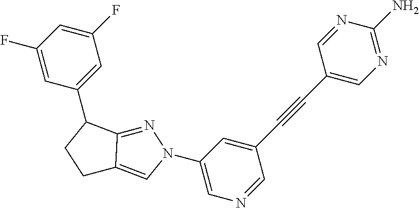 [SMILES: Nc1ncc(C#Cc2cncc(-n3cc4c(n3)C(c3cc(F)cc(F)c3)CC4)c2)cn1]

Example 48 was synthesized with reference to the synthesis of Example 1 to produce a white solid (15 mg) in a yield of 6%. LCMS (m/z): 415.1 (M+H)⁺. ¹H NMR (400 MHz, CDCl₃) δ 8.58 (d, J=2.4 Hz, 1H), 8.48 (s, 1H), 8.44 (s, 2H), 7.89 (t, J=2.0 Hz, 1H), 7.57 (s, 1H), 6.70-6.54 (m, 3H), 5.33 (s, 2H), 4.51 (dd, J=8.4, 3.6 Hz, 1H), 3.27-3.15 (m, 1H), 2.90-2.77 (m, 1H), 2.77-2.67 (m, 1H), 2.54-2.43 (m, 1H).

### Example 49: Synthesis of (R)-5-((5-(6-(3,5-difluorophenyl)-5,6-dihydrocyclopenta[c]pyrazol-2 (4H)-yl)pyridin-3-yl)ethynyl)pyrimidin-2-amine

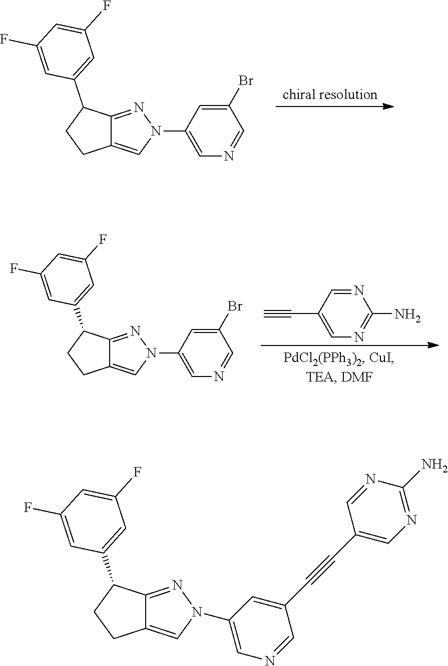

### Step 1: Synthesis of (R)-2-(5-bromopyridin-3-yl)-6-(3,5-difluorophenyl)-2,4,5,6-tetrahydrocyclopenta[c]pyrazole

2-(5-bromopyridin-3-yl)-6-(3,5-difluorophenyl)-2,4,5,6-tetrahydrocyclopenta[c]pyrazole was chirally resolved to produce (R)-2-(5-bromopyridin-3-yl)-6-(3,5-difluorophenyl)-2,4,5,6-tetrahydrocyclopenta[c]pyrazole (20 mg) as a white solid, LCMS (m/z): 377.2 (M+H)⁺.

### Step 2: Synthesis of (R)-5-((5-(6-(3,5-difluorophenyl)-5,6-dihydrocyclopenta[c]pyrazol-2 (4H)-yl)pyridin-3-yl)ethynyl)pyrimidin-2-amine

(R)-2-(5-bromopyridin-3-yl)-6-(3,5-difluorophenyl)-2,4,5,6-tetrahydrocyclopenta[c]pyrazole (20 mg, 0.053 mmol), 5-ethynylpyrimidin-2-amine (8 mg, 0.067 mmol), Pd(PPh₃)₂Cl₂ (10 mg, 0.01 mmol) and cuprous iodide (3 mg, 0.011 mmol) were added to N,N-dimethylformamide (1 mL) and triethylamine (1 mL). The atmosphere was vacuumed and replaced with nitrogen gas three times. The mixture was warmed up to 100° C., stirred for 16 hours, and cooled to room temperature. The solution was rotary evaporated to dryness. The residue was dissolved in dichloromethane (5 mL), and filtered. The filtrate was loaded to silica gel column and purified with silica gel column chromatography (dichloromethane:methanol=20:1) to produce a white solid compound (R)-5-((5-(6-(3,5-difluorophenyl)-5,6-dihydrocyclopenta[c]pyrazol-2 (4H)-yl)pyridin-3-yl)ethynyl)pyrimidin-2-amine (8 mg) in a yield of 37%, LCMS (m/z): 415.1 (M+H)⁺. ¹H NMR (400 MHz, CDCl₃) δ 8.85 (s, 2H), 8.48 (s, 2H), 8.15 (s, 1H), 7.68 (s, 1H), 6.89-6.81 (m, 2H), 6.73-6.62 (m, 1H), 5.41 (s, 2H), 4.35 (t, J=7.6 Hz, 1H), 3.05-2.92 (m, 1H), 2.90-2.73 (m, 2H), 2.45-2.33 (m, 1H). LC-MS (m/z): 415.4 [M+H]⁺.

### Example 50: Synthesis of (S)-5-((5-(6-(3,5-difluorophenyl)-5,6-dihydrocyclopenta[c]pyrazol-2 (4H)-yl)pyridin-3-yl)ethynyl)pyrimidin-2-amine

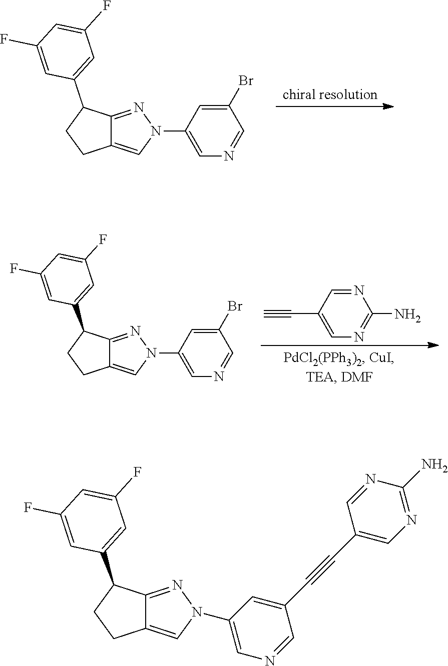

### Step 1: Synthesis of (S)-2-(5-bromopyridin-3-yl)-6-(3,5-difluorophenyl)-2,4,5,6-tetrahydrocyclopenta[c]pyrazole

2-(5-bromopyridin-3-yl)-6-(3,5-difluorophenyl)-2,4,5,6-tetrahydrocyclopenta[c]pyrazole was chirally resolved to produce (S)-2-(5-bromopyridin-3-yl)-6-(3,5-difluorophenyl)-2,4,5,6-tetrahydrocyclopenta[c]pyrazole (40 mg) as a white solid, LCMS (m/z): 377.3 (M+H)⁺.

### Step 2: Synthesis of (S)-5-((5-(6-(3,5-difluorophenyl)-5,6-dihydrocyclopenta[c]pyrazol-2 (4H)-yl)pyridin-3-yl)ethynyl)pyrimidin-2-amine

Example 50 was synthesized with reference to the synthesis of Example 1 to produce a white solid (15 mg) in a yield of 35%. LCMS (m/z): 415.1 (M+H)⁺. ¹H NMR (400 MHz, CDCl₃) δ 8.87 (s, 1H), 8.60 (s, 1H), 8.48 (s, 2H), 8.14 (s, 1H), 7.68 (s, 1H), 6.92-6.80 (m, 2H), 6.68 (tt, J=9.2, 2.4 Hz, 1H), 5.30 (s, 2H), 4.35 (t, J=7.6 Hz, 1H), 3.04-2.94 (m, 1H), 2.91-2.73 (m, 2H), 2.45-2.35 (m, 1H).

### Example 51: Synthesis of 5-(5-(6-phenyl-4H-furo[3,4-c]pyrazol-2 (6H)-yl)pyridin-3-ethynyl)pyrimidin-2-amine

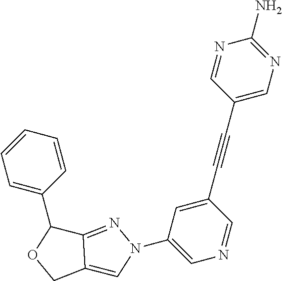 [SMILES: Nc1ncc(C#Cc2cncc(-n3cc4c(n3)C(c3ccccc3)OC4)c2)cn1]

Example 51 was synthesized with reference to the synthesis of Example 1 to produce a white solid (15.0 mg) in a yield of 20.0%. LC-MS (m/z): 381.3[M+H]⁺. ¹H NMR (400 MHz, chloroform-d) δ 9.03 (d, J=2.4 Hz, 1H), 8.59 (d, J=1.6 Hz, 1H), 8.49 (d, J=5.6 Hz, 3H), 8.32-8.28 (m, 1H), 7.45-7.30 (m, 5H), 7.28 (s, 2H), 6.08 (s, 1H), 5.11 (d, J=11.6 Hz, 1H), 5.00 (d, J=11.6 Hz, 11H).

### Example 52: Synthesis of 2-(5-((2-aminopyrimidin-5-yl)ethynyl)pyridin-3-yl)-7-phenyl-2,5,6,7-tetrahydro-3H-pyrrolo[2,1-c][1,2,4]triazole-3-one

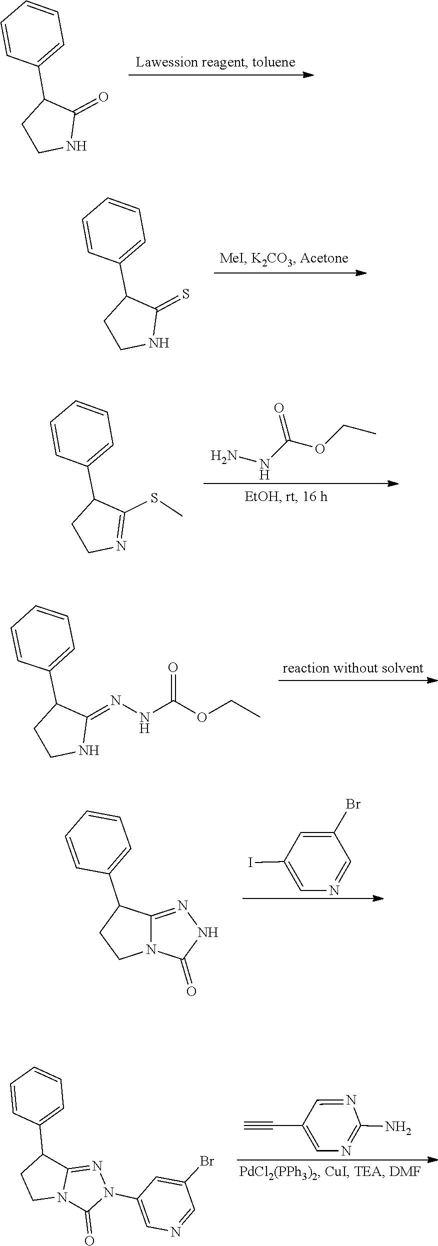

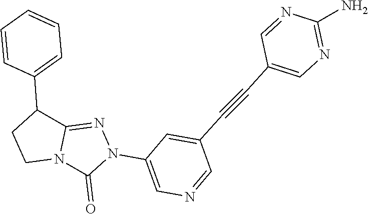 [SMILES: Nc1ncc(C#Cc2cncc(-n3nc4n(c3=O)CCC4c3ccccc3)c2)cn1]

### Step 1: Synthesis of 3-phenylpyrrolidine-2-thione

3-phenylpyrrolidin-2-one (1.1 g, 6.8 mmol), and Lawession reagent (5.4 g, 36.5 mmol) were added to toluene (20 ml). The atmosphere was vacuumed and replaced with nitrogen gas three times. The reaction mixture was warmed up to 80° C., stirred for 2 hours, and cooled to room temperature. The solution was rotary evaporated to dryness, and a small amount of ethyl acetate was added. The mixture was stirred, and the resulting solid was filtered to produce a white solid compound 3-phenylpyrrolidine-2-thione (1.1 g) in a yield of 82%, LCMS (m/z): 178.1 (M+H)⁺.

### Step 2: Synthesis of 5-(methylthio)-4-phenyl-3,4-dihydro-2H-pyrrole

Compound 3-phenylpyrrolidin-2-thione (1.1 g, 6.2 mmol), iodomethane (1.3 g, 9.3 mmol) and potassium carbonate (2.5 g, 18.6 mmol) were added to acetone (20 mL). The mixture was stirred at room temperature overnight, and water (100 mL) was added. The resulting mixture was extracted with ethyl acetate (20 mL) three times. The organic phases were combined, washed with water (20 mL) and saturated brine (20 mL) in turn, dried over anhydrous Na₂SO₄, filtered, and rotary evaporated to dryness. The residue was loaded to silica gel column and purified with silica gel column chromatography (petroleum ether:ethyl acetate=20:1) to produce a white solid compound 5-(methylthio)-4-phenyl-3,4-dihydro-2H-pyrrole (800 mg) in a yield of 68%, LCMS (m/z): 192.0 (M+H)⁺.

### Step 3: Synthesis of ethyl (Z)-2-(3-phenylpyrrolidin-2-ylidene)hydrazine-1-carboxylate

Compound 5-(methylthio)-4-phenyl-3,4-dihydro-2H-pyrrole (800 mg, 4.18 mmol) and ethoxycarbohydrazide (870.7 mg, 8.3 mmol) were added to ethanol (20 ml). The mixture was warmed to 80° C., stirred for 48 hours, and cooled to room temperature. The solution was rotary evaporated to dryness. The residue was loaded to silica gel column and purified with silica gel column chromatography (petroleum ether:ethyl acetate=1:1) to produce a white solid compound ethyl (Z)-2-(3-phenylpyrrolidin-2-ylidene)hydrazine-1-carboxylate (500 mg) in a yield of 44%, LCMS (m/z): 248.0 (M+H)⁺.

### Step 4: Synthesis of 7-phenyl-2,5,6,7-tetrahydro-3H-pyrrolo[2,1-c][1,2,4]triazole-3-one

Compound Ethyl (Z)-2-(3-phenylpyrrolidin-2-ylidene)hydrazine-1-carboxylate (500 mg, 2.5 mmol) was added to a single necked bottle, and warmed up to 150° C., raw materials appeared to melt. At this temperature, the mixture was stirred at this temperature for 2 hours, and cooled to room temperature. The residue was loaded to silica gel column and purified with silica gel column chromatography (petroleum ether:ethyl acetate=8:1) to produce a white solid compound 7-phenyl-2,5,6,7-tetrahydro-3H-pyrrolo[2,1-c][1,2,4]triazole-3-one (350 mg) in a yield of 77%, LCMS (m/z): 202.0 (M+H)⁺.

### Step 5: Synthesis of 2-(5-bromopyridin-3-yl)-7-phenyl-2,5,6,7-tetrahydro-3H-pyrrolo[2,1-c][1,2,4]triazole-3-one

Compound 7-phenyl-2,5,6,7-tetrahydro-3H-pyrrolo[2,1-c][1,2,4]triazole-3-one (240 mg, 1.19 mmol), 3-bromo-5-iodopyridine (507.9 mg, 1.78 mmol), (1R,2R)—N¹,N²-dimethylcyclohexane-1,2-diamine (33.9 mg, 0.23 mmol), cuprous iodide (45.4 mg, 0.23 mmol) and potassium phosphate (757.5 mg, 3.57 mmol) were added to 1,4-dioxane (10 mL). The mixture was blowed with nitrogen, heated under microwave to 120° C., stirred for 3 hours, filtered, and rotary evaporated to dryness. The residue was loaded to silica gel column and purified with silica gel column chromatography (petroleum ether:ethyl acetate=1:1) to produce a white solid compound 2-(5-bromopyridin-3-yl)-7-phenyl-2,5,6,7-tetrahydro-3H-pyrrolo[2,1-c][1,2,4]triazole-3-one (150 mg) in a yield of 32%, LCMS (m/z): 357.0 (M+H)⁺.

### Step 6: Synthesis of 2-(5-((2-aminopyrimidin-5-yl)ethynyl)pyridin-3-yl)-7-phenyl-2,5,6,7-tetrahydro-3H-pyrrolo[2,1-c][1,2,4]triazole-3-one

2-(5-bromopyridin-3-yl)-7-phenyl-2,5,6,7-tetrahydro-3H-pyrrolo[2,1-c][1,2,4]triazole-3-one (100 mg, 0.28 mmol), 5-ethynylpyrimidin-2-amine (40 mg, 0.34 mmol), PdCl₂(PPh₃)₂ (40 mg, 0.06 mmol) and cuprous iodide (10 mg, 0.04 mmol) were added to N,N-dimethylformamide (1.5 mL) and triethylamine (1.5 mL). The atmosphere was vacuumed and replaced with nitrogen gas three times. The mixture was warmed up to 120° C., stirred for 2 hours, and cooled to room temperature. The solution was rotary evaporated to dryness. The residue was dissolved in dichloromethane (5 mL), and the resulting mixture was filtered. The filtrate was loaded to silica gel column and purified with silica gel column chromatography (dichloromethane:methanol=20:1) to produce a white solid compound 2-(5-((2-aminopyrimidin-5-yl)ethynyl)pyridin-3-yl)-7-phenyl-2,5,6,7-tetrahydro-3H-pyrrolo[2,1-c][1,2,4]triazole-3-one (50 mg) in a yield of 45%, 5 LCMS (m/z): 396.1 (M+H)⁺. ¹H NMR (400 MHz, DMSO) δ 9.05 (s, 1H), 8.51 (s, 1H), 8.49 (s, 2H), 8.27 (t, J=2.0 Hz, 1H), 7.48-7.43 (m, 2H), 7.42-7.36 (m, 2H), 7.35-7.29 (m, 1H), 7.27 (brs, 2H), 4.56 (t, J=8.4 Hz, 1H), 3.98-3.91 (m, 1H), 3.86-3.77 (m, 1H), 3.04-2.94 (m, 1H), 2.45-2.35 (m, 1H).

### Example 53: Synthesis of 3-((3-(4-phenyl-5,6-dihydrocyclopenta[d][1,2,3]triazol-2 (4H)-yl)phenyl)ethynyl)imidazo[1,2-b]pyridazine

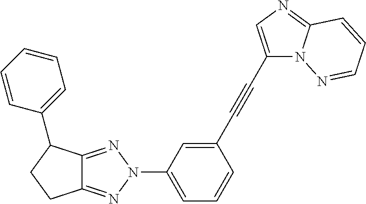 [SMILES: C(#Cc1cnc2cccnn12)c1cccc(-n2nc3c(n2)C(c2ccccc2)CC3)c1]

Example 53 was synthesized with reference to the synthesis of Example 2 to produce a light-yellow solid (21 mg) in a yield of 51.2%. LC-MS (m/z): 403.4 [M+H]⁺. ¹H NMR (400 MHz, chloroform-d) δ 8.63 (d, J=4.4 Hz, 1H), 8.36-8.27 (m, 2H), 8.07 (ddd, J=8.0, 2.4, 1.2 Hz, 11H), 7.57-7.51 (m, 11H), 7.47 (ddd, J=8.2, 7.6, 0.4 Hz, 1H), 7.37-7.28 (m, 4H), 7.27-7.23 (m, 3H), 4.49-4.42 (m, 1H), 3.17-2.89 (m, 3H), 2.56 (ddt, J=12.8, 8.6, 6.8 Hz, 1H).

### Example 54: Synthesis of 5-((5-(4-phenyl-5,6-dihydrocyclopenta[d][1,2,3]triazol-2 (4H)-yl)pyridine-3-yl)ethynyl)pyrimidin-2-amine

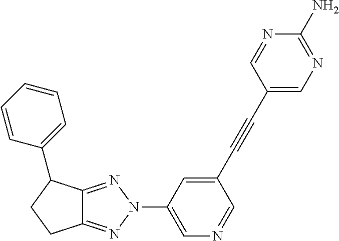 [SMILES: Nc1ncc(C#Cc2cncc(-n3nc4c(n3)C(c3ccccc3)CC4)c2)cn1]

Example 54 was synthesized with reference to the synthesis of Example 2 to produce a light-yellow solid (4.6 mg) in a yield of 15.6%. LC-MS (m/z): 380.4 [M+H]⁺. ¹H NMR (400 MHz, chloroform-d) δ9.24 (s, 1H), 8.62 (s, 1H), 8.47 (s, 2H), 8.40 (s, 1H), 7.37-7.31 (m, 2H), 7.30-7.25 (m, 3H), 5.33 (s, 2H), 4.45 (dd, J=8.3, 7.1 Hz, 1H), 3.18-2.87 (m, 3H), 2.62-2.48 (m, 1H).

### Example 55: (S)-5-((5-(4-phenyl-5,6-dihydrocyclopenta[d][1,2,3]triazol-2 (4H)-yl)pyridin-3-yl)ethynyl)pyrimidin-2-amine

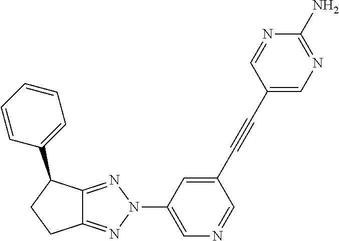 [SMILES: Nc1ncc(C#Cc2cncc(-n3nc4c(n3)[C@H](c3ccccc3)CC4)c2)cn1]

Synthesis of Example 55: Chiral resolution of Example 54 to produce a 5 white solid (40 mg), ee value: 99.86%. LC-MS (m/z): 380.4 [M+H]⁺. ¹H NMR (400 MHz, chloroform-d) δ 9.35 (s, 1H), 8.80 (s, 1H), 8.49 (s, 2H), 8.41 (s, 1H), 7.40-7.32 (m, 2H), 7.31-7.27 (m, 3H), 5.32 (s, 2H), 4.46 (t, J=7.6 Hz, 1H), 3.16-3.11 (m 1H), 3.10-2.90 (m, 2H), 2.62-2.53 (m, 1H).

### Example 56: Synthesis of (R)-5-((5-(4-phenyl-5,6-dihydrocyclopenta[d][1,2,3]triazol-2 (4H)-yl)pyridin-3-yl)ethynyl)pyrimidin-2-amine

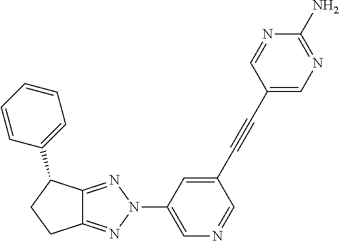 [SMILES: Nc1ncc(C#Cc2cncc(-n3nc4c(n3)[C@@H](c3ccccc3)CC4)c2)cn1]

Synthesis of Example 56: Chiral resolution of Example 54 to produce a white solid (35 mg), ee value: 99.96%. LC-MS (m/z): 380.3 [M+H]⁺. ¹H NMR (400 MHz, chloroform-d) δ 9.25 (s, 1H), 8.63 (s, 1H), 8.48 (s, 2H), 8.41 (dd, J=2.4, 1.8 Hz, 1H), 7.38-7.32 (m, 2H), 7.32-7.26 (m, 3H), 5.33 (s, 2H), 4.46 (t, J=7.6 Hz, 1H), 3.17-3.07 (m, 1H), 3.07-2.90 (m, 2H), 2.62-2.52 (m, 1H).

### Example 57: Synthesis of 4-((2-aminopyrimidin-5-yl)ethynyl)-1-methyl-6-(4-phenyl-5,6-dihydrocyclopenta[d][1,2,3]triazol-2 (4H)-yl)pyridine-2 (1H)-one

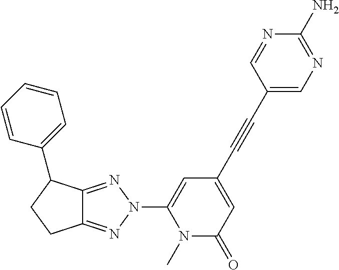 [SMILES: Cn1c(-n2nc3c(n2)C(c2ccccc2)CC3)cc(C#Cc2cnc(N)nc2)cc1=O]

Example 57 was synthesized with reference to the synthesis of Example 2 to produce an off-white solid (41 mg) in a yield of 41.4%. LC-MS (m/z): 410.3 [M+H]⁺. ¹H NMR (400 MHz, chloroform-d) δ 8.43 (s, 2H), 7.39-7.32 (m, 2H), 7.28 (d, J=7.5 Hz, 3H), 6.77 (d, J=1.8 Hz, 1H), 6.45 (d, J=1.8 Hz, 1H), 5.36 (s, 2H), 4.47 (t, J=7.8 Hz, 1H), 3.47 (s, 3H), 3.15 (dp, J=13.1, 4.3 Hz, 1H), 3.07-2.87 (m, 2H), 2.60 (ddt, J=12.9, 8.7, 7.3 Hz, 1H).

### Example 58: Synthesis of 5-((6-methyl-5-(4-phenyl-5,6-dihydrocyclopenta[d][1,2,3]triazol-2 (4H)-yl)pyridin-3-yl)ethynyl)pyrimidin-2-amine

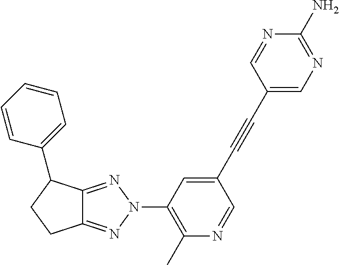 [SMILES: Cc1ncc(C#Cc2cnc(N)nc2)cc1-n1nc2c(n1)C(c1ccccc1)CC2]

Example 58 was synthesized with reference to the synthesis of Example 2 to produce a light-yellow solid (12.7 mg) in a yield of 30.4%. LC-MS (m/z): 394.3[M+H]⁺. ¹H NMR (400 MHz, chloroform-d) δ 8.63 (d, J=1.9 Hz, 1H), 8.46 (s, 2H), 8.10 (d, J=2.0 Hz, 1H), 7.40-7.27 (m, 5H), 5.32 (s, 2H), 4.48 (t, J=7.7 Hz, 1H), 3.23-2.89 (m, 4H), 2.79 (s, 3H), 2.68-2.41 (m, 2H).

### Example 59: Synthesis of 4-((2-aminopyrimidin-5-yl)ethynyl)-6-(4-(5-fluoropyridin-3-yl)-5,6-dihydrocyclopenta[d][1,2,3]triazol-2 (4H)-yl)-1-methylpyridin-2 (1H)-one

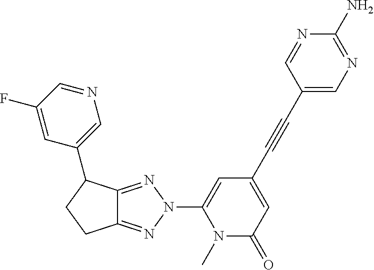 [SMILES: Cn1c(-n2nc3c(n2)C(c2cncc(F)c2)CC3)cc(C#Cc2cnc(N)nc2)cc1=O]

Example 59 was synthesized with reference to the synthesis of Example 2 to produce a white solid (92 mg) in a yield of 36.4%.

LC-MS (m/z): 429.3[M+H]⁺. ¹H NMR (400 MHz, DMSO) δ 8.56-8.40 (m, 4H), 7.72 (dt, J=10.0, 2.5 Hz, 1H), 7.36 (s, 2H), 6.68 (d, J=1.8 Hz, 1H), 6.57 (d, J=1.8 Hz, 1H), 4.68 (t, J=8.0 Hz, 1H), 3.30 (s, 3H), 3.14 (dtt, J=14.7, 9.8, 5.0 Hz, 1H), 3.07-2.88 (m, 2H), 2.55 (d, J=7.7 Hz, 1H).

### Example 60: Synthesis of 5-((2-aminopyrimidin-5-yl)ethynyl)-3-(4-phenyl-5,6-dihydrocyclopenta[d][1,2,3]triazol-2 (4H)-yl)pyridine-2 (1H)-one

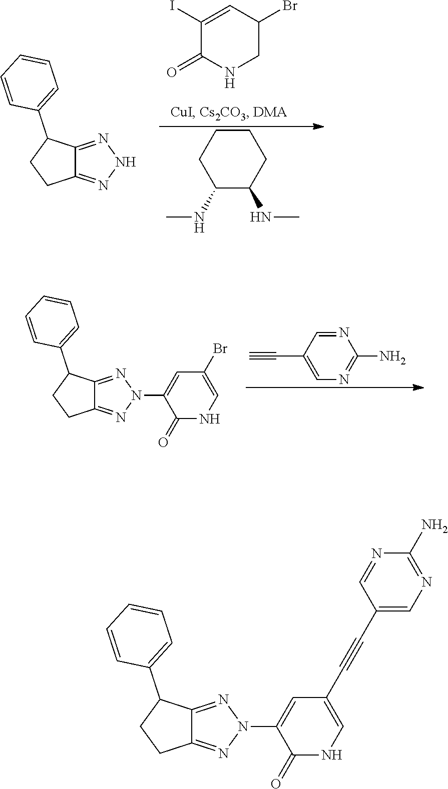

### Step 1: Synthesis of 5-bromo-3-(4-phenyl-5,6-dihydrocyclopenta[d][1,2,3]triazol-2 (4H)-yl)pyridine-2 (1H)-one

Cuprous iodide (50 mg, 0.263 mmol), (1R,2R)—N¹,N²-dimethylcyclohexane-1,2-diamine (78 mg, 0.548 mmol) and cesium carbonate (2.6 g, 8 mmol) were added to a solution of 4-phenyl-2,4,5,6-tetrahydrocyclopenta[d][1,2,3]triazole (500 mg, 2.688 mmol) and 5-bromo-3-iodopyridine-2 (1H)-one (1.2 g, 4 mmol) in N,N-dimethylacetamide (5 mL). The mixture was reacted under nitrogen protection at 110° C. overnight. Water (10 mL) was added to the reaction mixture. The resulting reaction mixture was extracted with ethyl acetate (3×30 mL). The organic phases were combined, washed with brine, dried over anhydrous Na₂SO₄, filtered, and rotary evaporated to dryness. The residue was purified with silica gel column (petroleum ether/ethyl acetate=1/1) to produce 5-bromo-3-(4-phenyl-5,6-dihydrocyclopenta[d][1,2,3]triazole-2 (4H)-yl)pyridine-2 (1H)-one (300 mg) as a white solid in a yield of 31.2%. LC-MS (m/z): 358.3[M+H]⁺.

### Step 2: Synthesis of 5-((2-aminopyrimidin-5-yl)ethynyl)-3-(4-phenyl-5,6-dihydrocyclopenta[d][1,2,3]triazol-2 (4H)-yl)pyridine-2 (1H)-one

Example 60 was synthesized with reference to the synthesis of Example 2 to produce an off-white solid (12 mg) in a yield of 7.4%. LC-MS (m/z): 396.3[M+H]⁺. ¹H NMR (400 MHz, CDCl₃) δ 8.83 (d, J=15.3 Hz, 1H), 8.48 (d, J=42.7 Hz, 1H), 8.05 (d, J=46.0 Hz, 1H), 7.68-7.48 (m, 1H), 7.27 (d, J=32.6 Hz, 5H), 4.42-4.24 (m, 1H), 3.06-2.62 (m, 3H), 2.44 (d, J=12.9 Hz, 1H).

### Example 61: Synthesis of (S)-5-(2-(5-(6-phenyl-5,6-dihydrocyclopenta[c]pyrazol-2 (4H)-yl)pyridin-3-yl)ethyl)pyrimidin-2-amine

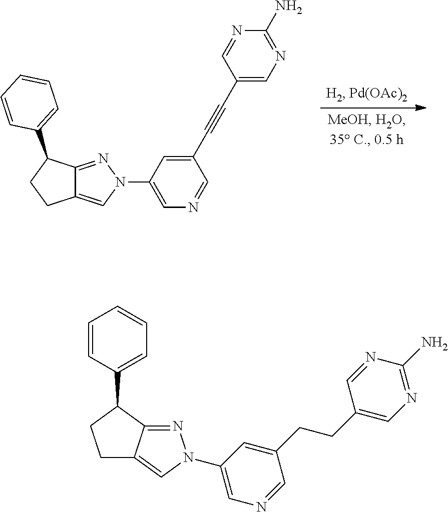 [SMILES: Nc1ncc(C#Cc2cncc(-n3cc4c(n3)[C@H](c3ccccc3)CC4)c2)cn1 ; Nc1ncc(CCc2cncc(-n3cc4c(n3)[C@H](c3ccccc3)CC4)c2)cn1]

(S)-5-(5-(6-phenyl-5,6-dihydrocyclopenta[c]pyrazol-2 (4H)-yl)pyridin-3-yl)ethynyl)pyrimidin-2-amine (100 mg, 264.24 μmol) was dissolved in methanol (10 mL). Water (2 mL) and palladium acetate (6 mg, 26.42 μmol) were added. The atmosphere was replaced with hydrogen gas three times. The resulting mixture was reacted under a condition of 18 psi hydrogen gas at 35° C. for 0.5 hours. LCMS detection indicated the reaction product formation. Ethyl acetate (50 mL) was added to dilute the reaction system. The resulting mixture was extracted with water (25 mL×3) and saturated brine (25 mL) in turn, and dried over anhydrous Na₂SO₄. The solvent was removed by rotary evaporation to dryness with a water pump. The resulting crude product was purified with preparative HPLC to produce as a white solid (S)-5-(2-(5-(6-phenyl-5,6-dihydrocyclopenta[c]pyrazol-2 (4H)-yl)pyridin-3-yl)ethyl)pyrimidin-2-amine (18 mg) in a yield of 18%. ¹H NMR (400 MHz, DMSO) δ(ppm): 8.84 (d, J=2.5 Hz, 1H), 8.30-8.24 (m, 2H), 8.08 (s, 2H), 8.03 (t, J=2.2 Hz, 1H), 7.37-7.17 (m, 5H), 6.38 (s, 2H), 4.33 (dd, J=8.2, 7.0 Hz, 1H), 2.99-2.65 (m, 6H), 2.36-2.25 (m, 1H), 2.00 (q, J=7.0, 6.6 Hz, 1H). LCMS (m/z): 383.1[M+H]⁺.

### Example 62: Synthesis of 4-(2-aminopyrimidin-5-yl)ethynyl)-1-methyl-6-(4-(pyridin-3-yl)-5,6-dihydrocyclopenta[d][1,2,3]triazol-2 (4H)-yl)pyridine-2 (1H)-one

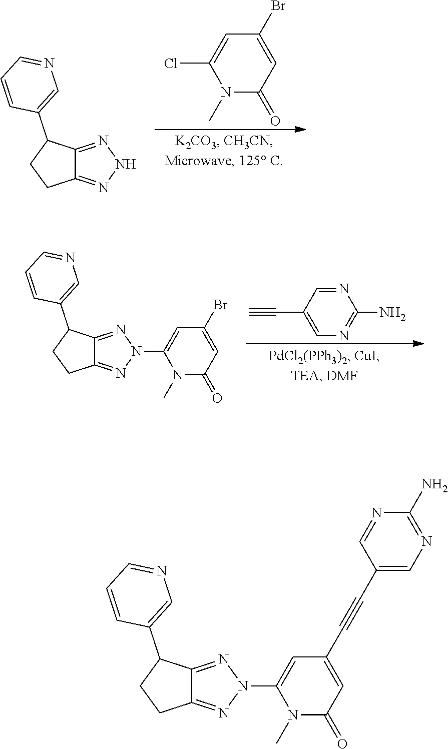

### Step 1: Synthesis of 4-bromo-1-methyl-6-(4-(pyridin-3-yl)-5,6-dihydrocyclopenta[d][1,2,3]triazol-2 (4H)-yl)pyridine-2 (1H)-one

Compound 4-(pyridin-3-yl)-2,4,5,6-tetrahydrocyclopenta[d][1,2,3]triazole (200 mg, 1.1 mmol), 4-bromo-6-chloro-1-methylpyridin-2 (1H)-one (245 mg, 1.1 mmol) and potassium carbonate (304 mg, 2.2 mmol) were added to acetonitrile. The mixture was reacted under microwave at 125° C. for 0.5 hours, and filtered. The filtrate was loaded to silica gel column and purified with silica gel column chromatography (dichloromethane:methanol=100:1) to produce a white solid compound 4-bromo-1-methyl-6-(4-(pyridin-3-yl)-5,6-dihydrocyclopenta[d][1,2,3]triazol-2 (4H)-yl)pyridine-2 (1H)-one (150 mg) in a yield of 37%, LCMS (m/z): 373.0 (M+H)⁺.

### Step 2: Synthesis of 4-(2-aminopyrimidin-5-yl)ethynyl)-1-methyl-6-(4-(pyridin-3-yl)-5,6-dihydrocyclopenta[d][1,2,3]triazol-2 (4H)-yl)pyridine-2 (1H)-one

4-bromo-1-methyl-6-(4-(pyridin-3-yl)-5,6-dihydrocyclopenta[d][1,2,3]triazol-2 (4H)-yl)pyridine-2 (1H)-one (150 mg, 0.40 mmol), 5-ethynylpyrimidin-2-amine (48 mg, 0.40 mmol), PdCl₂(PPh₃)₂ (17 mg, 0.04 mmol) and cuprous iodide (10 mg, 0.04 mmol) were added to N,N-dimethylformamide (1.5 mL) and triethylamine (1.5 mL). The atmosphere was vacuumed and replaced with nitrogen gas three times. The mixture was warmed up to 100° C., stirred for 16 hours, and cooled to room temperature. The solution was rotary evaporated to dryness. The residue was dissolved in dichloromethane (5 mL), and filtered. The filtrate was loaded to silica gel column and purified with silica gel column chromatography (dichloromethane:methanol=20:1) to produce a white solid compound 4-(2-aminopyrimidin-5-yl)ethynyl)-1-methyl-6-(4-(pyridin-3-yl)-5,6-dihydrocyclopenta[d][1,2,3]triazol-2 (4H)-yl)pyridine-2 (1H)-one (20 mg) in a yield of 12%. LCMS (m/z): 410.3 (M+H)⁺. ¹H NMR (400 MHz, CDCl₃) δ 8.62 (s, 1H), 8.56 (s, 1H), 8.44 (s, 2H), 7.65 (d, J=7.6 Hz, 1H), 7.38-7.32 (m, 1H), 6.78 (d, J=2.0 Hz, 1H), 6.45 (d, J=2.0 Hz, 1H), 5.38 (s, 2H), 4.51 (t, J=7.6 Hz, 1H), 3.47 (s, 3H), 3.30-2.94 (m, 3H), 2.63-2.57 (m, 1H).

### Example 63: Synthesis of 5-((5-(4-(3,5-difluorophenyl)-5,6-dihydrocyclopenta[d][1,2,3]triazol-2 (4H)-yl)pyridin-3-yl)ethynyl)pyrimidin-2-amine

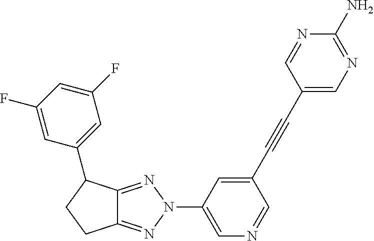 [SMILES: Nc1ncc(C#Cc2cncc(-n3nc4c(n3)C(c3cc(F)cc(F)c3)CC4)c2)cn1]

Example 63 was synthesized with reference to the synthesis of Example 2 to produce a white solid (15.0 mg) in a yield of 34.0%. LC-MS (m/z): 416.4 [M+H]⁺. ¹H NMR (400 MHz, DMSO-d₆) δ 9.14 (d, J=2.4 Hz, 1H), 8.67 (d, J=2.0 Hz, 1H), 8.50 (s, 1H), 8.39-8.33 (m, 1H), 7.26 (brs, 2H), 7.18-7.06 (m, 4H), 4.60 (t, J=7.6 Hz, 1H), 3.18-2.88 (m, 3H), 2.46-2.35 (m, 1H).

### Example 64: Synthesis of 5-((5-(4-cyclohexyl-5,6-dihydrocyclopenta[d][1,2,3]triazol-2 (4H)-yl)-6-methylpyridin-3-yl)ethynyl)pyrimidin-2-amine

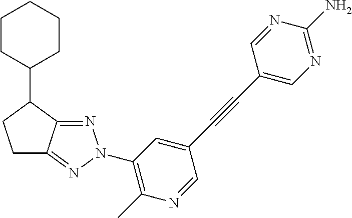 [SMILES: Cc1ncc(C#Cc2cnc(N)nc2)cc1-n1nc2c(n1)C(C1CCCCC1)CC2]

Example 64 was synthesized with reference to the synthesis of Example 2 to produce a white solid (80 mg) in a yield of 48.1%. LC-MS (m/z): 400.4 [M+H]⁺. ¹H NMR (400 MHz, chloroform-d) δ 8.61 (d, J=2.0 Hz, 1H), 8.47 (s, 2H), 8.06 (d, J=2.0 Hz, 1H), 5.27 (s, 2H), 3.04-2.94 (m, 1H), 2.91-2.79 (m, 2H), 2.77 (s, 3H), 2.74-2.59 (m, 1H), 2.37-2.26 (m, 1H), 2.11 (d, J=12.8 Hz, 1H), 1.83-1.67 (m, 5H), 1.58-1.45 (m, 1H), 1.33-1.12 (m, 3H), 1.11-0.98 (m, 1H).

### Example 65: 4-((2-aminopyrimidin-5-yl)ethynyl)-6-(4-cyclohexyl-5,6-dihydrocyclopenta[d][1,2,3]triazol-2 (4H)-yl)-1-methylpyridin-2 (1H)-one

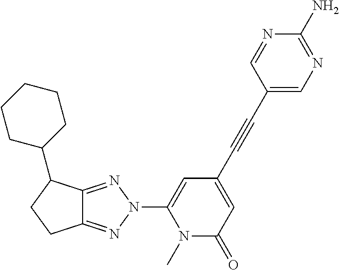 [SMILES: Cn1c(-n2nc3c(n2)C(C2CCCCC2)CC3)cc(C#Cc2cnc(N)nc2)cc1=O]

Example 65 was synthesized with reference to the synthesis of Example 2 to produce a white solid (100 mg) in a yield of 21.3%. LC-MS (m/z): 416.4 [M+H]⁺. ¹H NMR (400 MHz, chloroform-d) δ 8.44 (s, 2H), 6.76 (d, J=1.6 Hz, 1H), 6.43 (d, J=1.6 Hz, 1H), 5.38 (s, 2H), 3.45 (s, 3H), 3.04-2.94 (m, 1H), 2.92-2.77 (m, 2H), 2.76-2.64 (m, 1H), 2.40-2.27 (m, 1H), 2.11-2.03 (m, 1H), 1.90-1.83 (m, 1H), 1.81-1.74 (m, 3H), 1.72-1.65 (m, 1H), 1.57-1.44 (m, 1H), 1.32-1.11 (m, 3H), 1.10-0.97 (m, 1H).

### Example 66: Synthesis of 2-(5-((1H-pyrazole-4-yl)ethynyl)-2-methylpyridin-3-yl)-4-phenyl-2,4,5,6-tetrahydrocyclopenta[d][1,2,3]triazole

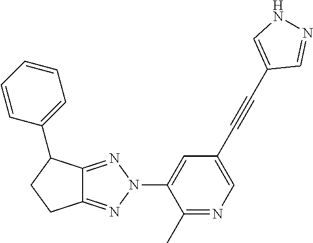 [SMILES: Cc1ncc(C#Cc2cn[nH]c2)cc1-n1nc2c(n1)C(c1ccccc1)CC2]

Example 66 was synthesized with reference to the synthesis of Example 2 to produce a light-yellow solid (60 mg) in a yield of 30.6%. LC-MS (m/z): 367.1 [M+H]⁺. ¹H NMR (400 MHz, DMSO-d₆) δ13.28 (s, 1H), 8.64 (s, 1H), 8.20 (s, 1H), 8.07 (s, 1H), 7.79 (s, 1H), 7.45-7.23 (m, 5H), 4.53 (t, J=7.6 Hz, 1H), 3.14-3.89 (m, 3H), 2.64 (s, 3H), 2.45-2.39 (m, 1H).

### Biological Assay

### Example 67: In Vitro Biological Activity Assay

In order to verify the inhibitory effect of the compounds of the present disclosure on programmed necrosis at the cellular level, a type of cells closely related to the RIP1 pathway, namely HT-29 human colon cancer cells, were selected. The used activation mode is a combination of tumor necrosis factor (TNFα), cysteine aspartate activator mimic (SmacM) and pan-caspase inhibitor Z_VAD FMK, and the cell viability was calculated by detecting chemiluminescence values, thus obtaining the biological activity of the compound inhibiting the programmed necrosis of cells.

### Assay Condition and Process:

### 1. Assay Materials

Cell line: HT-29 (ATCC® HTB-38™)

McCOY's 5A culture medium: Gibco, Cat No. 16600-082

Fetal bovine serum: Gibco, Cat No. 10099-141C

Trypsin: Gibco, Cat No. 25200-056

DMSO: Sigma, Cat No. 67-68-5, 1 L

96-well assay plate: Corning #3903

96-well compound dilution plate: Corning #3357

Inducer: TNFα: GenScript, Cat No. Z01001-50,

SmacM: Cat. No., HY-15989, MedChemExpress (MCE)

Z_VAD FMK: Target Mol, T6013

Cell Titer-Glo® Chemiluminescent Cell Viability Assay Kit: Promega, Cat No. G7573

Microplate reader (EnVision): PerkinElmer, 2105-0010

### 2. Cell Inoculation

2.1. The Growth Status of HT-29 Cells was Regularly Checked to Ensure their Normal Growth.
2.2. McCOY's 5A culture medium containing 10% FBS was preheated in a 37° C. water bath for 30 minutes. When the culture medium was ready for use, the cell passage and inoculation was performed in a Class II safety cabinet with aseptic technique.
2.3. In a T75 culture flask, when the growth density of cells reached the abundance of approximately 80%, the culture medium was aspirated and cells were washed twice with pre-warmed PBS.
2.4. 2-3 mL of trypsin (Gibco, catalog number 25200-056) was added and the T75 culture flask was transferred to a 37° C. incubator.
2.5. After 3-5 minutes, the side of the culture flask was tapped and examined under a microscope for detachment of the cells to the flask. If necessary, the cells were placed back into the incubator to wait for another 5-10 minutes until 85% of the cells were detached from each other.
2.6. 6-9 mL of complete cell culture medium was quickly added to terminate the trypsin reaction, and then the cells were transferred to a sterile 15 mL centrifuge tube. The cells were pelleted by centrifugation at 300 g for 5-7 minutes in a centrifuge, and then the supernatant was decanted.
2.7. The cells were resuspended in 10 mL of fresh cell culture medium and the cells were counted by using a hemocytometer.
2.8. 3,000 cells contained in 100 μL were inoculated into each well of a sterile 96-well cell culture plate (Corning 3903) and cultured overnight at 37° C. under 5% CO₂.

### 3. Dilution and Treatment of Compounds

3.1. All batches of compounds were dissolved in dimethyl sulfoxide (DMSO) to prepare a 20 mM stock.
3.2. 3 μL of 20 mM stock of each compound was added to 27 μL of DMSO and mixed thoroughly, and then diluted at a dilution ratio of 1:2 (20 μL compound+40 μL DMSO) until the 10-12 points ended. After the compounds were diluted, they were set aside for use.
3.3. The cell culture plate was taken out from the incubator, washed once with PBS, and replaced with a fresh culture medium free of serum. A mixture of TNF-α (100 ng/mL), SMAC mimic (SmacM, 100 nM) and Z_VAD-FMK (10 μM) was added to induce the programmed necrosis in HT-29 cells.
3.4. The compound dilution plate was prepared, 0.5 μL of the diluted compound was added to the corresponding 96-well plate, and mixed gently. Two duplicate wells were prepared for each compound.
3.5. The treated 96-well plate to which the compounds had been added was placed back into the 37° C. incubator containing 5% CO2 and incubated overnight.

### 4. Cell Viability Detection

The luminescence cell viability detection kit was used to calculate the activity of the compound by measuring the ATP content in viable cells.
4.1. The CellTiter-Glo® buffer and dry powdered substrate were equilibrated to room temperature before use.
4.2. The CellTiter-Glo® substrate was resuspended with Cell Titer-Glo® buffer, then homogeneously mixed by gently vortexing.
4.3. The cell culture plate was taken out and equilibrated at room temperature for 10 minutes (usually not suitable if more than 30 minutes).
4.4. 20 μL of enzyme/substrate mixture was added into the 96-well assay plate with a multi-channel pipettor.
4.5. The 96-well assay plate was placed on a shaker and the content was mixed for 2-3 minutes to promote the cell lysis.
4.6. The 96-well assay plate was incubated at room temperature for 10 minutes to stabilize the luminescence signal.
4.7. The multifunctional Microplate reader EnVision was used to detect the chemiluminescence signal of viable cells.
4.8 The survival rate of viable cells was calculated by detecting the chemiluminescence signal.

### 5. Data Processing

5.1. The average background value of each assay plate and the average value of the control group were calculated, and the uniformity of the assay data for the entire plate was calculated to determine the validity of the assay.
5.2. The average of the cell survival rates repeated twice according to Item 4.8 was taken for each compound at different concentration points.
5.3. The above calculated data were input into GraphPad 8.0 software, the concentration response curve of the compound was fitted to determine the EC₅₀ value of each compound.
5.4. The EC₅₀ curve of each compound was collected up in GraphPad 8.0 software, the data processed in GraphPad 8.0 software was exported, and a report on the activity of compounds was made.

The assay results are shown in Table 1.

TABLE 1
In vitro biological activity assay results of
RIP1 inhibitors of the present disclosure

| Compound No. | EC₅₀(nM) |
|---|---|
| Compound 1 | +++ |
| Compound 2 | +++ |
| Compound 3 | +++ |
| Compound 4 | +++ |
| Compound 5 | +++ |
| Compound 6 | +++ |
| Compound 7 | ++ |
| Compound 8 | ++ |
| Compound 9 | ++ |
| Compound 10 | + |
| Compound 11 | +++ |
| Compound 12 | + |
| Compound 13 | +++ |
| Compound 14 | +++ |
| Compound 15 | +++ |
| Compound 16 | +++ |
| Compound 17 | +++ |
| Compound 18 | +++ |
| Compound 19 | +++ |
| Compound 20 | +++ |
| Compound 21 | ++ |
| Compound 22 | +++ |
| Compound 23 | +++ |
| Compound 24 | +++ |
| Compound 25 | +++ |
| Compound 26 | +++ |
| Compound 27 | +++ |
| Compound 28 | +++ |
| Compound 29 | +++ |
| Compound 30 | + |
| Compound 31 | +++ |
| Compound 32 | +++ |
| Compound 33 | +++ |
| Compound 34 | +++ |
| Compound 35 | +++ |
| Compound 36 | +++ |
| Compound 37 | +++ |
| Compound 38 | +++ |
| Compound 39 | +++ |
| Compound 40 | +++ |
| Compound 41 | ++ |
| Compound 42 | +++ |
| Compound 43 | +++ |
| Compound 44 | +++ |
| Compound 45 | +++ |
| Compound 46 | +++ |
| Compound 47 | +++ |
| Compound 48 | +++ |
| Compound 49 | +++ |
| Compound 50 | +++ |
| Compound 51 | +++ |
| Compound 52 | +++ |
| Compound 53 | +++ |
| Compound 54 | +++ |
| Compound 55 | +++ |
| Compound 56 | + |
| Compound 57 | +++ |
| Compound 58 | +++ |
| Compound 59 | +++ |
| Compound 60 | + |
| Compound 61 | +++ |
| Compound 62 | +++ |
| Compound 63 | +++ |
| Compound 64 | +++ |
| Compound 65 | +++ |
| Compound 66 | +++ |

Note:
In the above table, + indicates that the EC₅₀ value of the corresponding compound is >1000 nM; ++ indicates that the EC₅₀ value of the corresponding compound is >100 nM and ≤1000 nM; and +++ indicates that the EC₅₀ value of the corresponding compound is ≤100 nM.

It can be seen that the above representative compounds of the present disclosure have excellent biological activity in inhibiting programmed necrosis of cells, and thus can be used to prevent or treat diseases mediated by RIP1 kinase.

### Example 68: Rapid Brain Distribution Assay

The contents in plasma and tissue samples were measured by LC-MS/MS to obtain the ratio of the concentration in the brain tissue sample to that in the plasma sample, and compounds with blood-brain penetration could be quickly screened.

### Assay Condition and Process:

### 1. Reagents

Chromatographically pure acetonitrile: purchased from Fisher Company, lot number 207866;

Chromatographically pure methanol: purchased from Fisher Company, lot number 211511;

Formic acid: purchased from Fisher Company, lot number 202674;

DMSO: purchased from Fisher Company, lot number 2167209;

Polyethylene glycol-15-hydroxystearate (HS): purchased from Sigma Company, lot number BCCB9630.

### 2. Instruments

AB SCIEX Triple Quad 5500 Plus liquid chromatography-mass spectrometer (AB SCIEX company's product, USA), including Triple Quad 5500 Plus triple quadrupole tandem mass spectrometer, equipped with an ESI source and Analyst 1.7.1 data processing system;

The liquid phase chromatography: AB SCIEX liquid phase chromatography, equipped with a high-pressure infusion pump, an automatic sampler, and a column oven;

1/100,000 analytical balance: Sartorius SQP (max. 30 g, d=0.01 mg);

Multichannel pipette ((10 μL, 120 μL, 300 μL)) purchased from Sartorius Biohit; monochannel pipette ((10 μL, 100 μL, 200 μL, 1000 μL)), purchased from Sartorius Biohit;

VOTREX-2 Genie Vortex shaker;

Eppendorf 5810R low-temperature high-speed centrifuge;

4° C. refrigerator, purchased from Thermo Company;

−20° C. and −80° C. refrigerators, purchased from Haier Company.

### 3. Compound Preparation

3.1. A group of compounds (usually 3-4 compounds, the molecular weight difference between each other was greater than 5), weighing 1-2 mg of each compound, were added into the same 4 mL weighing bottle.
3.2. A mixed solvent of 10% DMSO+20% polyethylene glycol-15-hydroxystearate+70% H₂O was used to prepare each group to a volume of 1.5 mL-2 mL.
3.3. The mixture was ultrasonically vortexed until a homogeneous solution was obtained for intraperitioneal administration.

### 4. Administration

4.1. Three CD1 mice were prepared for each group and weighed.
4.2. The administration volume was 10 mL/kg, and the mice were administered intraperitoneally.
4.3. Timing was started after administration, and plasma samples and brain tissue samples were taken one hour later.
4.4. The brain tissue sample was weighed, and then 4 fold volume of physiological saline was added. The resulting mixture was homogenized, and frozen at −20° C. until analysis.

### 5. LC-MS/MS Method

5.1. The exact molecular weight of a compound was found from the compound information table.
5.2. The corresponding parent ions and fragment ions were found, and the mass spectrometry condition and the liquid phase (chromatography) method were optimized to find the appropriate condition and method.

### 6. Sample Analysis Process

6.1. Plasma sample (5-fold dilution): 10 μL of the plasma sample was taken, and 40 μL of the blank plasma sample and 50 μL of the corresponding blank tissue sample were added.
6.2 Tissue sample processing: 50 μL of the tissue sample (brain, kidney, liver and the like) was taken, and 50 μL of the blank plasma sample was added.
6.3. The above tissue sample and the diluted plasma sample were taken, and 500 μL of the internal standard solution was added to precipitate the protein. The resulting mixture was vortexed for 2 minutes, stored by freezing at −20° C. for 1 hour and centrifuged at 3800 rpm for 15 minutes at 4° C. The supernatant was taken, 5-fold diluted with 50% MeOH, and then vortexed for 2 minutes, ready for measurement.

### 7. Sample Detection

The sample was detected by using an appropriate LC-MS/MS method.

### 8. Data Processing

Distribution ratio of tissue/plasma=(peak area of compound in tissue/peak area of internal standard)/(peak area of compound in plasma/peak area of internal standard)

TABLE 2
Rapid brain distribution assay results of
RIP1 inhibitors of the present disclosure

| Compound No. | B/P |
|---|---|
| Compound 1 | 0.735 |
| Compound 2 | 0.62 |
| Compound 3 | 3.96 |
| Compound 4 | 1.44 |
| Compound 5 | 0.75 |
| Compound 6 | 1.24 |
| Compound 7 | 0.12 |
| Compound 9 | 2.85 |
| Compound 10 | 1.34 |
| Compound 11 | 1.23 |
| Compound 14 | 2.92 |
| Compound 15 | 0.406 |
| Compound 16 | 2.24 |
| Compound 24 | 1.33 |
| Compound 28 | 1.91 |
| Compound 30 | 0.429 |
| Compound 31 | 2.38 |
| Compound 32 | 1.27 |
| Compound 33 | 0.563 |
| Compound 36 | 3.62 |
| Compound 37 | 1.06 |
| Compound 38 | 2.46 |
| Compound 39 | 2.07 |
| Compound 40 | 1.61 |
| Compound 42 | 2.45 |
| Compound 44 | 3.25 |
| Compound 45 | 2.28 |
| Compound 51 | 2.46 |
| Compound 52 | 1.1 |
| Compound 54 | 1.44 |
| Compound 55 | 2.61 |
| Compound 56 | 1.2 |
| Compound 57 | 2.16 |
| Compound 58 | 2.93 |
| Compound 61 | 1.17 |
| Compound 62 | 0.388 |
| Compound 63 | 3.77 |
| Compound 65 | 1.12 |
| Compound 66 | 3.62 |

It can be seen that the above representative compounds of the present disclosure had excellent blood-brain penetration and could enter the central nervous system through the blood-brain barrier to prevent or treat RIP1 kinase-mediated diseases located in the central nervous system.

The above embodiments and examples are provided so that those skilled in the art can more clearly understand the essence and effects of the present disclosure. However, these embodiments and examples are for illustrative purposes only and in no way limit the claims of the present disclosure.

All publications cited in the specification are incorporated by reference in their entirety. It will be apparent to those of ordinary skill in the art that certain changes and modifications can be made in the embodiments and examples of the present disclosure without departing from the spirit or scope of the appended claims.

1. A compound of general formula (I), or a pharmaceutically acceptable salt, hydrate, solvate and stereoisomer thereof: 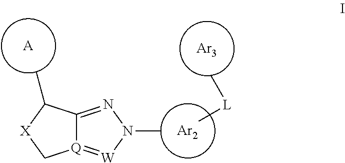
wherein,
Ring A is C₆-C₁₂aryl; C₃-C₈cycloalkyl; or 5 to 12 membered heteroaryl containing 1 or 2 heteroatoms independently selected from N, O and S, wherein said aryl, cycloalkyl or heteroaryl is substituted with 0, 1, 2 or 3 substituents independently selected from halogen, C₁-C₆alkyl, haloC₁-C₆alkyl, C₂-C₆alkenyl, C₂-C₆alkynyl, C₁-C₆alkoxy, haloC₁-C₆alkoxy, cyano, hydroxy, nitro, —NR¹R², —C(═O)R¹, —C(═O)OR¹, —OC(═O)R¹, —C(═O)NR¹R², —SR¹, —S(═O)R¹, —S(═O)₂R¹ and —S(═O)₂NR¹R²;
Ring Ar₂ is C₆-C₁₂arylene; C₃-C₈cycloalkylene; 3 to 12 membered heterocyclylene containing 1 or 2 heteroatoms independently selected from N, O and S; or 5 to 12 membered heteroarylene containing 1 or 2 heteroatoms independently selected from N, O and S, wherein said arylene, cycloalkylene, heterocyclylene or heteroarylene is substituted with 0, 1, 2 or 3 substituents independently selected from halogen, C₁-C₆alkyl, haloC₁-C₆alkyl, C₂-C₆alkenyl, C₂-C₆alkynyl, C₁-C₆alkoxy, haloC₁-C₆alkoxy, cyano, hydroxy, nitro, —NR¹R², —C(═O)R¹, —C(═O)OR¹, —OC(═O)R¹, —C(═O)NR¹R², —SR¹, —S(═O)R¹, —S(═O)₂R¹ and —S(═O)₂NR¹R², and wherein said heterocyclylene is optionally further substituted with one ═O group;
Ring Ar₃ is C₆-C₁₂aryl; 3 to 12 membered heterocyclyl containing 1 or 2 heteroatoms independently selected from N, O and S; or 5 to 12 membered heteroaryl containing 1 or 2 heteroatoms independently selected from N, O and S, wherein said aryl, heterocyclyl or heteroaryl is substituted with 0, 1, 2 or 3 substituents independently selected from halogen, C₁-C₆alkyl, haloC₁-C₆alkyl, C₂-C₆alkenyl, C₂-C₆alkynyl, C₁-C₆alkoxy, haloC₁-C₆alkoxy, cyano, hydroxy, nitro, —NR¹R², —C(═O)R¹, —C(═O)OR¹, —OC(═O)R¹, —C(═O)NR¹R², —SR¹, —S(═O)R¹, —S(═O)₂R¹ and —S(═O)₂NR¹R², and wherein said heterocyclyl is optionally further substituted with one ═O group;
L is selected from C₁-C₆alkylene, C₂-C₆alkenylene or C₂-C₆alkynylene;
Xis CR¹R², O orS;
Q is C or N;
W is CR³, —C(═O)— or N;
the dashed line between Q and W represents a single bond or a double bond, provided that when Q is C, the dashed line between Q and W represents a double bond and at the same time W is CR³ or N, and when Q is N, the dashed line between Q and W represents a single bond and at the same time W is —C(═O)—;
each of R¹ and R² is independently H or C₁-C₆alkyl; and
R³ is H, halogen, C₁-C₆alkyl, haloC₁-C₆alkyl, C₁-C₆alkoxy, haloC₁-C₆alkoxy, cyano, hydroxy, nitro or —NR¹R².

2. The compound according to claim 1 or a pharmaceutically acceptable salt, hydrate, solvate and stereoisomer thereof, wherein Ring A is C₆-C₁₂aryl, preferably phenyl or naphthyl, more preferably phenyl, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy, preferably substituted with 0, 1 or 2 substituents independently selected from halogen and cyano, more preferably substituted with 0, 1 or 2 substituents independently selected from F, Cl and cyano.

3. A compound according to any one of the preceding claims, or a pharmaceutically acceptable salt, hydrate, solvate and stereoisomer thereof, wherein Ring A is C₃-C₈ cycloalkyl, preferably C₄-C₇ cycloalkyl, more preferably cyclopentyl or cyclohexyl, still more preferably cyclohexyl, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy, preferably substituted with 0, 1 or 2 substituents independently selected from halogen and cyano, more preferably substituted with 0, 1 or 2 substituents independently selected from F, Cl and cyano.

4. A compound according to any one of the preceding claims, or a pharmaceutically acceptable salt, hydrate, solvate and stereoisomer thereof, wherein Ring A is 5 to 12 membered heteroaryl containing 1 or 2 heteroatoms independently selected from N, O and S, preferably 5 or 6 membered heteroaryl containing 1 or 2 heteroatoms independently selected from N, O and S, for example pyrrolyl, furanyl, thiophenyl, pyrazolyl, imidazolyl, oxazolyl, isoxazolyl, thiazolyl, isothiazolyl, oxadiazolyl, thiadiazolyl, tetrazolyl, triazolyl, pyridinyl, pyridazinyl, pyrimidinyl, pyrazinyl or triazinyl, more preferably 6 membered heteroaryl containing 1 or 2 N atoms, for example pyridinyl, pyridazinyl, pyrimidinyl or pyrazinyl, still more preferably pyridinyl, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy, preferably substituted with 0, 1 or 2 substituents independently selected from halogen and cyano, more preferably substituted with 0, 1 or 2 substituents independently selected from F, Cl and cyano.

5. A compound according to any one of the preceding claims, or a pharmaceutically acceptable salt, hydrate, solvate and stereoisomer thereof, wherein Ring Ar₂ is C₆-C₁₂ arylene, preferably phenylene or naphthylene, more preferably phenylene, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy, preferably substituted with 0 or 1 C₁-C₆alkyl groups, more preferably substituted with 0 or 1 substituents independently selected from methyl and ethyl.

6. A compound according to any one of the preceding claims, or a pharmaceutically acceptable salt, hydrate, solvate and stereoisomer thereof, wherein Ring Ar₂ is C₃-C₈ cycloalkylene, preferably C₄-C₇ cycloalkylene, more preferably cyclopentylene or cyclohexylene, still more preferably cyclopentylene, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy, preferably substituted with 0 or 1 C₁-C₆alkyl groups, more preferably substituted with 0 or 1 substituents independently selected from methyl and ethyl.

7. A compound according to any one of the preceding claims, or a pharmaceutically acceptable salt, hydrate, solvate and stereoisomer thereof, wherein Ring Ar₂ is 3 to 12 membered heterocyclylene containing 1 or 2 heteroatoms independently selected from N, O and S, preferably 5 or 6 membered heterocyclylene containing 1 or 2 heteroatoms independently selected from N, O and S, for example pyrrolidinylene, tetrahydrofuranylene, tetrahydrothiophenylene, pyrazolidinylene, imidazolidinylene, oxazolidinylene, isoxazolidinylene, thiazolidinylene, isothiazolidinylene, pyrrolinylene, dihydrofuranylene, dihydrothiophenylene, pyrazolinylene, imidazolinylene, oxazolinylene, isoxazolinylene, thiazolinylene, isothiazolinylene, piperidinylene, piperazinylene, morpholinylene, thiomorpholinylene, dihydropyridinylene, dihydropyrazinylene, dihydropyrimidinylene, dihydropyridazinylene, more preferably 6 membered heterocyclylene containing 1 N atom, for example piperidinylene, dihydropyridinylene, still more preferably dihydropyridinylene, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy and further substituted with one ═O group, preferably substituted with 0 or 1 C₁-C₆alkyl groups and further substituted with one ═O group, more preferably substituted with 0 or 1 substituents independently selected from methyl and ethyl and further substituted with one ═O group.

8. A compound according to any one of the preceding claims, or a pharmaceutically acceptable salt, hydrate, solvate and stereoisomer thereof, wherein Ring Ar₂ is 5 to 12 membered heteroarylene containing 1 or 2 heteroatoms independently selected from N, O and S, preferably 5 or 6 membered heteroarylene containing 1 or 2 heteroatoms independently selected from N, O and S, for example pyrrolylene, furanylene, thiophenylene, pyrazolylene, imidazolylene, oxazolylene, isoxazolylene, thiazolylene, isothiazolylene, oxadiazolylene, thiadiazolylene, tetrazolylene, triazolylene, pyridinylene, pyridazinylene, pyrimidinylene, pyrazinylene or triazinylene, more preferably 6 membered heteroarylene containing 1 or 2 N atoms, for example pyridinylene, pyridazinylene, pyrimidinylene or pyrazinylene, still more preferably pyridinylene or pyrimidinylene, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy, preferably substituted with 0 or 1 C₁-C₆alkyl groups, more preferably substituted with 0 or 1 substituents independently selected from methyl and ethyl.

9. A compound according to any one of the preceding claims, or a pharmaceutically acceptable salt, hydrate, solvate and stereoisomer thereof, wherein Ring Ar₃ is C₆-C₁₂aryl, preferably phenyl or naphthyl, more preferably phenyl, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano, hydroxy, —NR¹R² and —C(═O)NR¹R², preferably substituted with 0, 1 or 2 substituents independently selected from halogen, C₁-C₆alkyl, C₁-C₆alkoxy, hydroxy, —NH₂ and —C(═O)NH₂, more preferably substituted with 0, 1 or 2 substituents independently selected from chloro, methyl, methoxy, hydroxy, —NH₂ and —C(═O)NH₂.

10. A compound according to any one of the preceding claims, or a pharmaceutically acceptable salt, hydrate, solvate and stereoisomer thereof, wherein Ring Ar₃ is 3 to 12 membered heterocyclyl containing 1 or 2 heteroatoms independently selected from N, O and S, preferably 5 or 6 membered heterocyclyl containing 1 or 2 heteroatoms independently selected from N, O and S, for example pyrrolidinyl, tetrahydrofuranyl, tetrahydrothiophenyl, pyrazolidinyl, imidazolidinyl, oxazolidinyl, isoxazolidinyl, thiazolidinyl, isothiazolidinyl, pyrrolinyl, dihydrofuranyl, dihydrothiophenyl, pyrazolinyl, imidazolinyl, oxazolinyl, isoxazolinyl, thiazolinyl, isothiazolinyl, piperidinyl, piperazinyl, morpholinyl, thiomorpholinyl, dihydropyridinyl, dihydropyrazinyl, dihydropyrimidinyl, dihydropyridazinyl, more preferably 6 membered heterocyclyl containing 1 or 2 N atoms, for example dihydropyridinyl, dihydropyrazinyl, dihydropyrimidinyl, dihydropyridazinyl, still more preferably dihydropyrimidinyl, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano, hydroxy, —NR¹R² and —C(═O)NR¹R² and further substituted with one ═O group, preferably substituted with 0, 1 or 2 substituents independently selected from halogen, C₁-C₆alkyl, C₁-C₆alkoxy, hydroxy, —NH₂ and —C(═O)NH₂ and further substituted with one ═O group, more preferably substituted with 0, 1 or 2 substituents independently selected from chloro, methyl, methoxy, hydroxy, —NH₂ and —C(═O)NH₂ and further substituted with one ═O group.

11. A compound according to any one of the preceding claims, or a pharmaceutically acceptable salt, hydrate, solvate and stereoisomer thereof, wherein Ring Ar₃ is 5 to 12 membered heteroaryl containing 1 or 2 heteroatoms independently selected from N, O and S, for example pyrrolyl, furanyl, thiophenyl, pyrazolyl, imidazolyl, oxazolyl, isoxazolyl, thiazolyl, isothiazolyl, oxadiazolyl, thiadiazolyl, tetrazolyl, triazolyl, pyridinyl, pyridazinyl, pyrimidinyl, pyrazinyl, triazinyl, benzimidazolyl, benzofuranyl, benzothiophenyl, benzoxadiazolyl, benzothiadiazolyl, benzothiazolyl, imidazopyridinyl, imidazopyrimidinyl, imidazopyridazinyl, cinnolinyl, furopyridinyl, indazolyl, indolyl, isoindolyl, isoquinolinyl, naphthyridinyl, purinyl, quinolinyl, thienopyridinyl, preferably pyrazolyl, imidazolyl, thiazolyl, pyridinyl, pyridazinyl, pyrimidinyl, pyrazinyl, imidazopyridinyl or imidazopyridazinyl, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano, hydroxy, —NR¹R² and —C(═O)NR¹R², preferably substituted with 0, 1 or 2 substituents independently selected from halogen, C₁-C₆alkyl, C₁-C₆alkoxy, hydroxy, —NH₂ and —C(═O)NH₂, more preferably substituted with 0, 1 or 2 substituents independently selected from chloro, methyl, methoxy, hydroxy, —NH₂ and —C(═O)NH₂.

12. A compound according to any one of the preceding claims, or a pharmaceutically acceptable salt, hydrate, solvate and stereoisomer thereof, wherein L is C₁-C₆alkylene or C₂-C₆alkynylene, preferably methylene, ethylene, n-propylene or ethynylene, more preferably ethylene or ethynylene.

13. A compound according to any one of the preceding claims, or a pharmaceutically acceptable salt, hydrate, solvate and stereoisomer thereof, wherein X is CR¹R² or O, preferably CH₂ or O.

14. A compound according to any one of the preceding claims, or a pharmaceutically acceptable salt, hydrate, solvate and stereoisomer thereof, wherein Q is C and the dashed line between Q and W represents a double bond and at the same time W is CR³ or N.

15. A compound according to any one of the preceding claims, or a pharmaceutically acceptable salt, hydrate, solvate and stereoisomer thereof, wherein Q is N and the dashed line between Q and W represents a single bond and at the same time W is —C(═O)—.

16. A compound according to any one of the preceding claims, or a pharmaceutically acceptable salt, hydrate, solvate and stereoisomer thereof, wherein W is CR³, preferably CH, CF, CCl, CBr, C(CH₃) or C(C₂H₅).

17. A compound according to any one of the preceding claims, or a pharmaceutically acceptable salt, hydrate, solvate and stereoisomer thereof, wherein W is N.

18. A compound according to any one of the preceding claims, or a pharmaceutically acceptable salt, hydrate, solvate and stereoisomer thereof, wherein each of R¹ and R² is independently H.

19. A compound according to any one of the preceding claims, or a pharmaceutically acceptable salt, hydrate, solvate and stereoisomer thereof, wherein each of R¹ and R² is independently C₁-C₆alkyl, preferably methyl or ethyl.

20. A compound according to any one of the preceding claims, or a pharmaceutically acceptable salt, hydrate, solvate and stereoisomer thereof, wherein R³ is H, halogen or C₁-C₆alkyl, preferably H, F, Cl, Br, methyl or ethyl, more preferably H, Cl or methyl.

21. A compound according to any one of the preceding claims, or a pharmaceutically acceptable salt, hydrate, solvate and stereoisomer thereof, wherein
Ring A is phenyl or naphthyl; C₄-C₇ cycloalkyl; or 5 or 6 membered heteroaryl containing 1 or 2 heteroatoms independently selected from N, O and S, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy;
Ring Ar₂ is phenylene or naphthylene; C₄-C₇cycloalkylene; 5 or 6 membered heterocyclylene containing 1 or 2 heteroatoms independently selected from N, O and S; or 5 or 6 membered heteroarylene containing 1 or 2 heteroatoms independently selected from N, O and S, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano and hydroxy and the heterocyclylene is further substituted with one ═O group;
Ring Ar₃ is phenyl or naphthyl; 5 or 6 membered heterocyclyl containing 1 or 2 heteroatoms independently selected from N, O and S; or 5 to 12 membered heteroaryl containing 1 or 2 heteroatoms independently selected from N, O and S, wherein the above groups are substituted with 0, 1 or 2 substituents independently selected from halogen, C₁-C₆alkyl, haloC₁-C₆alkyl, C₁-C₆alkoxy, haloC₁-C₆alkoxy, cyano, hydroxy, —NR¹R² and —C(═O)NR¹R² and the heterocyclyl is further substituted with one ═O group;
L is selected from C₁-C₆alkylene or C₂-C₆alkynylene;
X is CR¹R² or O;
Q is C or N;
W is CR³, —C(═O)— or N;
the dashed line between Q and W represents a single bond or a double bond, provided that when Q is C, the dashed line between Q and W represents a double bond and at the same time W is CR³ or N, and when Q is N, the dashed line between Q and W represents a single bond and at the same time W is —C(═O)—;
each of R¹ and R² is independently H, methyl or ethyl; and
R³ is H, halogen or C₁-C₆alkyl.

22. A compound according to any one of the preceding claims, or a pharmaceutically acceptable salt, hydrate, solvate and stereoisomer thereof, wherein
Ring A is phenyl; cyclopentyl or cyclohexyl; or 6 membered heteroaryl containing 1 or 2 N atoms, wherein the above groups are substituted with 0, 1 or 2 substituents independently selected from halogen and cyano;
Ring Ar₂ is phenylene; cyclopentylene or cyclohexylene; 6 membered heterocyclylene containing 1 N atom; or 6 membered heteroarylene containing 1 or 2 N atoms, wherein the above groups are substituted with 0 or 1 C₁-C₆alkyl groups and the heterocyclylene is further substituted with one ═O group;
Ring Ar₃ is phenyl; 6 membered heterocyclyl containing 1 or 2 N atoms; or pyrazolyl, imidazolyl, thiazolyl, pyridinyl, pyridazinyl, pyrimidinyl, pyrazinyl, imidazopyridinyl or imidazopyridazinyl, wherein the above groups are substituted with 0, 1 or 2 substituents independently selected from halogen, C₁-C₆alkyl, C₁-C₆alkoxy, hydroxy, —NH₂ and —C(═O)NH₂ and the heterocyclyl is further substituted with one ═O group;
L is selected from methylene, ethylene, n-propylene or ethynylene;
X is CH₂ or O;
Q is C or N;
W is CR³, —C(═O)— or N;
the dashed line between Q and W represents a single bond or a double bond, provided that when Q is C, the dashed line between Q and W represents a double bond and at the same time W is CR³ or N, and when Q is N, the dashed line between Q and W represents a single bond and at the same time W is —C(═O)—; and
R³ is H, F, Cl, Br, methyl or ethyl.

23. A compound according to any one of the preceding claims, or a pharmaceutically acceptable salt, hydrate, solvate and stereoisomer thereof, wherein
Ring A is phenyl; cyclohexyl; or pyridinyl, wherein the above groups are substituted with 0, 1 or 2 substituents independently selected from F, Cl and cyano;
Ring Ar₂ is phenylene; cyclopentylene; dihydropyridinylene; or pyridinylene or pyrimidinylene, wherein the above groups are substituted with 0 or 1 substituents independently selected from methyl and ethyl and the dihydropyridinylene is further substituted with one ═O group;
Ring Ar₃ is phenyl; dihydropyrimidinyl; or pyrazolyl, imidazolyl, thiazolyl, pyridinyl, pyridazinyl, pyrimidinyl, pyrazinyl, imidazopyridinyl or imidazopyridazinyl, wherein the above groups are substituted with 0, 1 or 2 substituents independently selected from chloro, methyl, methoxy, hydroxy, —NH₂ and —C(═O)NH₂ and the dihydropyrimidinyl is further substituted with one ═O group;
L is selected from ethylene or ethynylene;
X is CH₂ or O;
Q is C or N;
W is CR³, —C(═O)— or N;
the dashed line between Q and W represents a single bond or a double bond, provided that when Q is C, the dashed line between Q and W represents a double bond and at the same time W is CR³ or N, and when Q is N, the dashed line between Q and W represents a single bond and at the same time W is —C(═O)—; and
R³ is H, Cl or methyl.

24. A compound or a pharmaceutically acceptable salt, hydrate, solvate and stereoisomer thereof, wherein the compound is any one selected from Compounds 1-66 depicted in the following table:

| | |
|---|---|
| 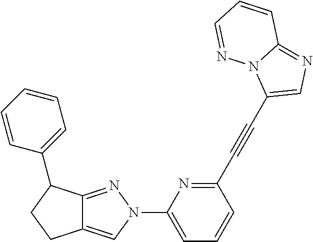 [SMILES: C(#Cc1cnc2cccnn12)c1cccc(-n2cc3c(n2)C(c2ccccc2)CC3)n1] | Compound 1 |
| 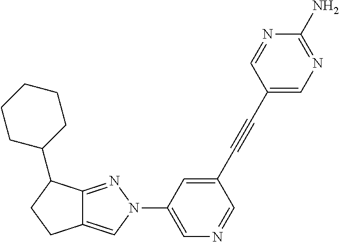 [SMILES: Nc1ncc(C#Cc2cncc(-n3cc4c(n3)C(C3CCCCC3)CC4)c2)cn1] | Compound 2 |
| 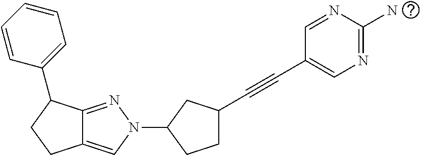 [SMILES: Nc1ncc(C#CC2CCC(n3cc4c(n3)C(c3ccccc3)CC4)C2)cn1] | Compound 3 |
| 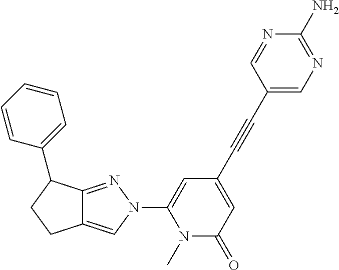 [SMILES: Cn1c(-n2cc3c(n2)C(c2ccccc2)CC3)cc(C#Cc2cnc(N)nc2)cc1=O] | Compound 4 |
| 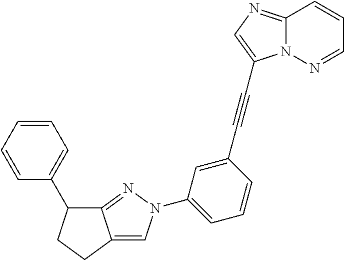 [SMILES: C(#Cc1cnc2cccnn12)c1cccc(-n2cc3c(n2)C(c2ccccc2)CC3)c1] | Compound 5 |
| 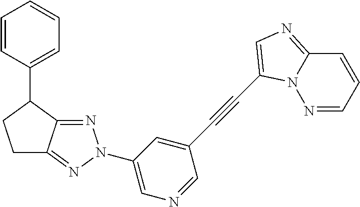 [SMILES: C(#Cc1cnc2cccnn12)c1cncc(-n2nc3c(n2)C(c2ccccc2)CC3)c1] | Compound 6 |
| 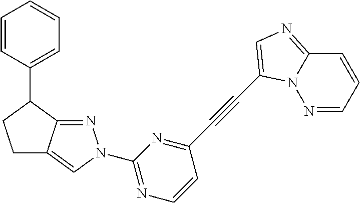 [SMILES: C(#Cc1cnc2cccnn12)c1ccnc(-n2cc3c(n2)C(c2ccccc2)CC3)n1] | Compound 7 |
| 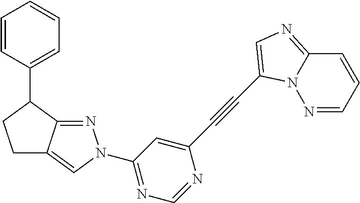 [SMILES: C(#Cc1cnc2cccnn12)c1cc(-n2cc3c(n2)C(c2ccccc2)CC3)ncn1] | Compound 8 |
| 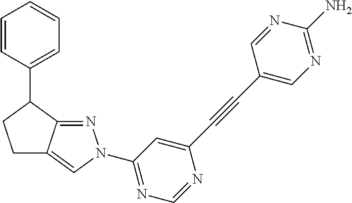 [SMILES: Nc1ncc(C#Cc2cc(-n3cc4c(n3)C(c3ccccc3)CC4)ncn2)cn1] | Compound 9 |
| 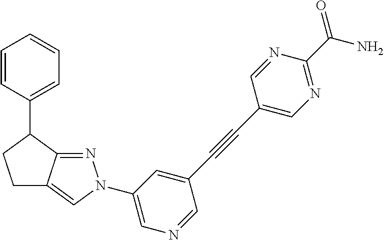 [SMILES: NC(=O)c1ncc(C#Cc2cncc(-n3cc4c(n3)C(c3ccccc3)CC4)c2)cn1] | Compound 10 |
| 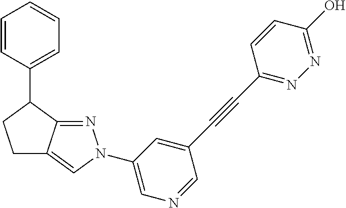 [SMILES: Oc1ccc(C#Cc2cncc(-n3cc4c(n3)C(c3ccccc3)CC4)c2)nn1] | Compound 11 |
| 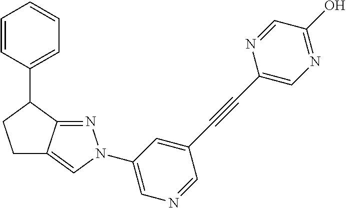 [SMILES: Oc1cnc(C#Cc2cncc(-n3cc4c(n3)C(c3ccccc3)CC4)c2)cn1] | Compound 12 |
| 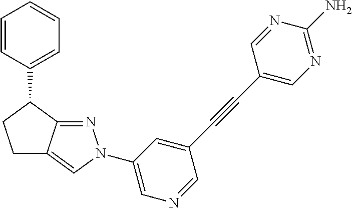 [SMILES: Nc1ncc(C#Cc2cncc(-n3cc4c(n3)[C@@H](c3ccccc3)CC4)c2)cn1] | Compound 13 |
| 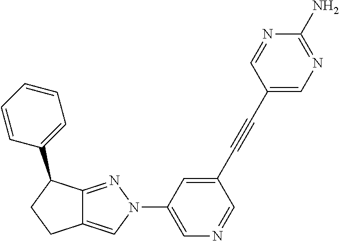 [SMILES: Nc1ncc(C#Cc2cncc(-n3cc4c(n3)[C@H](c3ccccc3)CC4)c2)cn1] | Compound 14 |
| 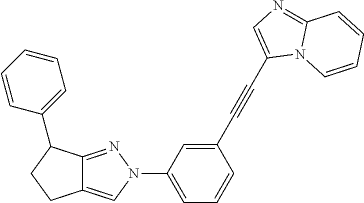 [SMILES: C(#Cc1cnc2ccccn12)c1cccc(-n2cc3c(n2)C(c2ccccc2)CC3)c1] | Compound 15 |
| 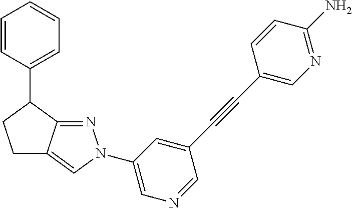 [SMILES: Nc1ccc(C#Cc2cncc(-n3cc4c(n3)C(c3ccccc3)CC4)c2)cn1] | Compound 16 |
| 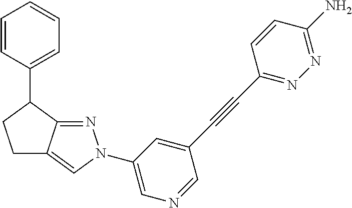 [SMILES: Nc1ccc(C#Cc2cncc(-n3cc4c(n3)C(c3ccccc3)CC4)c2)nn1] | Compound 17 |
| 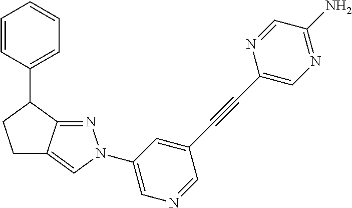 [SMILES: Nc1cnc(C#Cc2cncc(-n3cc4c(n3)C(c3ccccc3)CC4)c2)cn1] | Compound 18 |
| 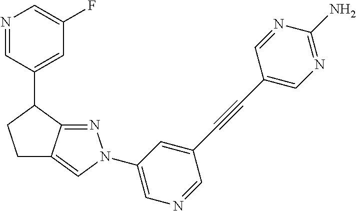 [SMILES: Nc1ncc(C#Cc2cncc(-n3cc4c(n3)C(c3cncc(F)c3)CC4)c2)cn1] | Compound 19 |
| 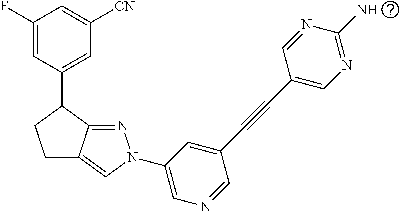 [SMILES: N#Cc1cc(F)cc(C2CCc3cn(-c4cncc(C#Cc5cnc(N)nc5)c4)nc32)c1] | Compound 20 |
| 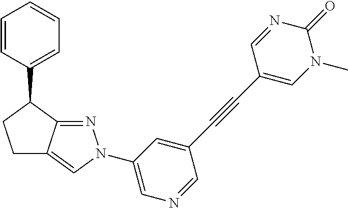 [SMILES: Cn1cc(C#Cc2cncc(-n3cc4c(n3)[C@H](c3ccccc3)CC4)c2)cnc1=O] | Compound 21 |
| 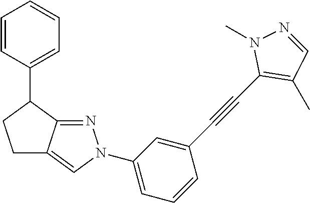 [SMILES: Cc1cnn(C)c1C#Cc1cccc(-n2cc3c(n2)C(c2ccccc2)CC3)c1] | Compound 22 |
| 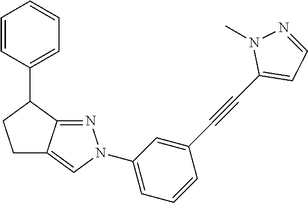 [SMILES: Cn1nccc1C#Cc1cccc(-n2cc3c(n2)C(c2ccccc2)CC3)c1] | Compound 23 |
| 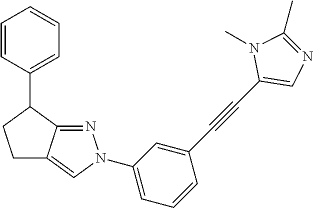 [SMILES: Cc1ncc(C#Cc2cccc(-n3cc4c(n3)C(c3ccccc3)CC4)c2)n1C] | Compound 24 |
| 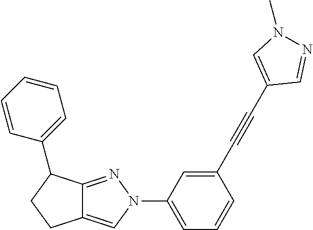 [SMILES: Cn1cc(C#Cc2cccc(-n3cc4c(n3)C(c3ccccc3)CC4)c2)cn1] | Compound 25 |
| 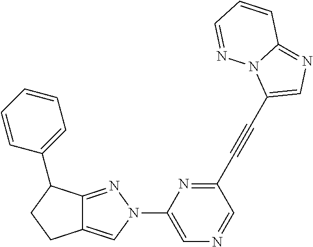 [SMILES: C(#Cc1cnc2cccnn12)c1cncc(-n2cc3c(n2)C(c2ccccc2)CC3)n1] | Compound 26 |
| 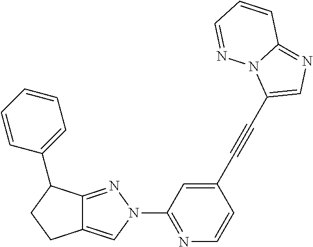 [SMILES: C(#Cc1cnc2cccnn12)c1ccnc(-n2cc3c(n2)C(c2ccccc2)CC3)c1] | Compound 27 |
| 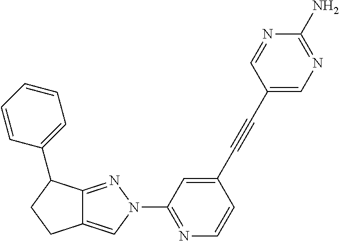 [SMILES: Nc1ncc(C#Cc2ccnc(-n3cc4c(n3)C(c3ccccc3)CC4)c2)cn1] | Compound 28 |
| 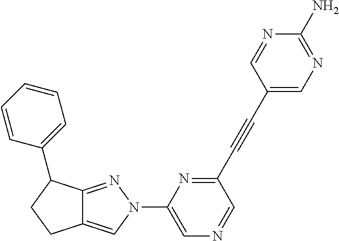 [SMILES: Nc1ncc(C#Cc2cncc(-n3cc4c(n3)C(c3ccccc3)CC4)n2)cn1] | Compound 29 |
| 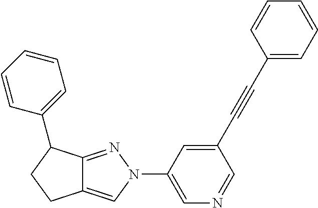 [SMILES: C(#Cc1cncc(-n2cc3c(n2)C(c2ccccc2)CC3)c1)c1ccccc1] | Compound 30 |
| 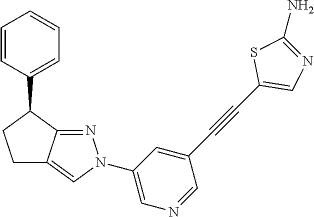 [SMILES: Nc1ncc(C#Cc2cncc(-n3cc4c(n3)[C@H](c3ccccc3)CC4)c2)s1] | Compound 31 |
| 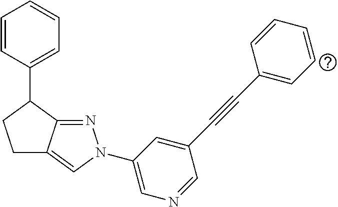 [SMILES: C(#Cc1cncc(-n2cc3c(n2)C(c2ccccc2)CC3)c1)c1ccccc1] | Compound 32 |
| 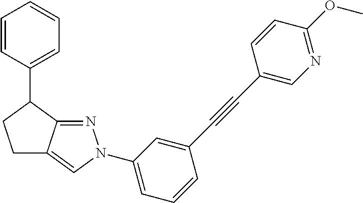 [SMILES: COc1ccc(C#Cc2cccc(-n3cc4c(n3)C(c3ccccc3)CC4)c2)cn1] | Compound 33 |
| 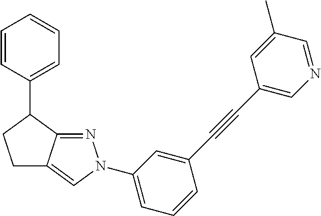 [SMILES: Cc1cncc(C#Cc2cccc(-n3cc4c(n3)C(c3ccccc3)CC4)c2)c1] | Compound 34 |
| 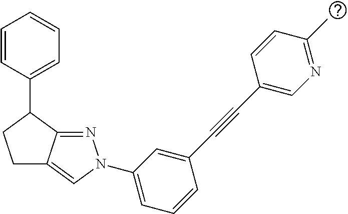 [SMILES: Cc1ccc(C#Cc2cccc(-n3cc4c(n3)C(c3ccccc3)CC4)c2)cn1] | Compound 35 |
| 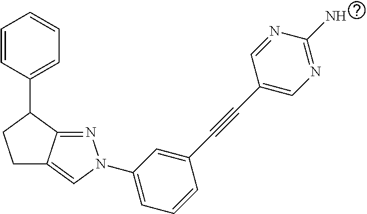 [SMILES: Nc1ncc(C#Cc2cccc(-n3cc4c(n3)C(c3ccccc3)CC4)c2)cn1] | Compound 36 |
| 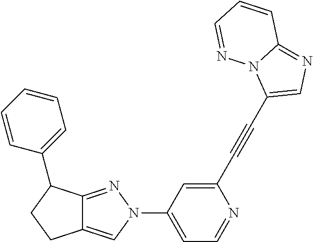 [SMILES: C(#Cc1cnc2cccnn12)c1cc(-n2cc3c(n2)C(c2ccccc2)CC3)ccn1] | Compound 37 |
| 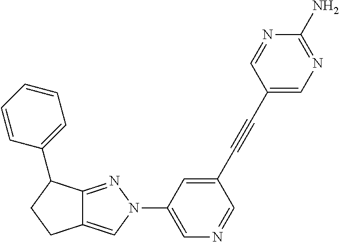 [SMILES: Nc1ncc(C#Cc2cncc(-n3cc4c(n3)C(c3ccccc3)CC4)c2)cn1] | Compound 38 |
| 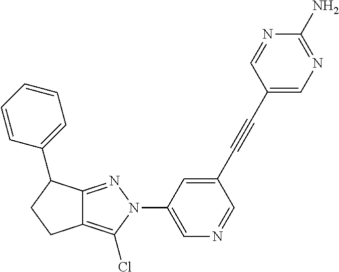 [SMILES: Nc1ncc(C#Cc2cncc(-n3nc4c(c3Cl)CCC4c3ccccc3)c2)cn1] | Compound 39 |
| 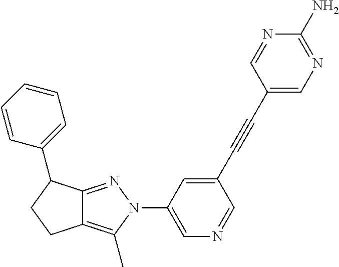 [SMILES: Cc1c2c(nn1-c1cncc(C#Cc3cnc(N)nc3)c1)C(c1ccccc1)CC2] | Compound 40 |
| 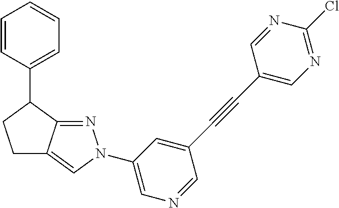 [SMILES: Clc1ncc(C#Cc2cncc(-n3cc4c(n3)C(c3ccccc3)CC4)c2)cn1] | Compound 41 |
| 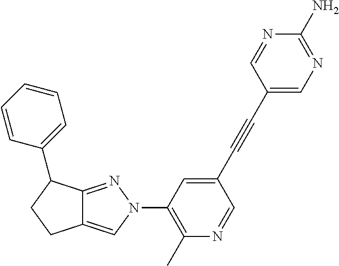 [SMILES: Cc1ncc(C#Cc2cnc(N)nc2)cc1-n1cc2c(n1)C(c1ccccc1)CC2] | Compound 42 |
| 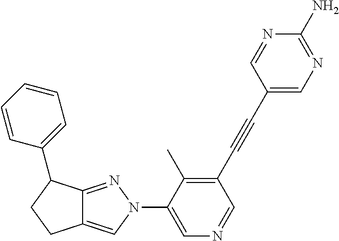 [SMILES: Cc1c(C#Cc2cnc(N)nc2)cncc1-n1cc2c(n1)C(c1ccccc1)CC2] | Compound 43 |
| 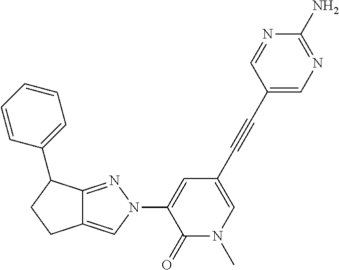 [SMILES: Cn1cc(C#Cc2cnc(N)nc2)cc(-n2cc3c(n2)C(c2ccccc2)CC3)c1=O] | Compound 44 |
| 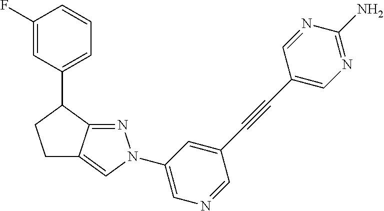 [SMILES: Nc1ncc(C#Cc2cncc(-n3cc4c(n3)C(c3cccc(F)c3)CC4)c2)cn1] | Compound 45 |
| 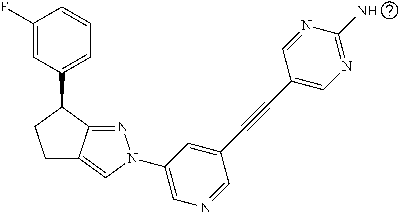 [SMILES: Nc1ncc(C#Cc2cncc(-n3cc4c(n3)[C@H](c3cccc(F)c3)CC4)c2)cn1] | Compound 46 |
| 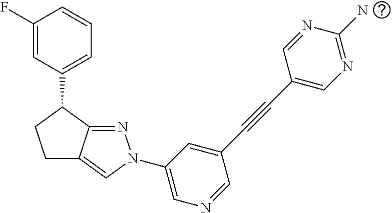 [SMILES: Nc1ncc(C#Cc2cncc(-n3cc4c(n3)[C@@H](c3cccc(F)c3)CC4)c2)cn1] | Compound 47 |
| 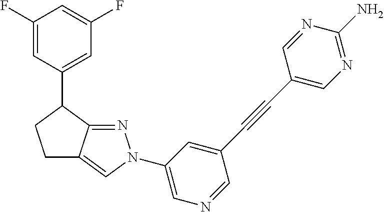 [SMILES: Nc1ncc(C#Cc2cncc(-n3cc4c(n3)C(c3cc(F)cc(F)c3)CC4)c2)cn1] | Compound 48 |
| 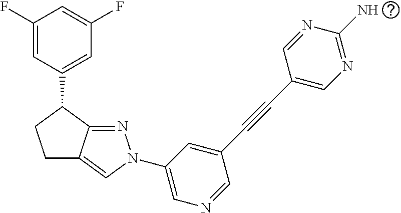 [SMILES: Nc1ncc(C#Cc2cncc(-n3cc4c(n3)[C@@H](c3cc(F)cc(F)c3)CC4)c2)cn1] | Compound 49 |
| 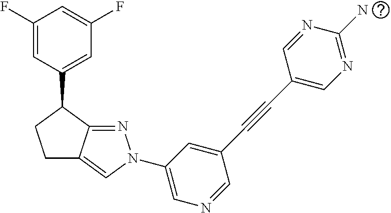 [SMILES: Nc1ncc(C#Cc2cncc(-n3cc4c(n3)[C@H](c3cc(F)cc(F)c3)CC4)c2)cn1] | Compound 50 |
| 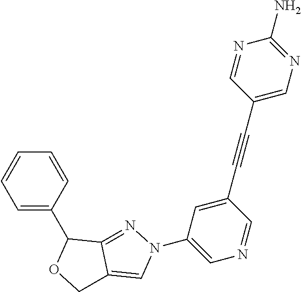 [SMILES: Nc1ncc(C#Cc2cncc(-n3cc4c(n3)C(c3ccccc3)OC4)c2)cn1] | Compound 51 |
| 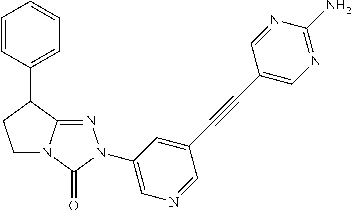 [SMILES: Nc1ncc(C#Cc2cncc(-n3nc4n(c3=O)CCC4c3ccccc3)c2)cn1] | Compound 52 |
| 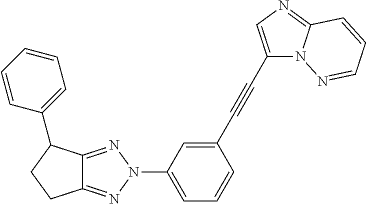 [SMILES: C(#Cc1cnc2cccnn12)c1cccc(-n2nc3c(n2)C(c2ccccc2)CC3)c1] | Compound 53 |
| 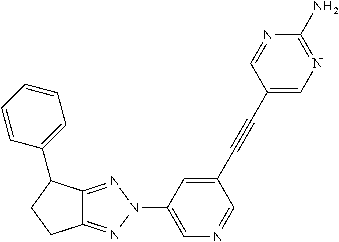 [SMILES: Nc1ncc(C#Cc2cncc(-n3nc4c(n3)C(c3ccccc3)CC4)c2)cn1] | Compound 54 |
| 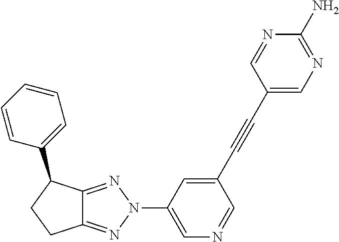 [SMILES: Nc1ncc(C#Cc2cncc(-n3nc4c(n3)[C@H](c3ccccc3)CC4)c2)cn1] | Compound 55 |
| 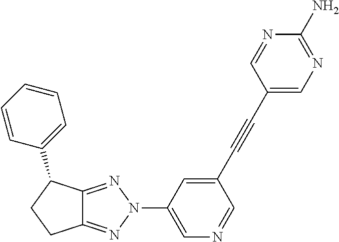 [SMILES: Nc1ncc(C#Cc2cncc(-n3nc4c(n3)[C@@H](c3ccccc3)CC4)c2)cn1] | Compound 56 |
| 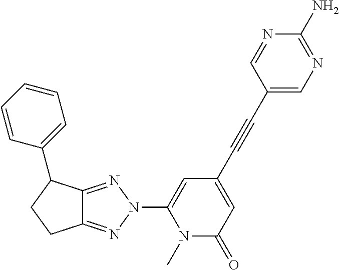 [SMILES: Cn1c(-n2nc3c(n2)C(c2ccccc2)CC3)cc(C#Cc2cnc(N)nc2)cc1=O] | Compound 57 |
| 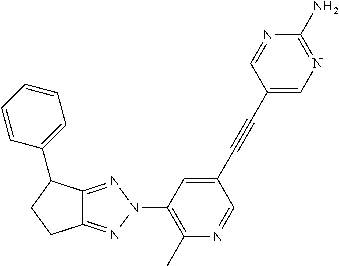 [SMILES: Cc1ncc(C#Cc2cnc(N)nc2)cc1-n1nc2c(n1)C(c1ccccc1)CC2] | Compound 58 |
| 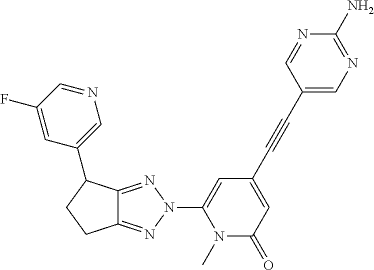 [SMILES: Cn1c(-n2nc3c(n2)C(c2cncc(F)c2)CC3)cc(C#Cc2cnc(N)nc2)cc1=O] | Compound 59 |
| 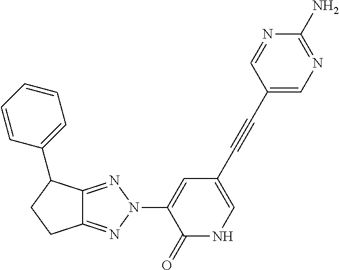 [SMILES: Nc1ncc(C#Cc2c[nH]c(=O)c(-n3nc4c(n3)C(c3ccccc3)CC4)c2)cn1] | Compound 60 |
| 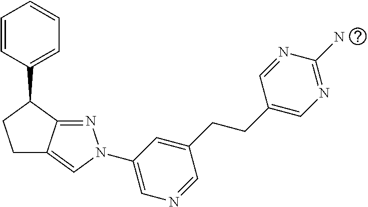 [SMILES: Nc1ncc(CCc2cncc(-n3cc4c(n3)[C@H](c3ccccc3)CC4)c2)cn1] | Compound 61 |
| 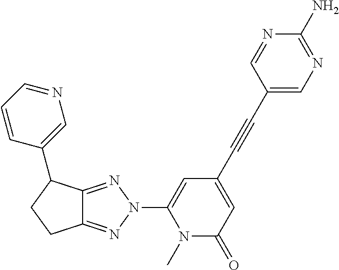 [SMILES: Cn1c(-n2nc3c(n2)C(c2cccnc2)CC3)cc(C#Cc2cnc(N)nc2)cc1=O] | Compound 62 |
| 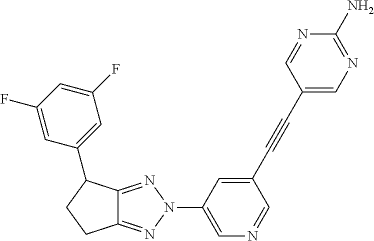 [SMILES: Nc1ncc(C#Cc2cncc(-n3nc4c(n3)C(c3cc(F)cc(F)c3)CC4)c2)cn1] | Compound 63 |
| 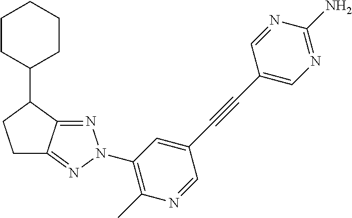 [SMILES: Cc1ncc(C#Cc2cnc(N)nc2)cc1-n1nc2c(n1)C(C1CCCCC1)CC2] | Compound 64 |
| 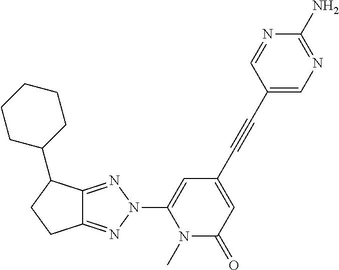 [SMILES: Cn1c(-n2nc3c(n2)C(C2CCCCC2)CC3)cc(C#Cc2cnc(N)nc2)cc1=O] | Compound 65 |
| 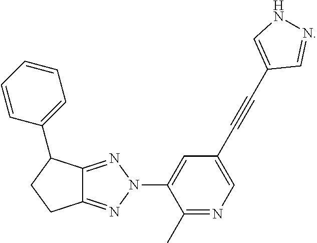 [SMILES: Cc1ncc(C#Cc2cn[nH]c2)cc1-n1nc2c(n1)C(c1ccccc1)CC2] | Compound 66 |

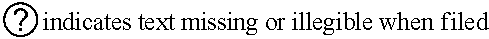

25. A pharmaceutical composition, which comprises a compound according to any one of claims 1-24, or a pharmaceutically acceptable salt, hydrate, solvate and stereoisomer thereof and a pharmaceutically acceptable excipient.

26. A compound according to any one of claims 1-24, or a pharmaceutically acceptable salt, hydrate, solvate and stereoisomer thereof, or a pharmaceutical composition comprising a compound according to any one of claims 1-24, or a pharmaceutically acceptable salt, hydrate, solvate and stereoisomer thereof and a pharmaceutically acceptable excipient, for use in treating or preventing a disease.

27. A compound according to any one of claims 1-24, or a pharmaceutically acceptable salt, hydrate, solvate and stereoisomer thereof, or a pharmaceutical composition comprising a compound according to any one of claims 1-24, or a pharmaceutically acceptable salt, hydrate, solvate and stereoisomer thereof and a pharmaceutically acceptable excipient, for use in inhibiting programmed necrosis, inhibiting RIP1 kinase, or treating or preventing a disease that is at least partially mediated by RIP1 kinase.

28. Use of a compound according to any one of claims 1-24, or a pharmaceutically acceptable salt, hydrate, solvate and stereoisomer thereof, or a pharmaceutical composition comprising a compound according to any one of claims 1-24, or a pharmaceutically acceptable salt, hydrate, solvate and stereoisomer thereof and a pharmaceutically acceptable excipient for inhibiting programmed necrosis, inhibiting RIP1 kinase, or treating or preventing a disease that is at least partially mediated by RIP1 kinase.

29. Use of a compound according to any one of claims 1-24, or a pharmaceutically acceptable salt, hydrate, solvate and stereoisomer thereof, or a pharmaceutical composition comprising a compound according to any one of claims 1-24, or a pharmaceutically acceptable salt, hydrate, solvate and stereoisomer thereof and a pharmaceutically acceptable excipient in manufacture of a medicament for inhibiting programmed necrosis, inhibiting RIP1 kinase, or treating or preventing a disease that is at least partially mediated by RIP1 kinase.

30. A method for inhibiting programmed necrosis, inhibiting RIP1 kinase, or treating or preventing a disease that is at least partially mediated by RIP1 kinase, wherein said method comprises administering to a subject in need thereof a therapeutically effective amount of a compound according to any one of claims 1-24, or a pharmaceutically acceptable salt, hydrate, solvate and stereoisomer thereof, or a pharmaceutical composition comprising a compound according to any one of claims 1-24, or a pharmaceutically acceptable salt, hydrate, solvate and stereoisomer thereof and a pharmaceutically acceptable excipient.
